(19) United States
(12) Patent Application Publication
(10) Pub. No.: US 2022/0127682 A1
(43) Pub. Date: Apr. 28, 2022

(54) SELECTION AND TREATMENT OF CANCER WITH COMBINATION THERAPIES

(71) Applicants: Massachusetts Institute of Technology, Cambridge, MA (US); Cardiff Oncology, Inc., San Diego, CA (US)

(72) Inventors: Michael B. Yaffe, Cambridge, MA (US); Jesse C. Patterson, Cambridge, MA (US); Mark Erlander, San Diego, CA (US); Peter J.P. Croucher, San Diego, CA (US); Maya Ridinger, San Diego, CA (US)

(21) Appl. No.: 17/459,477

(22) Filed: Aug. 27, 2021

Related U.S. Application Data
(60) Provisional application No. 63/077,157, filed on Sep. 11, 2020.
(60) Provisional application No. 63/093,657, filed on Oct. 19, 2020.

(51) Int. Cl. C12Q 1/6886; A61K 45/06

(52) U.S. Cl. CPC C12Q 1/6886; C12Q 2600/158; C12Q 2600/106; A61K 45/06

(57) ABSTRACT

Certain molecular characteristics of cancer cells or tumors can be indicative of their sensitivity to various combination therapies. The cancer cells or tumors exhibiting such molecular profiles, such as upregulation of genes associated with mitosis or meiosis, can be sensitive to additive and more than additive effects of combination therapies. Methods for identifying, selecting, and/or treating cancer patients whose cancer is amenable to combination therapies including an antiandrogen or androgen antagonist in combination with a Plk inhibitor are disclosed. Administration of the combination of the active agents can reduce cancer cell proliferation or viability and/or tumor burden in a subject with cancer to the same degree, or a greater degree than administering to the subject the same amount of either active agent alone.

### CROSS REFERENCE TO RELATED APPLICATIONS

This application claims the benefit of and priority to U.S. Provisional Application No. 63/077,157, filed Sep. 11, 2020, and U.S. Provisional Application No. 63/093,657, filed Oct. 19, 2020, which are hereby incorporated herein by reference in their entirety.

### STATEMENT REGARDING FEDERALLY SPONSORED RESEARCH

This invention was made with Government support under Grant No. R35 ES028374 awarded by the National Institutes of Health (NIH). The Government has certain rights in the invention.

### FIELD OF THE INVENTION

The invention is generally directed to combination therapies, and more particularly to combination therapies including an antiandrogen or androgen antagonist and a polo-like kinase inhibitor for the treatment of cancer in select patients.

### BACKGROUND OF THE INVENTION

Prostate cancer is the most frequently diagnosed non-skin related cancer and the second leading cause of cancer related deaths among men. A hallmark of prostate cancer is its dependence on androgen signaling through the androgen receptor (AR). While the efficacy of androgen-depletion therapy for the treatment of metastatic prostate cancer has been known for more than 70 years, patients frequently progress to androgen-independent or castrate-resistant prostate cancer (CRPC). The transition from androgen-dependent to castrate-resistant prostate cancer (CRPC) is a common characteristic of the progression of metastatic prostate cancer. Several second line antiandrogen therapies have been developed which further inhibit androgen signaling by competing with androgen for AR binding, disrupting testosterone synthesis, or both.

Abiraterone is an antiandrogen that is a standard-of-care for the treatment of prostate cancer. Abiraterone inhibits Cytochrome P450 17A1 (CYP17A1), thereby blocking the synthesis of testosterone and its derivatives. Some metabolites of abiraterone may directly antagonize the androgen receptor (AR). Most patients receiving abiraterone treatment ultimately become resistant. Resistance may be AR-dependent or AR-independent.

Treatment outcomes in cancers may benefit greatly from the use of therapies that combine multiple drugs. For example, drugs may have greater than additive effects either by targeting multiple oncogenic pathways that contribute to development of a cancer, or by independently reinforcing each other's effects on key processes driving the cancer. Such combination therapies are of particular interest due to their potential to increase efficacy and cancer cell selectivity, minimize the development of resistance, and allow for decreases in individual drug dosage required, possibly avoiding toxicity.

To this end, combination therapies for the treatment of CRPC are being explored. For example, combined treatment with PLK inhibitors and abiraterone has demonstrated greater than additive CRPC tumor cell killing (see e.g., U.S. Pat. Nos. 9,566,280 and 10,155,006). Clinical trials evaluating the safety and efficacy of onvansertib (an inhibitor of the PLK1, a master regulator of the cell cycle) in combination with abiraterone (Zytiga; abiraterone acetate) and prednisone in metastatic castrate resistant prostate cancer (mCRPC) are underway (ClinicalTrials.gov Identifier NCT03414034). However, such combinations may not be uniformly effective across all prostate cancer patients.

There exists an urgent need for improved combination therapies that effectively treat androgen-independent or castrate-resistant prostate cancer. There is also a need to identify the optimal drug combination for subgroups of patients with different molecular characteristics. There is a need to identify subgroups of patients whose tumors are likely to respond to specific combination therapies.

Therefore, it is an object of the invention to provide compositions and methods for identifying cancer patients whose cancers are sensitive to combination therapies.

It is also an object of the invention to provide compositions and methods for treating subpopulations of patients whose cancers are amenable to particular combination therapies.

### SUMMARY OF THE INVENTION

It has been discovered that certain molecular characteristics of cancer cells or tumors can be indicative of a favorable response of the cancer cells or tumors to treatment with one or more drugs. Methods for characterizing the gene expression and cytological profile of tumors or cancer cells from a subject having cancer are provided. These methods are useful in the diagnosis, prognosis, selection, and treatment of patients having cancer. The methods are useful in identifying cancer patients most likely to respond to combination therapy including antiandrogens or androgen antagonists and PLK inhibitors. Typically, the patients identified as likely to respond will show a greater reduction in cancer cell proliferation and/or viability, or tumor burden and/or progression, upon treatment with the combination therapy as compared to treatment with the antiandrogen or androgen antagonist alone or the PLK inhibitor alone. In certain embodiments, the cancers that are sensitive to additive and more than additive effects of the combination therapies are characterized by a specific gene or cytological profile. For example, cancer cells or tumors that express genes associated with proliferation (e.g., E2F target genes, genes involved in mitotic spindle assembly and/or checkpoint) can be more sensitive to the combination therapies than cancer cells that do not express these genes or express these genes to a lesser extent.

In particular, a method for selecting and treating a subject having cancer includes (a) analyzing the expression of one or more genes or gene products involved in mitosis, meiosis, the mitotic spindle, mitotic spindle assembly and/or checkpoint, microtubule organization, the centromere, the kinetochore, G2M checkpoint, or E2F target genes in a sample from the subject, (b) selecting the subject for treatment if the sample is characterized by expression or up-regulation of the one or more genes, and (c) administering to the selected subject an effective amount of an antiandrogen or androgen antagonist in combination with an effective amount of a Plk inhibitor.

The subject may or may not have been previously administered an antiandrogen or androgen antagonist. The subject-derived sample can include cancer cells, a blood sample, bone-marrow aspirate, ascites fluid, or a tumor biopsy. The analysis can be performed on isolated tumor cells, circulating tumor cells (CTCs) or cell-free RNA/exosomal RNA isolated from the blood sample. Typically, analyzing the expression of the one or more genes or gene products involves quantification of RNA and/or protein expression (e.g., via in situ hybridization, qPCR, RNA-sequencing, flow cytometry). The analysis can further include quantification of genomic amplification or copy number variation, a cytological assay, or combinations thereof.

In some embodiments, it is advantageous to treat the sample with the antiandrogen or androgen antagonist before performing the analysis of gene expression. In some embodiments, for example when the patient has not been previously treated with the antiandrogen or androgen antagonist, the sample (e.g., cancer cells, CTCs) is treated with an antiandrogen or androgen antagonist before analyzing gene expression. In some embodiments, for example patients who have previously received or are currently receiving the antiandrogen or androgen antagonist, the sample (e.g., cancer cells, CTCs) can be cultured independently of the antiandrogen or androgen antagonist for a period of time, followed by subsequent treatment with the antiandrogen or androgen antagonist.

The analysis allows one to determine if one or more genes are expressed or up-regulated following treatment with (e.g., as a result of) the antiandrogen or androgen antagonist. This can be relative to the expression of the one or more genes in a sample from a patient not treated with the antiandrogen or androgen antagonist or to the same sample prior to treatment of the patient with the antiandrogen or androgen antagonist. In some embodiments, the sample exhibits expression or up-regulation of the one or more genes relative to their expression in a sample from a subject who has not been administered the antiandrogen or androgen antagonist.

A method of treating cancer in a subject in need thereof, wherein the cancer is characterized by expression or up-regulation of one or more genes or gene products involved in mitosis, meiosis, the mitotic spindle, mitotic spindle assembly and/or checkpoint, microtubule organization, the centromere, the kinetochore, G2M checkpoint, E2F target genes, or combinations thereof has been developed. Typically, the method includes administering an effective amount of an antiandrogen or androgen antagonist in combination with an effective amount of a Plk inhibitor to the subject, then looking for an effect on tumor specific gene expression, especially those genes involved in meiosis or mitosis. In some embodiments, the subject has previously received or is currently receiving the antiandrogen or androgen antagonist prior to the administration of the combination therapy. In some embodiments, the expression or up-regulation of the one or more genes or gene products is relative to their expression in a cancer from a subject who has not been administered the antiandrogen or androgen antagonist.

In any of the methods, the one or more genes or gene products can be selected from Table 1 and/or Table 2.

Typically, administration of the combination of the two active agents (e.g., antiandrogen or androgen antagonist and Plk inhibitor) is effective to reduce cancer cell proliferation and/or viability in a subject with cancer. In some embodiments, the reduction is to a greater degree than administering to the subject the same amount of antiandrogen or androgen antagonist alone or the same amount of Plk inhibitor alone. In preferred embodiments, the reduction in cancer cell proliferation or viability in the subject with cancer is more than the additive reduction achieved by administering the antiandrogen or androgen antagonist alone or the Plk inhibitor alone. In some embodiments, in subjects with tumors, the combination is effective to reduce tumor burden, reduce tumor progression, or a combination thereof.

The antiandrogen or androgen antagonist and the Plk inhibitor can be administered on the same or different day. In some embodiments, the two agents are administered simultaneously. The antiandrogen or androgen antagonist and the Plk inhibitor can be administered as separate compositions. In some embodiments, the separate compositions are administered through the same route of administration. In other embodiments, the separate compositions are administered through different routes of administration. For example, in some embodiments, an antiandrogen or androgen antagonist is administered orally, and a PLK1 inhibitor is administered intravenously (e.g., via injection or infusion).

In some embodiments, the antiandrogen or androgen antagonist is administered prior to administration of the Plk inhibitor. For example, the antiandrogen or androgen antagonist can be administered 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the Plk inhibitor. In other embodiments, the PLK1 inhibitor is administered prior to administration of the antiandrogen or androgen antagonist. For example, the Plk inhibitor can be administered 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the antiandrogen or androgen antagonist.

In some embodiments, the antiandrogen or androgen antagonist and Plk inhibitor are administered in cycles. Dosing cycles could be of any length (e.g., 7, 14, 21, or 28 days), and the days in which the antiandrogen or androgen antagonist and the Plk inhibitor are administered can be completely or partially overlapping. For example, in some embodiments, the antiandrogen or androgen antagonist is administered daily and the Plk inhibitor is administered on days 1 through 5 of a 14 or 21 day cycle. In some embodiments, the antiandrogen or androgen antagonist is administered daily and the Plk inhibitor is administered on days 1 through 14 of a 21 day cycle. Different dosing schedules may utilize different dosages of the antiandrogen or androgen antagonist or Plk inhibitor.

Exemplary antiandrogens or androgen antagonists include TOK-001, and abiraterone or a prodrug, analog, derivative, or pharmaceutically acceptable salt thereof. An exemplary abiraterone prodrug is abiraterone acetate. The dosage of abiraterone acetate can be, for example, in the range of 250-1,500 mg, inclusive. Representative Plk inhibitors include dihydropteridinones, pyridopyrimidines, aminopyrimidines, substituted thiazolidinones, pteridine derivatives, dihydroimidazo[1,5-f]pteridines, metasubstituted thiazolidinones, benzyl styryl sulfone analogues, 4,5-dihydro-1H-pyrazolo[4,3-h]quinazoline derivatives, and stilbene derivatives. The Plk inhibitor is preferably a PLK1 inhibitor. Exemplary Plk inhibitors include onvansertib (NMS-1286937), BI2536, volasertib (BI 6727), GSK461364, HMN-176, HMN-214, rigosertib (ON-01910), MLN0905, TKM-080301, TAK-960, and Ro3280. Preferred PLK1 inhibitors include onvansertib, BI2536 and volasertib. The dosage of onvansertib can be in the range of 6 to 60 mg/m², inclusive. The dosage of BI2536 or volasertib can be in the range of 1-500 mg, inclusive. In particular embodiments, the dosage of volasertib is between about 1 and 300 mg, inclusive, or between 1 and 300 mg/m², inclusive.

The methods can include administering an additional therapy to the subject. For example, in some embodiments, the methods also include surgery and/or radiation therapy. Additionally, the subject can be treated with an anti-inflammatory such as a steroid (e.g., prednisone which is often administered in combination with abiraterone), a chemotherapeutic agent (e.g., docetaxel), an anti-infective agent, a hematopoietic agent (such as an agent that stimulates white blood cell production such as granulocyte colony-stimulating factor (G-CSF)) or combinations thereof.

These compositions and methods are particularly effective for treating prostate cancer, breast cancer, ovarian cancer, colorectal cancer, pancreatic cancer, head and neck cancer, bladder cancer, and acute myeloid leukemia. In some embodiments, the prostate cancer is an androgen-insensitive prostate cancer (e.g., castrate resistant prostate cancer). The subject (e.g., patient) is preferably a human.

### BRIEF DESCRIPTION OF THE DRAWINGS

FIGS. 1A-1B are graphs showing the percentage viability of C4-2 castrate resistant prostate cancer cells in the presence of increasing concentrations of abiraterone either in the absence (black line; [symbol] ) or presence (grey line; [symbol] ) of a constant amount of the PLK1 inhibitor BI2536. The predicted additive result of the two agents in combination is shown as “expected” (dashed black line; [symbol] ) and was calculated using the Bliss independence model of drug additivity (The toxicity of poisons applied jointly, C. I. Bliss, Annals of Applied Biology, 26(3): 585-615 (1939)). Relative viability was assessed after 72 hours using Cell Titre Glo™ according to the manufacturer's recommendations. More than additive effect (e.g., synergy) is a decrease in cell viability from the expected (dashed black line; [symbol] ) to observed (grey line; [symbol] ) dose response curves. These experiments were done both in the absence or presence of exogenous androgens by growing these cells in charcoal-stripped fetal bovine serum (FBS; FIG. 1A) or non-stripped FBS (FIG. 1B), respectively. All subsequent experiments were performed in FBS unless otherwise stated. FIGS. 1C-1E are graphs showing the percentage viability of C4-2 CRPC cells after treatment with increasing concentrations of abiraterone in the absence (black line; [symbol] ) or presence (grey line; [symbol] ) of the indicated amount of PLK1 inhibitors (onvansertib—FIG. 1C, volasertib—FIG. 1D; GSK461364—FIG. 1E). Cells were treated, relative viability assessed, and data plotted as in FIG. 1A. More than additive effect (e.g., synergy) is a decrease in cell viability from the expected (dashed black line; [symbol] ) to observed (grey line; [symbol] ) dose response curves. FIGS. 1F-1G are graphs showing the response (viability) of androgen-dependent prostate cancer cells (LNCaP) to increasing concentrations of abiraterone in the absence (black line; [symbol] ) or presence (grey line; [symbol] ) of the PLK1 inhibitor onvansertib (FIG. 1F) or BI2536 (FIG. 1G). The expected effect is shown in the dashed black line( [symbol] ). Cells were treated, relative viability assessed, and data plotted as in FIG. 1A. No greater than additive effect was seen in these androgen dependent prostate cancer cells, indicating this phenotype is acquired by C4-2 CRPC cells during their acquisition of a castrate-resistant state.

FIGS. 2A-2B show that abiraterone enhances the mitotic arrest caused by onvansertib treatment and exacerbates subsequent apoptotic cell death. FIG. 2A is a line graph showing the percentage of serine 10 phosphorylated histone H3 (pHH3) positive C4-2 CRPC cells as a measure of mitotic cells. FIG. 2B is a line graph showing the percentage of cleaved Caspase-3 (CC3) positive C4-2 CRPC cells as a measure of apoptotic cell death. Cells were subjected to the indicated concentrations of abiraterone and onvansertib for the indicated amount of time, collected, fixed and stained with antibodies that recognize pHH3 and CC3. The percentage of cells that stained positive with these antibodies was measured by flow cytometry using standard methods. In FIGS. 2A-2B, [symbol] represents DMSO; [symbol] represents abiraterone; [symbol] represents onvansertib; and [symbol] represents abiraterone and onvansertib.

FIG. 3 is a graph showing the percentage viability of C4-2 castrate resistant prostate cancer (CRPC) cells in the presence of increasing concentrations of abiraterone either in the absence (black line; [symbol] ) or presence (grey line; [symbol] ) of Docetaxel. The predicted additive result of the two agents in combination is shown as “expected” (dashed black line; [symbol] ). These data were acquired with C4-2 cells grown in charcoal-stripped FBS (similar results were obtained when cells were grown in non-stripped FBS). Cells were treated and data was analyzed as in FIGS. 1A-1G.

FIG. 4A shows structural formula for abiraterone, testosterone, and enzalutamide. Abiraterone is a steroid derivative that resembles naturally occurring androgens. The direct AR-antagonist, enzalutamide, does not resemble naturally occurring androgens. FIGS. 4B-4C are graphs showing the percentage viability of C4-2 CRPC cells in the presence of increasing concentrations of enzalutamide either in the absence (black line; [symbol] ) or presence (grey line; [symbol] ) of the PLK1 inhibitor onvansertib (FIG. 4B) or BI2536 (FIG. 4C). The predicted additive result of the two agents in combination is shown as “expected” (dashed black line; [symbol] ). Cells were treated and data was analyzed as in FIGS. 1A-1G. FIG. 4D shows that enzalutamide does not enhance the mitotic arrest causes by the PLK1 inhibitor onvansertib. C4-2 CRPC cells were treated with the indicated drugs for 16 hours, fixed and stained with antibodies that detect pH3 as a measure of mitotic cells as in FIGS. 2A-2B.

FIGS. 5A-5C are graphs showing the percentage viability of acute myeloid leukemia (MV-4-11; FIG. 5A), pancreatic (PSN-1; FIG. 5B), and ovarian (SK-OV-3; FIG. 5C) cancer cells in the presence of increasing concentrations of abiraterone either in the absence (black line; [symbol] ) or presence (grey line; [symbol] ) of a constant amount of the PLK1 inhibitor. The dose of onvansertib was 22.2 nM, 22.2 nM and 33.3 nM for MV-4-11, PSN-1 and SK-OV-3, respectively. The predicted additive result of the two agents in combination is shown as “expected” (dashed black line; [symbol] ). The cells were treated and data analyzed as in FIGS. 1A-1G. These non-prostate cancer cells have a greater than additive response to the combination of abiraterone and PLK1 inhibitor. FIGS. 5D-5F are graphs showing the percentage viability of acute myeloid leukemia (OCI-AML-3; FIG. 5D), pancreatic (Panc 10.05; FIG. 5E), and ovarian (OAW28; FIG. 5F) cancer cells in the presence of increasing concentrations of abiraterone either in the absence (black line; [symbol] ) or presence (grey line; [symbol] ) of a constant amount of the PLK1 inhibitor. The dose of onvansertib was 22.2. nM, 33.3 nM and 15 nM for OCI-AML-3, Panc 10.05 and OAW28, respectively. The predicted additive result of the two agents in combination is shown as “expected” (dashed black line; [symbol] ). The cells were treated and data analyzed as in FIGS. 1A-1G. These non-prostate cancer cells do not have a greater than additive response to the combination of abiraterone and PLK1 inhibitor. Unlike C4-2 CRPC cells, these non-prostate cancer cell lines lack detectable AR protein.

FIG. 6 is a schematic representation of the RNA-Seq analysis pipeline.

FIGS. 7A and 7B are schematic representations of the set-based analyses performed on data from synergistic (FIG. 7A) and non-synergistic LNCaP (FIG. 7B) cells. Four groups of gene sets were gathered each from the datasets and include: Group I—Differentially enriched gene sets common to treatment with abiraterone and onvansertib in responder cell lines (A) ABI_{Syn}∪ONV_{Syn}; Group II—Differentially enriched gene sets common to treatment with abiraterone and onvansertib in non-responder cell lines (B) ABI_{NonSyn}∪ONV_{NonSyn}; Group III—Differentially enriched gene sets unique to the abiraterone plus onvansertib combination in responder cell lines (C) and not differentially enriched in non-responder cell line contrast (ABI+ONV_{Syn}/ABI_{Syn}∪ONV_{Syn})/(ABI+ONV_{NonSyn}∪(ABI_{NonSyn}∪ONV_{NonSyn}); and Group IV—Differentially enriched gene sets unique to the abiraterone plus onvansertib combination in non-responder cell lines (D) and not differentially enriched in responder cell line contrast (ABI+ONV_{NonSyn}/ABI_{NonSyn}∪ONV_{NonSyn})/(ABI+ONV_{Syn}∪(ABI_{Syn}∪ONV_{Syn}).

FIGS. 8A-8D are schematic representations of the set-based analyses of the responder C4-2 cell line (FIG. 8A), the non-responder LNCaP cell line (FIG. 8B), other non-prostate cancer responder cell lines (FIG. 8C), and other non-prostate cancer non-responder cell lines (FIG. 8D).

FIGS. 9A and 9B are graphs showing in vivo greater than additive effect of the PLK1i, BI2536 and Abiraterone, on induction of apoptotic cancer cell death. A tumor-implantable device containing multiple microwells generating drug gradients in C4-2 tumor-bearing mice (FIG. 9A) and SK-OV-3 tumor-bearing mice (FIG. 9B) was used. Fixed tumor sections adjacent to the microwells diffusing vehicle, Abi, PLK1i or Abi+PLK1i were stained with antibodies which bind cleaved-caspase 3 to detect apoptotic cells.

FIG. 10 is a graph showing change in Abi-Onv synergy signature score in prostate cancer specimens characterized into four molecular subtypes: Luminal A (LA), Luminal Proliferating (LP), Basal Immune (BI) and Basal (B).

FIG. 11 is a flow chart showing steps in tumor transcriptomic profiling to predict clinical response.

FIG. 12 is a schematic of the androgen receptor signaling pathway.

FIG. 13 is a schematic of the sites at which enzalutamide and abiraterone interact with the androgen receptor and pathways associated with testosterone synthesis.

FIG. 14 is a schematic depiction of how spindle rotation was measured during progression through mitosis. C4-2 CRPC cells were engineered to express red fluorescent protein fused to the H2B histone and green fluorescent protein fused to tubulin so that mitotic spindles in dividing cells could be tracked over time by live-cell time-lapse microscopy. The orientation of the spindle's major axis was tracked over time in individual cells. Both the cumulative angular distance (total rotation during mitosis) and the net angular displacement (the difference between initial to final angle) were measured.

FIGS. 15A-15B are violin plots showing the distribution of net angular displacement (FIG. 15A) and cumulative angular distance (FIG. 15B) measurements from populations of cells treated with DMSO vehicle (white), abiraterone (right slant lines) or enzalutamide (left slant lines) (n≥92). The solid black line represents the median and dotted black lines are upper and lower quartiles. **p≤0.01, **** p≤0.0001, n.s. not significant using a two-tailed Mann-Whitney U test.

FIG. 16 shows the percentage of in-plane or out-of-plane divisions upon treatment of cells with DMSO, abiraterone, or enzalutamide observed in C4-2 CPRC cells tracked by live-cell microscopy described in FIG. 14. Altered angles of cell division are consistent with the spindle orientation defects observed in these cells upon treatment with abiraterone.

FIG. 17 is a schematic depiction of the roles of PLK1 in cell replication, including spindle assembly.

### DETAILED DESCRIPTION OF THE INVENTION

### I. Definitions

As used herein, the term “combination therapy” refers to treatment of a disease or symptom thereof, or a method for achieving a desired physiological change, including administering to an animal, such as a mammal, especially a human being, an effective amount of two or more chemical agents or components to treat the disease or symptom thereof, or to produce the physiological change, wherein the chemical agents or components are administered together, such as part of the same composition, or administered separately and independently at the same time or at different times (e.g., administration of each agent or component is separated by a finite period of time from each other).

As used herein, the term “dosage regime” refers to drug administration regarding formulation, route of administration, drug dose, dosing interval and treatment duration.

As used herein, the terms “individual”, “host”, and “subject” are used interchangeably, and refer to a mammal, including, but not limited to, primates, for example, human beings, as well as rodents, such as mice and rats, and other laboratory animals. A “patient” refers to a subject afflicted with a disease or disorder and includes human and veterinary subjects.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered. The effect of the effective amount can be relative to a control. Such controls are known in the art and are discussed throughout. Suitable controls include, the condition of the subject or sample from the subject prior to or in the absence of administration of the drug, or drug combination, or in the case of drug combinations, the effect of the combination can be compared to the effect of administration of only one of the drugs. In some embodiments, a control can be the condition of a reference subject or sample from a reference subject who has not been administration the drug or combination of drugs.

The term “tumor cell” or “cancer cell”, denotes a cell which may be malignant (e.g., capable of metastasis and the mediation of disease), or benign. In contrast, a “non-tumor cell” is a normal cell (which may be quiescent or activated) that can be located within a tumor microenvironment, including, but not limited to, Tumor Infiltrating Lymphocytes (TILs), leucocytes, macrophages, and/or other cells of the immune system, and/or stromal cells, and/or fibroblasts (e.g., cancer or tumor associated fibroblasts). The tumor cells include, but are not limited to, tumor cells of any cancer, such as leukemias (e.g., acute leukemia, acute lymphocytic leukemia, acute myelocytic leukemias, chronic leukemias such as chronic myelocytic (granulocytic) leukemia, chronic lymphocytic leukemia), lymphomas, multiple myelomas, bone and connective tissue sarcomas (e.g., bone sarcoma, osteosarcoma, chondrosarcoma, fibrosarcoma), brain tumors, breast cancer, thyroid cancer, pituitary cancer, vaginal cancers, vulvar cancer, cervical cancers, uterine cancers, ovarian cancers, esophageal cancers, stomach cancers, colon cancers, rectal cancers, liver cancers, lung cancers, testicular cancers, prostate cancers, penal cancers, oral cancers, basal cancers, salivary gland cancers, pharynx cancers, skin cancers, kidney cancers, and bladder cancers.

The term “cell(s) of a tumor” is employed to refer to tumor cells and non-tumor cells located within a tumor or a tumor environment. The subject (e.g., patient) and the tumors to be characterized may be of any mammalian species (e.g., human, or primate, canine, feline, bovine, ovine, equine, porcine, rodent species (e.g., murine), etc.). The disclosure particularly concerns the characterization of human tumor cells as well as the characterization of human tumor microenvironments.

The term “characterizing” is intended to refer to assessing a patient, tissue sample, cell free sample, or cell(s) for the expression or presence of a biomarker. A biomarker refers generally to a molecule, including a gene or product thereof, nucleic acids (e.g., DNA, RNA), protein/peptide/polypeptide, carbohydrate structure, lipid, glycolipid, characteristics of which can be detected in a tissue or cell to provide information that is predictive, diagnostic, and/or prognostic (e.g., for sensitivity or resistance to candidate treatment). In some embodiments, the biomarker can include, a gene or gene product (e.g., which may be expressed on the surface of or within a cell or tissue), chromosomal aberrations, genomic amplifications or copy number variations, and physical cell cycle related structures or compartments (e.g., spindle assembly, centriole location/number, chromosome alignment, chromatin structure). In some embodiments, characterization is mediated using molecules that physiospecifically or immunospecifically bind to the aforementioned biomarkers (e.g., gene product).

The term “inhibit” or “reduce” and other forms of the words such as “inhibiting” or “reducing” means to decrease, hinder or restrain a particular characteristic such as an activity, response, condition, disease, or other biological parameter. It is understood that this is typically in relation to some standard or expected value, but that it is not always necessary for the standard or relative value to be referred to. “Inhibits” or “reduce” can also mean to hinder or restrain the synthesis, expression or function of a protein relative to a standard or control. “Inhibits” or “reduce” can also mean to hinder or restrain cancer cell proliferation, viability, tumor burden, or combinations thereof. Inhibition/reduction can include, but is not limited to, the complete ablation of the activity, response, condition, or disease. For example, the term encompasses a 10% reduction in the activity, response, condition, disease, or other biological parameter as compared to the native or control level. In some embodiments, the reduction can be about 1 to 100%, or any integer there between, or any amount of reduction in between as compared to native or control levels.

“Treatment” or “treating” means to administer a therapy to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

“Tumor burden” refers to the number of cancer cells, the size or mass of a tumor, or the total amount of tumor/cancer in a particular region of a subject. Methods of determining tumor burden for different contexts are known in the art, and the appropriate method can be selected by the skilled person. For example, in some forms tumor burden may be assessed using guidelines provided in the Response Evaluation Criteria in Solid Tumors (RECIST).

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio. The term “pharmaceutically acceptable salt”, refers to derivatives of the compounds, wherein the parent compound is modified by making acid or base salts thereof. Examples of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

“Prodrug”, as used herein, refers to a pharmacological substance (e.g., drug) which is administered in an inactive (or significantly less active) form. Once administered, the prodrug is metabolized in the body (in vivo) into the active compound.

### II. Methods of Selection and Treatment

Cancer cells that exhibit a greater than additive effect (e.g., cell death/viability) from treatment with the combination of onvansertib and abiraterone exhibit a characteristic gene expression profile. As shown in the Examples, RNA-sequencing identified genes that represent biological pathways and processes that are associated with the greater than additive effect (e.g., synergy) between onvansertib and abiraterone in cancer cells. These genes (referred to as gene signature) are differentially expressed by cells sensitive to the greater than additive effect of the combination therapy (these cells are also referred to as responders), but not by cells which do not show a greater than additive effect (non-responders) to the combination therapy (e.g., onvansertib and abiraterone). This observation held true across many cancer cell types, including cells that do not express the androgen receptor (AR) at detectable levels.

The Examples show that the greater than additive response of cancer cells to the combination of abiraterone and onvansertib is primarily a result of differential response by the cells to abiraterone, and not to onvansertib. The differential response to abiraterone affects aspects of pathways and processes that onvansertib affects. As such, treatment of cancer cells with an antiandrogen or androgen antagonist appears to induce increased expression of specific genes that can enhance the sensitivity of cancer cells to Plk inhibition. Thus, it is believed that the response to abiraterone primes or sensitizes the response to onvansertib in certain cells.

The gene signature involves the abiraterone-dependent upregulation of sets of genes involved in cell division/proliferation, such as, genes involved in mitosis and meiosis (e.g., particularly related to spindle formation, chromosome alignment and attachment to the spindle, spindle checkpoint, microtubule events associated with cytokinesis, mitotic-G2/M checkpoint, and E2F target genes). These genes differ from those canonically thought to be directly regulated by the androgen receptor. Thus, the gene signature includes genes or subgroups of genes that are distinct from genes that are directly downstream from the androgen receptor (AR) pathway through which abiraterone is classically thought to act.

Expression or upregulation of one or more genes from the gene signature can function as an indicator or biomarker to identify patient tumors that will respond favorably to the combination treatments of an antiandrogen or androgen antagonist in combination with a Plk inhibitor (e.g., abiraterone and a PLK1 inhibitor). The gene signature and component genes thereof are useful in methods for diagnosis, prognosis, identification or selection, and/or treatment of patients having cancer.

This is the basis of the methods for identifying a cancer patient whose cancer cells or tumor associated cells are sensitive to treatment with antiandrogens or androgen antagonists in combination with Plk inhibitors. The methods involve analyzing or characterizing the gene expression profile of a sample from the subject cells (e.g., cancer cells, tumor tissue) to determine whether they exhibit expression or upregulation of one or more genes or gene sets which indicate that the cancer or tumor will be sensitive to the combination treatments of an antiandrogen or androgen antagonist. The methods can alternatively or additionally include performing a cytological assessment on cells to evaluate for example, spindle assembly, centriole location/number, chromosome alignment, and/or chromatin structure, which when aberrant can indicate that the cancer or tumor will be sensitive to the combination treatments of an antiandrogen or androgen antagonist and Plk inhibitor.

Methods for selecting subjects (e.g., cancer patients) for treatment with the therapies are also provided. Typically, the methods of selection also include identifying a subject whose cancer cells or tumor associated cells are sensitive to treatment with the combination therapies (e.g., through gene expression analysis of a subject-derived sample). For example, patients having cancer cells that express or upregulate genes involved in cell division/proliferation such as genes involved in mitosis and meiosis (in particular, genes from the signature discussed in more detail below) can be selected for treatment with the therapies.

Also provided are methods for treating a subject with any of the combination therapies. In some embodiments, the methods of treatment involve the identification and/or selection of specific subjects for treatment. For example, in some embodiments, the method of treatment includes methods for identifying and/or selecting patients who would be amenable for antiandrogen/androgen antagonist and the Plk inhibitor combination therapies, and for treating such patients.

Methods of treating cancer or one or more symptoms of cancer in a subject are provided. In certain embodiments, the methods include administering to a subject with cancer an effective amount of an androgen antagonist or antiandrogen, or a derivative, analog or prodrug, or a pharmacologically active salt thereof in combination with one or more inhibitors of Plk to reduce or inhibit one or more symptoms of the cancer. In preferred embodiments, the androgen antagonist or antiandrogen and inhibitors of Plk can be used in combination to provide enhanced antitumor activity as compared to the use of either agent alone. The methods can include contacting one or more cancer cells with an effective amount of an androgen antagonist or antiandrogen in combination with one or more inhibitors of Plk to decrease or inhibit the proliferation and/or viability of the cancer cells (e.g., compared to untreated control cancer cells).

An exemplary method includes selecting and treating a subject having cancer by (a) analyzing the expression of one or more genes involved in mitosis, meiosis, the mitotic spindle, mitotic spindle assembly and/or checkpoint, microtubule organization, the centromere, the kinetochore, G2M checkpoint, E2F target genes in a sample from the subject, (b) selecting the subject for treatment if the sample is characterized by expression or up-regulation of the one or more genes, and (c) administering to the selected subject an effective amount of an antiandrogen or androgen antagonist in combination with an effective amount of a Plk inhibitor. Another exemplary method involves treating cancer in a subject in need thereof by administering an effective amount of the combination therapy, wherein the cancer is characterized by expression or up-regulation of one or more genes or gene products associated with sensitivity to more than additive effects of an androgen antagonist or antiandrogen in combination with one or more inhibitors of Plk (e.g., genes involved in cell proliferation/division such as, mitosis or meiosis, such as genes listed in Tables 1 and 2).

In some embodiments, the subject may have been previously administered one or more of the drugs, but not in combination. It will be appreciated that the cancer may have developed a resistance to the previously administered active agent, when the agent is administered in the absence of the combination. Therefore, in some embodiments, the subject population being treated is defined as one in which the cancer being treated is resistant or insensitive to one or the other of the active agent when administered alone.

In certain embodiments, the methods are more effective in treating cancer in a subject having cancer cells that exhibit expression or upregulation of one or more genes involved in cell division/proliferation, such as, genes involved in mitosis and meiosis, such as genes listed in Tables 1 and 2. The genes involved in cell division/proliferation can include, genes related to spindle formation, chromosome alignment to the spindle, spindle checkpoint, microtubule events associated with cytokinesis, mitotic-G2/M checkpoint, and E2F target genes. In some embodiments, the cancer cells exhibit expression or upregulation of these genes relative to cells that have not been exposed to an antiandrogen or androgen antagonist. For example, in some embodiments, the methods are more effective in treating cancer in a subject having cancer cells that exhibit expression or upregulation of one or more genes that contain binding sites for an E2F transcription factor adjacent to their transcription start sites.

The antiandrogen or androgen antagonist and Plk inhibitor can be administered locally or systemically to the subject, or coated or incorporated onto, or into a device.

In a specific embodiment, the method is for identifying cancer patients most likely to respond to the combination therapies. In a particular embodiment, circulating tumor cells (CTCs) are isolated from a blood sample from the patient and cultured ex vivo. Cultured CTCs from abiraterone naïve patients can then be appropriately challenged with an antiandrogen or androgen antagonist (e.g., abiraterone). Alternatively, patients who have previously or who are currently receiving an antiandrogen or androgen antagonist (e.g., abiraterone) may have their CTCs cultured independently of the antiandrogen or androgen antagonist, with subsequent further antiandrogen or androgen antagonist (e.g., abiraterone) challenge. The effects of the drug challenge can be assessed on various aspects of cell proliferation/division such as mitotic spindle assembly and/or function, or gene expression. This can be accomplished by various means, including: (i) whole transcriptome RNASeq of abiraterone challenged cultured CTCs (with comparison to non-challenged cultured CTCs if available); (ii) targeted RNA sequencing or other quantitative approaches (e.g. qPCR, ddPCR, single-cell RNASeq, microarrays, NANOSTRING®, LUMINEX® assays) for quantifying expression of specific genes central to the modulated pathways/processes; (iii) measurement may also be made of other indirect genomic or genetic effectors of mitotic spindle assembly and/or function (e.g., somatic mutations, genomic amplifications, copy number variation, microRNA quantification (miRNASeq), upstream transcriptions factors); (iv) protein based measurements on CTCs such as flow cytometry, immunofluorescence microscopy, mass spectrometry or other techniques that can be used to measure changes in abundance or modification of mitotic proteins and spindle components; and (v) combinations thereof. The assay used to evaluate the effects of the drug challenge can be phenotypic, biochemical, or immunohistochemical based upon the presentation of physical cell cycle related structure or compartments (e.g., spindle assembly, centriole location/number, chromosome alignment, chromatin structure).

While there are several advantages of treating CTCs ex vivo, other methods of assessing the effect the antiandrogen or androgen antagonist (e.g., abiraterone) has on mitotic processes in a patient's cancer cells can be utilized. For example, patient biopsies can be collected before and after administration of the antiandrogen or androgen antagonist; patient biopsies can be dissociated, cultured ex vivo in 2D or 3D cell culture and the effects of the antiandrogen or androgen antagonist measured; cell-free and/or exosomal RNA can be analyzed in a patient before and after the antiandrogen or androgen antagonist treatment; circulating tumor DNA (ctDNA) can be analyzed before and after the antiandrogen or androgen antagonist treatment; or patient biopsies can be implanted in mice and treated with the antiandrogen or androgen antagonist in an animal setting. In some embodiments, patients currently on antiandrogen or androgen antagonist (e.g., abiraterone) treatment can be stratified by expression levels, in their cancer cells, of the genes and gene sets associated with sensitivity to more than additive effects of the combination therapies, wherein higher expression is associated with greater likelihood to respond to the combination therapy. In some embodiments, patients currently on antiandrogen or androgen antagonist (e.g., abiraterone) treatment with greater likelihood to respond to the combination therapies can be identified on the basis of elevated expression of the genes and gene sets associated with sensitivity to more than additive effects of the combination therapies, relative to patients that are not currently on antiandrogen or androgen antagonist (e.g., abiraterone) treatment.

A. Methods for Identifying and Selecting Patients

Methods for identifying and/or selecting specific subjects (e.g., for treatment with the compositions) have been developed. Typically, the subject has a tumor, tumor associated cells or cancer cells that are sensitive to treatment with antiandrogens or androgen antagonists in combination with Plk inhibitors. The methods for identifying and/or selecting can include characterizing subject-derived samples. For example, the methods can include determining whether cancer cells from a subject express or upregulate one or more genes associated with sensitivity to more than additive effects of an androgen antagonist or antiandrogen in combination with one or more inhibitors of Plk (e.g., genes involved in cell proliferation/division such as, mitosis or meiosis). In some embodiments, the expression or upregulation can be in response to treatment with an antiandrogen or androgen antagonist or relative to sample that has not been treated with an antiandrogen or androgen antagonist.

Methods for assessing the amenability of subject to a proposed anti-cancer therapy are also provided. The methods can include, for example, characterizing cells of a tumor of the subject by determining whether the cells of the tumor express or upregulate genes that are associated with sensitivity to more than additive effects of an androgen antagonist or antiandrogen in combination with one or more inhibitors of Plk (e.g., genes involved in cell proliferation/division such as, mitosis or meiosis).

Methods for selecting patients for anti-cancer therapy based on characterization of a subject-derived sample are also provided. In some embodiments, cancer patient samples are characterized prior to and following treatment with specific chemotherapeutic and/or biologic therapies, and/or other therapeutic interventions (e.g. radiation, treatment with an antiandrogen or androgen antagonist, etc.) in order determine whether the expression patterns of genes that are associated with sensitivity to more than additive effects of androgen antagonist or antiandrogen in combination with one or more inhibitors of Plk have changed.

1. Biomarkers

Genes and sets of genes that are associated with sensitivity to more than additive effects of the combination therapies have been identified. It is to be understood that reference to genes encompasses reference to the gene and/or gene product (e.g., mRNA, protein). For example, analyzing the expression of a gene includes evaluation of the gene or gene product (e.g., at the DNA, RNA, and/or protein levels). Genes can be coding or non-coding. For example, in some embodiments, microRNAs (miRNA), long noncoding RNAs (lncRNA), piwi-interacting RNAs (piRNAs), transfer RNAs (tRNAs), snoRNAs and ribosomal RNAs (rRNAs) can be analyzed.

Non-limiting examples of suitable genes include genes involved in or associated with cell division or proliferation (e.g., mitosis and meiosis). The genes involved in cell division or proliferation can include, genes related to spindle formation and/or function, chromosome alignment to the spindle, kinetochore formation, spindle checkpoint, microtubule organization, microtubule events associated with cytokinesis, chromosome segregation, centriole replication, centrosome duplication, mitotic-G2/M checkpoint, E2F target genes, and combinations thereof.

Exemplary genes and gene sets that are associated with sensitivity to more than additive effects of the combination of an androgen antagonist or antiandrogen and one or more Plk inhibitors are provided in Table 1 and Table 2.

TABLE 1
Genes (677) constituting the discrete cluster of 51 gene sets
selected by mean abiraterone and onvansertib significance
score and associated with response to the combination.

| RANK | GENE | NO. GENE SETS |
|---|---|---|
| 1 | CDC20 | 25 (49%) |
| 2 | NDC80 | 25 (49%) |
| 3 | CENPE | 24 (47%) |
| 4 | BIRC5 | 23 (45%) |
| 5 | CDCA8 | 22 (43%) |
| 6 | ZWINT | 22 (43%) |
| 7 | CCNB2 | 21 (41%) |
| 8 | CENPF | 21 (41%) |
| 9 | AURKA | 20 (39%) |
| 10 | AURKB | 20 (39%) |
| 11 | ASPM | 19 (37%) |
| 12 | CCNA2 | 19 (37%) |
| 13 | CDK1 | 19 (37%) |
| 14 | PLK1 | 19 (37%) |
| 15 | FOXM1 | 18 (35%) |
| 16 | HMMR | 18 (35%) |
| 17 | BUB1 | 17 (33%) |
| 18 | BUB1B | 17 (33%) |
| 19 | KIF11 | 17 (33%) |
| 20 | NUSAP1 | 17 (33%) |
| 21 | PCNA | 17 (33%) |
| 22 | SMC2 | 17 (33%) |
| 23 | SMC4 | 17 (33%) |
| 24 | TTK | 17 (33%) |
| 25 | RRM1 | 16 (31%) |
| 26 | ESPL1 | 15 (29%) |
| 27 | KIF18B | 15 (29%) |
| 28 | RRM2 | 15 (29%) |
| 29 | TOP2A | 15 (29%) |
| 30 | PRC1 | 14 (27%) |
| 31 | TYMS | 14 (27%) |
| 32 | DLGAP5 | 13 (25%) |
| 33 | KIF2C | 13 (25%) |
| 34 | MCM2 | 13 (25%) |
| 35 | MELK | 13 (25%) |
| 36 | RACGAP1 | 13 (25%) |
| 37 | TPX2 | 13 (25%) |
| 38 | UBE2C | 13 (25%) |
| 39 | CKS2 | 12 (24%) |
| 40 | MCM4 | 12 (24%) |
| 41 | PLK4 | 12 (24%) |
| 42 | CCNB1 | 11 (22%) |
| 43 | CENPA | 11 (22%) |
| 44 | GMNN | 11 (22%) |
| 45 | KIF20A | 11 (22%) |
| 46 | MKI67 | 10 (20%) |
| 47 | SHCBP1 | 10 (20%) |
| 48 | CENPM | 9 (18%) |
| 49 | FEN1 | 9 (18%) |
| 50 | H2AFX | 9 (18%) |
| 51 | MT1JP | 9 (18%) |
| 52 | NCAPD2 | 9 (18%) |
| 53 | NUF2 | 9 (18%) |
| 54 | RFC4 | 9 (18%) |
| 55 | CDCA3 | 8 (16%) |
| 56 | CLASP1 | 8 (16%) |
| 57 | FANCI | 8 (16%) |
| 58 | GINS2 | 8 (16%) |
| 59 | KIF4A | 8 (16%) |
| 60 | MAD2L1 | 8 (16%) |
| 61 | NCAPG | 8 (16%) |
| 62 | PAFAH1B1 | 8 (16%) |
| 63 | PPP2R1A | 8 (16%) |
| 64 | SGO1 | 8 (16%) |
| 65 | SPAG5 | 8 (16%) |
| 66 | BUB3 | 7 (14%) |
| 67 | CDCA5 | 7 (14%) |
| 68 | CENPC | 7 (14%) |
| 69 | CKAP5 | 7 (14%) |
| 70 | CLASP2 | 7 (14%) |
| 71 | DYNC1H1 | 7 (14%) |
| 72 | HMGB2 | 7 (14%) |
| 73 | INCENP | 7 (14%) |
| 74 | MAD1L1 | 7 (14%) |
| 75 | MCM3 | 7 (14%) |
| 76 | MIS12 | 7 (14%) |
| 77 | MYBL2 | 7 (14%) |
| 78 | NCAPH | 7 (14%) |
| 79 | NDE1 | 7 (14%) |
| 80 | NDEL1 | 7 (14%) |
| 81 | NEK2 | 7 (14%) |
| 82 | NSD2 | 7 (14%) |
| 83 | PTTG1 | 7 (14%) |
| 84 | RCC2 | 7 (14%) |
| 85 | RFC3 | 7 (14%) |
| 86 | SEH1L | 7 (14%) |
| 87 | SMC1A | 7 (14%) |
| 88 | SPC25 | 7 (14%) |
| 89 | SPDL1 | 7 (14%) |
| 90 | UBE2S | 7 (14%) |
| 91 | ZW10 | 7 (14%) |
| 92 | AURKC | 6 (12%) |
| 93 | CENPU | 6 (12%) |
| 94 | CKS1B | 6 (12%) |
| 95 | DSN1 | 6 (12%) |
| 96 | DYNC1LI1 | 6 (12%) |
| 97 | DYNLL1 | 6 (12%) |
| 98 | HJURP | 6 (12%) |
| 99 | HNRNPU | 6 (12%) |
| 100 | KIF18A | 6 (12%) |
| 101 | KIF2A | 6 (12%) |
| 102 | KNL1 | 6 (12%) |
| 103 | MCM6 | 6 (12%) |
| 104 | NSL1 | 6 (12%) |
| 105 | PPP1CC | 6 (12%) |
| 106 | PPP2CA | 6 (12%) |
| 107 | PPP2CB | 6 (12%) |
| 108 | PPP2R5D | 6 (12%) |
| 109 | PRIM1 | 6 (12%) |
| 110 | TMPO | 6 (12%) |
| 111 | XPO1 | 6 (12%) |
| 112 | AHCTF1 | 5 (10%) |
| 113 | ASF1B | 5 (10%) |
| 114 | CENPH | 5 (10%) |
| 115 | CENPI | 5 (10%) |
| 116 | CENPK | 5 (10%) |
| 117 | CENPL | 5 (10%) |
| 118 | CENPN | 5 (10%) |
| 119 | CENPO | 5 (10%) |
| 120 | CENPP | 5 (10%) |
| 121 | CENPQ | 5 (10%) |
| 122 | CENPS | 5 (10%) |
| 123 | CENPT | 5 (10%) |
| 124 | CLIP1 | 5 (10%) |
| 125 | DCTN1 | 5 (10%) |
| 126 | DCTN2 | 5 (10%) |
| 127 | DYNC1I1 | 5 (10%) |
| 128 | DYNC1I2 | 5 (10%) |
| 129 | DYNC1LI2 | 5 (10%) |
| 130 | ERCC6L | 5 (10%) |
| 131 | ITGB3BP | 5 (10%) |
| 132 | KIF2B | 5 (10%) |
| 133 | KIFC1 | 5 (10%) |
| 134 | KNTC1 | 5 (10%) |
| 135 | MAPRE1 | 5 (10%) |
| 136 | NUP107 | 5 (10%) |
| 137 | NUP133 | 5 (10%) |
| 138 | NUP160 | 5 (10%) |
| 139 | NUP37 | 5 (10%) |
| 140 | NUP43 | 5 (10%) |
| 141 | NUP62 | 5 (10%) |
| 142 | NUP85 | 5 (10%) |
| 143 | NUP98 | 5 (10%) |
| 144 | PCLAF | 5 (10%) |
| 145 | PMF1 | 5 (10%) |
| 146 | PPP2R1B | 5 (10%) |
| 147 | PPP2R5A | 5 (10%) |
| 148 | PPP2R5C | 5 (10%) |
| 149 | RAN | 5 (10%) |
| 150 | RANGAP1 | 5 (10%) |
| RANK | GENE | No. of. GeneSets |
| 151 | SEC13 | 5 (10%) |
| 152 | SGO2 | 5 (10%) |
| 153 | SKA1 | 5 (10%) |
| 154 | SKA2 | 5 (10%) |
| 155 | SMC3 | 5 (10%) |
| 156 | SPC24 | 5 (10%) |
| 157 | STAG1 | 5 (10%) |
| 158 | STAG2 | 5 (10%) |
| 159 | TUBG1 | 5 (10%) |
| 160 | VPS4B | 5 (10%) |
| 161 | WAPL | 5 (10%) |
| 162 | ZNF207 | 5 (10%) |
| 163 | ZWILCH | 5 (10%) |
| 164 | AC027237.1 | 4 (8%) |
| 165 | B9D2 | 4 (8%) |
| 166 | CCNF | 4 (8%) |
| 167 | CDK5RAP2 | 4 (8%) |
| 168 | CDT1 | 4 (8%) |
| 169 | CENPJ | 4 (8%) |
| 170 | CEP192 | 4 (8%) |
| 171 | CHMP1A | 4 (8%) |
| 172 | CKAP2 | 4 (8%) |
| 173 | DNAJC9 | 4 (8%) |
| 174 | DYNLL2 | 4 (8%) |
| 175 | E2F8 | 4 (8%) |
| 176 | GINS1 | 4 (8%) |
| 177 | HDAC8 | 4 (8%) |
| 178 | HSF1 | 4 (8%) |
| 179 | KIF14 | 4 (8%) |
| 180 | MCM7 | 4 (8%) |
| 181 | NIPBL | 4 (8%) |
| 182 | NUDC | 4 (8%) |
| 183 | NUMA1 | 4 (8%) |
| 184 | PCNT | 4 (8%) |
| 185 | PDS5A | 4 (8%) |
| 186 | PDS5B | 4 (8%) |
| 187 | PPP2R5B | 4 (8%) |
| 188 | PPP2R5E | 4 (8%) |
| 189 | RAD21 | 4 (8%) |
| 190 | RANBP2 | 4 (8%) |
| 191 | RB1 | 4 (8%) |
| 192 | RHOA | 4 (8%) |
| 193 | RPS27 | 4 (8%) |
| 194 | TACC1 | 4 (8%) |
| 195 | TACC3 | 4 (8%) |
| 196 | TAOK1 | 4 (8%) |
| 197 | TPR | 4 (8%) |
| 198 | TUBA1A | 4 (8%) |
| 199 | TUBA4A | 4 (8%) |
| 200 | TUBB4A | 4 (8%) |
| 201 | TUBB4B | 4 (8%) |
| 202 | TUBG2 | 4 (8%) |
| 203 | ABRAXAS2 | 3 (6%) |
| 204 | AKAP8 | 3 (6%) |
| 205 | AKAP9 | 3 (6%) |
| 206 | ANKRD53 | 3 (6%) |
| 207 | APC | 3 (6%) |
| 208 | BECN1 | 3 (6%) |
| 209 | BOD1 | 3 (6%) |
| 210 | CHAMP1 | 3 (6%) |
| 211 | CHEK2 | 3 (6%) |
| 212 | CHMP1B | 3 (6%) |
| 213 | CHMP2A | 3 (6%) |
| 214 | CHMP2B | 3 (6%) |
| 215 | CHMP4B | 3 (6%) |
| 216 | CHMP4C | 3 (6%) |
| 217 | CHMP5 | 3 (6%) |
| 218 | DCTN3 | 3 (6%) |
| 219 | EZH2 | 3 (6%) |
| 220 | GPSM2 | 3 (6%) |
| 221 | HELLS | 3 (6%) |
| 222 | KIF15 | 3 (6%) |
| 223 | KIF4B | 3 (6%) |
| 224 | KNSTRN | 3 (6%) |
| 225 | KPNB1 | 3 (6%) |
| 226 | LMNB1 | 3 (6%) |
| 227 | MAP10 | 3 (6%) |
| 228 | MSTO1 | 3 (6%) |
| 229 | MZT1 | 3 (6%) |
| 230 | NCAPD3 | 3 (6%) |
| 231 | NCAPH2 | 3 (6%) |
| 232 | PA2G4 | 3 (6%) |
| 233 | PAICS | 3 (6%) |
| 234 | PIBF1 | 3 (6%) |
| 235 | PIH1D1 | 3 (6%) |
| 236 | POC1A | 3 (6%) |
| 237 | PRKACA | 3 (6%) |
| 238 | PSRC1 | 3 (6%) |
| 239 | RAB11A | 3 (6%) |
| 240 | RAD51AP1 | 3 (6%) |
| 241 | SMARCA4 | 3 (6%) |
| 242 | SMARCB1 | 3 (6%) |
| 243 | STMN1 | 3 (6%) |
| 244 | TEX14 | 3 (6%) |
| 245 | TNKS | 3 (6%) |
| 246 | TUBA1B | 3 (6%) |
| 247 | TUBA1C | 3 (6%) |
| 248 | TUBA3C | 3 (6%) |
| 249 | TUBA3D | 3 (6%) |
| 250 | TUBA3E | 3 (6%) |
| 251 | TUBA4B | 3 (6%) |
| 252 | TUBA8 | 3 (6%) |
| 253 | TUBAL3 | 3 (6%) |
| 254 | TUBB1 | 3 (6%) |
| 255 | TUBB2A | 3 (6%) |
| 256 | TUBB2B | 3 (6%) |
| 257 | TUBB3 | 3 (6%) |
| 258 | TUBB6 | 3 (6%) |
| 259 | TUBB8 | 3 (6%) |
| 260 | ACTR1A | 2 (4%) |
| 261 | ADCY8 | 2 (4%) |
| 262 | AGER | 2 (4%) |
| 263 | AKAP8L | 2 (4%) |
| 264 | ALMS1 | 2 (4%) |
| 265 | ANAPC1 | 2 (4%) |
| 266 | ANAPC11 | 2 (4%) |
| 267 | ANAPC15 | 2 (4%) |
| 268 | ANAPC16 | 2 (4%) |
| 269 | ANAPC4 | 2 (4%) |
| 270 | ANAPC5 | 2 (4%) |
| 271 | ARHGEF10 | 2 (4%) |
| 272 | ATRX | 2 (4%) |
| 273 | BCCIP | 2 (4%) |
| 274 | BORA | 2 (4%) |
| 275 | CBX5 | 2 (4%) |
| 276 | CCP110 | 2 (4%) |
| 277 | CCSAP | 2 (4%) |
| 278 | CCT2 | 2 (4%) |
| 279 | CDC23 | 2 (4%) |
| 280 | CDC25B | 2 (4%) |
| 281 | CDC26 | 2 (4%) |
| 282 | CDC27 | 2 (4%) |
| 283 | CDC42 | 2 (4%) |
| 284 | CEP120 | 2 (4%) |
| 285 | CEP126 | 2 (4%) |
| 286 | CEP131 | 2 (4%) |
| 287 | CEP135 | 2 (4%) |
| 288 | CEP152 | 2 (4%) |
| 289 | CEP164 | 2 (4%) |
| 290 | CEP250 | 2 (4%) |
| 291 | CEP290 | 2 (4%) |
| 292 | CEP295 | 2 (4%) |
| 293 | CEP41 | 2 (4%) |
| 294 | CEP57 | 2 (4%) |
| 295 | CEP63 | 2 (4%) |
| 296 | CEP70 | 2 (4%) |
| 297 | CEP72 | 2 (4%) |
| 298 | CEP76 | 2 (4%) |
| 299 | CEP78 | 2 (4%) |
| 300 | CEP97 | 2 (4%) |
| 301 | CETN2 | 2 (4%) |
| 302 | CLTC | 2 (4%) |
| 303 | CNTRL | 2 (4%) |
| 304 | CSNK1D | 2 (4%) |
| 305 | CSNK1E | 2 (4%) |
| 306 | CUL3 | 2 (4%) |
| 307 | DAXX | 2 (4%) |
| 308 | DRG1 | 2 (4%) |
| 309 | DSCC1 | 2 (4%) |
| 310 | DTL | 2 (4%) |
| 311 | EFHC1 | 2 (4%) |
| 312 | EML1 | 2 (4%) |
| 313 | FGFR1OP | 2 (4%) |
| 314 | FLNA | 2 (4%) |
| 315 | FSD1 | 2 (4%) |
| 316 | GNAI1 | 2 (4%) |
| 317 | GOLGA2 | 2 (4%) |
| 318 | H2AFY | 2 (4%) |
| 319 | HAUS1 | 2 (4%) |
| 320 | HAUS2 | 2 (4%) |
| 321 | HAUS3 | 2 (4%) |
| 322 | HAUS4 | 2 (4%) |
| 323 | HAUS5 | 2 (4%) |
| 324 | HAUS6 | 2 (4%) |
| 325 | HAUS7 | 2 (4%) |
| 326 | HAUS8 | 2 (4%) |
| 327 | HIRA | 2 (4%) |
| 328 | HNRNPAB | 2 (4%) |
| 329 | HSP90AA1 | 2 (4%) |
| 330 | HSPA1A | 2 (4%) |
| 331 | HSPA1B | 2 (4%) |
| 332 | INTS13 | 2 (4%) |
| 333 | KIF22 | 2 (4%) |
| 334 | KIF3B | 2 (4%) |
| 335 | LCMT1 | 2 (4%) |
| 336 | MAP4 | 2 (4%) |
| 337 | MAP9 | 2 (4%) |
| 338 | MCM5 | 2 (4%) |
| 339 | MECP2 | 2 (4%) |
| 340 | MEIKIN | 2 (4%) |
| 341 | MLXIPL | 2 (4%) |
| 342 | NASP | 2 (4%) |
| 343 | NEDD1 | 2 (4%) |
| 344 | NEK6 | 2 (4%) |
| 345 | NFKBIA | 2 (4%) |
| 346 | NINL | 2 (4%) |
| 347 | ODF2 | 2 (4%) |
| 348 | OFD1 | 2 (4%) |
| 349 | OIP5 | 2 (4%) |
| 350 | PARP3 | 2 (4%) |
| 351 | PCM1 | 2 (4%) |
| 352 | PHF13 | 2 (4%) |
| 353 | PHF23 | 2 (4%) |
| 354 | PINX1 | 2 (4%) |
| 355 | PKD1 | 2 (4%) |
| 356 | PLK2 | 2 (4%) |
| 357 | POLD1 | 2 (4%) |
| 358 | PRKAR2B | 2 (4%) |
| 359 | PRKN | 2 (4%) |
| 360 | PTPA | 2 (4%) |
| 361 | RAE1 | 2 (4%) |
| 362 | RASA1 | 2 (4%) |
| 363 | RCC1 | 2 (4%) |
| 364 | SBDS | 2 (4%) |
| 365 | SDCCAG8 | 2 (4%) |
| 366 | SETD2 | 2 (4%) |
| 367 | SFI1 | 2 (4%) |
| 368 | SIRT2 | 2 (4%) |
| 369 | SLBP | 2 (4%) |
| 370 | SPAST | 2 (4%) |
| 371 | SPICE1 | 2 (4%) |
| 372 | SPOUT1 | 2 (4%) |
| 373 | SSNA1 | 2 (4%) |
| 374 | STIL | 2 (4%) |
| 375 | SUN2 | 2 (4%) |
| 376 | TACC2 | 2 (4%) |
| 377 | TBCE | 2 (4%) |
| 378 | TCP1 | 2 (4%) |
| 379 | TENT4A | 2 (4%) |
| 380 | TTN | 2 (4%) |
| 381 | TUBB | 2 (4%) |
| 382 | UBE2D4 | 2 (4%) |
| 383 | UBE2T | 2 (4%) |
| 384 | UVRAG | 2 (4%) |
| 385 | VCP | 2 (4%) |
| 386 | VRK1 | 2 (4%) |
| 387 | WDR62 | 2 (4%) |
| 388 | WRAP73 | 2 (4%) |
| 389 | YWHAE | 2 (4%) |
| 390 | YWHAG | 2 (4%) |
| 391 | ACTB | 1 (2%) |
| 392 | ACTG1 | 1 (2%) |
| 393 | AIFM1 | 1 (2%) |
| 394 | AJUBA | 1 (2%) |
| 395 | AKT1 | 1 (2%) |
| 396 | ALB | 1 (2%) |
| 397 | ANAPC10 | 1 (2%) |
| 398 | ANAPC2 | 1 (2%) |
| 399 | ANAPC7 | 1 (2%) |
| 400 | APAF1 | 1 (2%) |
| 401 | ARHGAP21 | 1 (2%) |
| 402 | ARHGEF2 | 1 (2%) |
| 403 | ARHGEF7 | 1 (2%) |
| 404 | ASF1A | 1 (2%) |
| 405 | ATM | 1 (2%) |
| 406 | ATP2A1 | 1 (2%) |
| 407 | AURKAIP1 | 1 (2%) |
| 408 | BAX | 1 (2%) |
| 409 | BAZ1B | 1 (2%) |
| 410 | BAZ2A | 1 (2%) |
| 411 | BLM | 1 (2%) |
| 412 | BOD1L2 | 1 (2%) |
| 413 | BRCA1 | 1 (2%) |
| 414 | CBX1 | 1 (2%) |
| 415 | CBX3 | 1 (2%) |
| 416 | CCNA1 | 1 (2%) |
| 417 | CCNH | 1 (2%) |
| 418 | CDC16 | 1 (2%) |
| 419 | CDC25A | 1 (2%) |
| 420 | CDC25C | 1 (2%) |
| 421 | CDC6 | 1 (2%) |
| 422 | CDK2 | 1 (2%) |
| 423 | CDK7 | 1 (2%) |
| 424 | CEBPB | 1 (2%) |
| 425 | CENPB | 1 (2%) |
| 426 | CENPV | 1 (2%) |
| 427 | CENPW | 1 (2%) |
| 428 | CENPX | 1 (2%) |
| 429 | CEP55 | 1 (2%) |
| 430 | CFDP1 | 1 (2%) |
| 431 | CHAF1B | 1 (2%) |
| 432 | CHMP4A | 1 (2%) |
| 433 | CHMP6 | 1 (2%) |
| 434 | CHMP7 | 1 (2%) |
| 435 | CHUK | 1 (2%) |
| 436 | CIDEA | 1 (2%) |
| 437 | CLEC7A | 1 (2%) |
| 438 | COLEC12 | 1 (2%) |
| 439 | CPEB1 | 1 (2%) |
| 440 | CSE1L | 1 (2%) |
| 441 | CSNK1A1 | 1 (2%) |
| 442 | CSNK2A1 | 1 (2%) |
| 443 | CSNK2A2 | 1 (2%) |
| 444 | CSNK2B | 1 (2%) |
| 445 | CTCF | 1 (2%) |
| 446 | CYP1A1 | 1 (2%) |
| 447 | DAAM1 | 1 (2%) |
| 448 | DBF4 | 1 (2%) |
| 449 | DCTN4 | 1 (2%) |
| 450 | DCTN5 | 1 (2%) |
| 451 | DCTN6 | 1 (2%) |
| 452 | DDC | 1 (2%) |
| 453 | DDX39B | 1 (2%) |
| 454 | DERPC | 1 (2%) |
| 455 | DES | 1 (2%) |
| 456 | DFFA | 1 (2%) |
| 457 | DIAPH1 | 1 (2%) |
| 458 | DIAPH2 | 1 (2%) |
| 459 | DIAPH3 | 1 (2%) |
| 460 | DIS3L2 | 1 (2%) |
| 461 | DNMT1 | 1 (2%) |
| 462 | DNMT3A | 1 (2%) |
| 463 | DPPA3 | 1 (2%) |
| 464 | DTYMK | 1 (2%) |
| 465 | DUSP1 | 1 (2%) |
| 466 | DVL1 | 1 (2%) |
| 467 | DVL2 | 1 (2%) |
| 468 | DVL3 | 1 (2%) |
| 469 | DYNLT1 | 1 (2%) |
| 470 | DYNLT3 | 1 (2%) |
| 471 | ECT2 | 1 (2%) |
| 472 | EED | 1 (2%) |
| 473 | ENDOG | 1 (2%) |
| 474 | ESCO2 | 1 (2%) |
| 475 | EVI5 | 1 (2%) |
| 476 | EVL | 1 (2%) |
| 477 | FBXO28 | 1 (2%) |
| 478 | FBXO5 | 1 (2%) |
| 479 | FECH | 1 (2%) |
| 480 | FGF10 | 1 (2%) |
| 481 | FMNL1 | 1 (2%) |
| 482 | FMNL2 | 1 (2%) |
| 483 | FMNL3 | 1 (2%) |
| 484 | FMR1 | 1 (2%) |
| 485 | FZR1 | 1 (2%) |
| 486 | GADD45A | 1 (2%) |
| 487 | GCOM2 | 1 (2%) |
| 488 | GEN1 | 1 (2%) |
| 489 | GIT1 | 1 (2%) |
| 490 | GPATCH11 | 1 (2%) |
| 491 | GSK3B | 1 (2%) |
| 492 | HASPIN | 1 (2%) |
| 493 | HAT1 | 1 (2%) |
| 494 | HECW2 | 1 (2%) |
| 495 | HNRNPA1 | 1 (2%) |
| 496 | HNRNPL | 1 (2%) |
| 497 | HTT | 1 (2%) |
| 498 | IK | 1 (2%) |
| 499 | IKBKB | 1 (2%) |
| 500 | IKBKG | 1 (2%) |
| 501 | IKZF1 | 1 (2%) |
| 502 | INO80 | 1 (2%) |
| 503 | ITCH | 1 (2%) |
| 504 | ITGB1 | 1 (2%) |
| 505 | KANSL1 | 1 (2%) |
| 506 | KAT2B | 1 (2%) |
| 507 | KAT8 | 1 (2%) |
| 508 | KATNB1 | 1 (2%) |
| 509 | KDM4A | 1 (2%) |
| 510 | KDM4C | 1 (2%) |
| 511 | KDM4D | 1 (2%) |
| 512 | KIF1B | 1 (2%) |
| 513 | KIF25 | 1 (2%) |
| 514 | KLHL13 | 1 (2%) |
| 515 | KLHL22 | 1 (2%) |
| 516 | KLHL9 | 1 (2%) |
| 517 | KMT5B | 1 (2%) |
| 518 | KMT5C | 1 (2%) |
| 519 | LRWD1 | 1 (2%) |
| 520 | MAD2L2 | 1 (2%) |
| 521 | MAPK13 | 1 (2%) |
| 522 | MAU2 | 1 (2%) |
| 523 | MCM10 | 1 (2%) |
| 524 | MCPH1 | 1 (2%) |
| 525 | MDM2 | 1 (2%) |
| 526 | MEAF6 | 1 (2%) |
| 527 | METAP2 | 1 (2%) |
| 528 | MIS18A | 1 (2%) |
| 529 | MIS18BP1 | 1 (2%) |
| 530 | MLH1 | 1 (2%) |
| 531 | MNAT1 | 1 (2%) |
| 532 | MRPL35 | 1 (2%) |
| 533 | MRPL39 | 1 (2%) |
| 534 | MRTFA | 1 (2%) |
| 535 | MSH2 | 1 (2%) |
| 536 | MTBP | 1 (2%) |
| 537 | MYLK | 1 (2%) |
| 538 | MZT2A | 1 (2%) |
| 539 | MZT2B | 1 (2%) |
| 540 | NAA10 | 1 (2%) |
| 541 | NAA50 | 1 (2%) |
| 542 | NCL | 1 (2%) |
| 543 | NEFL | 1 (2%) |
| 544 | NEK7 | 1 (2%) |
| 545 | NEK9 | 1 (2%) |
| 546 | NFKB1 | 1 (2%) |
| 547 | NFKB2 | 1 (2%) |
| 548 | NGDN | 1 (2%) |
| 549 | NME7 | 1 (2%) |
| 550 | NMI | 1 (2%) |
| 551 | NMNAT1 | 1 (2%) |
| 552 | NPM1 | 1 (2%) |
| 553 | NSFL1C | 1 (2%) |
| 554 | NSMCE2 | 1 (2%) |
| 555 | NSUN2 | 1 (2%) |
| 556 | NUDCD2 | 1 (2%) |
| 557 | NUP205 | 1 (2%) |
| 558 | OAZ1 | 1 (2%) |
| 559 | ORC2 | 1 (2%) |
| 560 | PAK1 | 1 (2%) |
| 561 | PAM16 | 1 (2%) |
| 562 | PAX6 | 1 (2%) |
| 563 | PAXIP1 | 1 (2%) |
| 564 | PBK | 1 (2%) |
| 565 | PCID2 | 1 (2%) |
| 566 | PEBP1 | 1 (2%) |
| 567 | PFN1 | 1 (2%) |
| 568 | PFN2 | 1 (2%) |
| 569 | PHF2 | 1 (2%) |
| 570 | PHF6 | 1 (2%) |
| 571 | PKHD1 | 1 (2%) |
| 572 | PKMYT1 | 1 (2%) |
| 573 | PLCZ1 | 1 (2%) |
| 574 | POGZ | 1 (2%) |
| 575 | POLE2 | 1 (2%) |
| 576 | POLR2M | 1 (2%) |
| 577 | PPME1 | 1 (2%) |
| 578 | PPP1R10 | 1 (2%) |
| 579 | PPP1R12A | 1 (2%) |
| 580 | PPP2R2A | 1 (2%) |
| 581 | PPP2R3B | 1 (2%) |
| 582 | PSEN1 | 1 (2%) |
| 583 | PSEN2 | 1 (2%) |
| 584 | PSMA3 | 1 (2%) |
| 585 | PSMG2 | 1 (2%) |
| 586 | PTTG2 | 1 (2%) |
| 587 | PTTG3P | 1 (2%) |
| 588 | RAC1 | 1 (2%) |
| 589 | RAD54L | 1 (2%) |
| 590 | RANBP1 | 1 (2%) |
| 591 | RASSF2 | 1 (2%) |
| 592 | REC8 | 1 (2%) |
| 593 | RECQL5 | 1 (2%) |
| 594 | RELA | 1 (2%) |
| 595 | RHOB | 1 (2%) |
| 596 | RHOC | 1 (2%) |
| 597 | RHOD | 1 (2%) |
| 598 | RIF1 | 1 (2%) |
| 599 | RIOK2 | 1 (2%) |
| 600 | RNF186 | 1 (2%) |
| 601 | RNF6 | 1 (2%) |
| 602 | ROCK2 | 1 (2%) |
| 603 | RPA3 | 1 (2%) |
| 604 | RRN3 | 1 (2%) |
| 605 | RRS1 | 1 (2%) |
| 606 | SAPCD2 | 1 (2%) |
| 607 | SCAI | 1 (2%) |
| 608 | SCN1B | 1 (2%) |
| 609 | SCN2B | 1 (2%) |
| 610 | SEPT1 | 1 (2%) |
| 611 | SEPT2 | 1 (2%) |
| 612 | SEPT6 | 1 (2%) |
| 613 | SEPT7 | 1 (2%) |
| 614 | SIN3A | 1 (2%) |
| 615 | SKA3 | 1 (2%) |
| 616 | SLC2A1 | 1 (2%) |
| 617 | SLF1 | 1 (2%) |
| 618 | SLF2 | 1 (2%) |
| 619 | SMC1B | 1 (2%) |
| 620 | SMC5 | 1 (2%) |
| 621 | SNAI1 | 1 (2%) |
| 622 | SNRPD1 | 1 (2%) |
| 623 | SPRY1 | 1 (2%) |
| 624 | SPRY2 | 1 (2%) |
| 625 | SRC | 1 (2%) |
| 626 | SRF | 1 (2%) |
| 627 | SRGAP2 | 1 (2%) |
| 628 | SS18L1 | 1 (2%) |
| 629 | ST20 | 1 (2%) |
| 630 | STAG3 | 1 (2%) |
| 631 | STAR | 1 (2%) |
| 632 | STPG4 | 1 (2%) |
| 633 | SUGT1 | 1 (2%) |
| 634 | SUMO3 | 1 (2%) |
| 635 | SUV39H1 | 1 (2%) |
| 636 | SUV39H2 | 1 (2%) |
| 637 | SYCP1 | 1 (2%) |
| 638 | SYCP2L | 1 (2%) |
| 639 | SYCP3 | 1 (2%) |
| 640 | TAF1B | 1 (2%) |
| 641 | TAF1C | 1 (2%) |
| 642 | TBCCD1 | 1 (2%) |
| 643 | TBK1 | 1 (2%) |
| 644 | TBP | 1 (2%) |
| 645 | TDRD7 | 1 (2%) |
| 646 | TET3 | 1 (2%) |
| 647 | TH | 1 (2%) |
| 648 | THOC1 | 1 (2%) |
| 649 | THOC5 | 1 (2%) |
| 650 | TICAM1 | 1 (2%) |
| 651 | TLR3 | 1 (2%) |
| 652 | TLR4 | 1 (2%) |
| 653 | TLR7 | 1 (2%) |
| 654 | TLR8 | 1 (2%) |
| 655 | TLR9 | 1 (2%) |
| 656 | TP53 | 1 (2%) |
| 657 | TP53BP1 | 1 (2%) |
| 658 | TRAPPC12 | 1 (2%) |
| 659 | TRIP13 | 1 (2%) |
| 660 | TUBGCP2 | 1 (2%) |
| 661 | TUBGCP3 | 1 (2%) |
| 662 | TUBGCP4 | 1 (2%) |
| 663 | TUBGCP5 | 1 (2%) |
| 664 | TUBGCP6 | 1 (2%) |
| 665 | UBE2D1 | 1 (2%) |
| 666 | UBE2E1 | 1 (2%) |
| 667 | UBTF | 1 (2%) |
| 668 | UBXN2B | 1 (2%) |
| 669 | USP44 | 1 (2%) |
| 670 | VIM | 1 (2%) |
| 671 | VPS4A | 1 (2%) |
| 672 | WEE1 | 1 (2%) |
| 673 | XRCC3 | 1 (2%) |
| 674 | ZC3H12A | 1 (2%) |
| 675 | ZNF276 | 1 (2%) |
| 676 | ZNF330 | 1 (2%) |
| 677 | ZNFX1 | 1 (2%) |

TABLE 2
Genes contained in each of the 51 gene sets as selected by mean abiraterone and
onvansertib significance score and associated with response to the combination.

| GENE SET | GENES |
|---|---|
| GNF2_BUB1 | ASF1B, ASPM, AURKB, BUB1, BUB1B, CCNB2, CDC20, CDCA8, CDT1, CENPE, CHAF1B, |
| | CKS1B, ESPL1, FOXM1, GMNN, HMMR, KIF20A, KIF2C, MCM4, MELK, NDC80, PLK1, |
| | RANBP1, RRM2, SMC2, SPAG5 |
| GNF2_BUB1B | ASF1B, ASPM, AURKA, AURKB, BUB1, BUB1B, CCNA2, CCNB2, CDC20, CDCA8, CENPM, |
| | CEP55, CSE1L, ESPL1, FANCI, GINS1, GMNN, HELLS, HJURP, HMMR, KIF11, KIF14, KIF20A, |
| | LMNB1, MCM2, MCM3, MCM4, MELK, MRPL39, NASP, NCAPD2, NDC80, NUP205, PA2G4, |
| | PAICS, PCNA, PLK1, POLD1, RACGAP1, RRM1, RRM2, SMC4, SPAG5, TCP1, TOP2A, TTK, |
| | TYMS, UBE2C, ZWINT |
| GNF2_CCNA2 | ASF1B, ASPM, AURKA, AURKB, BIRC5, BUB1B, CCNA2, CCNB2, CDC20, CDCA3, CDCA8, |
| | CDK1, CENPA, CENPE, CENPF, CKS1B, CKS2, DLGAP5, E2F8, ESPL1, EZH2, FANCI, FEN1, |
| | FOXM1, GINS2, GMNN, H2AFX, HJURP, HMGB2, HMMR, KIF11, KIF14, KIF18B, KIF20A, |
| | KIF4A, MCM2, MCM3, MCM4, MCM6, MCM7, MELK, MKI67, MT1JP, NCAPD2, NCAPG, |
| | NDC80, NUSAP1, PCLAF, PCNA, PLK1, PRC1, PRIM1, RACGAP1, RAD51AP1, RFC3, RFC4, |
| | RRM1, RRM2, SHCBP1, SMC2, SMC4, TMPO, TOP2A, TPX2, TTK, TYMS, UBE2C, ZWINT |
| GNF2_CCNB2 | ASPM, AURKA, BIRC5, BUB1, BUB1B, CCNA2, CCNB2, CDC20, CDCA3, CDCA8, CDK1, |
| | CENPE, CENPF, CKAP2, CKS2, DLGAP5, ESPL1, FEN1, FOXM1, GMNN, H2AFX, HMGB2, |
| | HMMR, KIF11, KIF18B, KIF20A, KIF2C, KIF4A, MCM2, MCM4, MELK, MT1JP, NCAPH, |
| | NDC80, NSD2, NUSAP1, OIP5, PBK, PCNA, PLK4, PRC1, PTTG1, RACGAP1, RFC4, RRM1, |
| | RRM2, SHCBP1, SMC2, SMC4, SPAG5, TOP2A, TPX2, TTK, TYMS, UBE2C, UBE2S, ZWINT |
| GNF2_CDC2 | ASF1A, ASPM, AURKA, BIRC5, CCNA2, CCNB2, CCNF, CDC20, CDCA3, CDCA8, CDK1, CENPE, |
| | CENPF, CKAP2, CKS2, DLGAP5, DNAJC9, E2F8, EZH2, FANCI, FEN1, FOXM1, GINS2, H2AFX, |
| | HJURP, HMGB2, HMMR, KIF11, KIF15, KIF18B, KIF20A, KIF4A, KIFC1, MCM2, MELK, MKI67, |
| | MT1JP, MYBL2, NCAPG, NDC80, NSD2, NUSAP1, PCNA, PLK4, PRC1, PRIM1, PTTG1, RFC3, |
| | RFC4, RRM1, RRM2, SHCBP1, SMC2, SMC4, TMPO, TOP2A, TPX2, TTK, TYMS, UBE2C, |
| | UBE2S, ZWINT |
| GNF2_CDC20 | ASPM, AURKA, AURKB, BIRC5, BUB1, BUB1B, CCNA2, CCNB2, CDC20, CDCA3, CDCA8, |
| | CDK1, CENPE, CENPF, CKS1B, CKS2, DLGAP5, ESPL1, FOXM1, GMNN, H2AFX, HMMR, |
| | KIF11, KIF18B, KIF20A, KIF2C, KIF4A, MCM2, MCM4, MELK, MKI67, MT1JP, NCAPH, |
| | NDC80, NSD2, NUSAP1, PCNA, PLK1, PLK4, PRC1, PTTG1, RACGAP1, RAN, RRM1, RRM2, |
| | SHCBP1, SMC2, SMC4, SPAG5, TOP2A, TPX2, TTK, TYMS, UBE2C, UBE2S, ZWINT |
| GNF2_CENPE | ASPM, AURKA, AURKB, BIRC5, BUB1, CCNA2, CCNB2, CCNF, CDC20, CDCA3, CDCA8, CDK1, |
| | CENPE, CENPF, CKS1B, CKS2, DLGAP5, ESPL1, FOXM1, H2AFX, HMMR, KIF11, KIF18B, |
| | MKI67, MT1JP, MYBL2, NDC80, NSD2, NUSAP1, PCNA, PRC1, PRIM1, RFC3, RRM1, RRM2, |
| | SHCBP1, SMC2, TMPO, TYMS, UBE2C, ZWINT |
| GNF2_CENPF | ASPM, AURKA, BIRC5, CCNA2, CCNB2, CCNF, CDC20, CDCA3, CDCA8, CDK1, CENPE, CENPF, |
| | CKAP2, CKS2, DLGAP5, DNAJC9, DTYMK, E2F8, FANCI, FEN1, FOXM1, GINS2, H2AFX, HAT1, |
| | HJURP, HMGB2, HMMR, KIF11, KIF15, KIF18B, KIF4A, KIFC1, MCM10, MCM2, MCM3, |
| | MCM7, MKI67, MT1JP, MYBL2, NDC80, NSD2, NUSAP1, PCLAF, PCNA, PLK4, PRC1, PRIM1, |
| | PTTG1, RFC4, RRM1, RRM2, SHCBP1, SMC2, SMC4, TMPO, TOP2A, TPX2, TTK, TYMS, |
| | UBE2C, UBE2S, ZWINT |
| GNF2_CKS2 | ASPM, BIRC5, CCNA2, CCNB2, CCT2, CDC20, CDCA3, CDK1, CENPE, CENPF, CKAP2, CKS2, |
| | DLGAP5, DTL, FANCI, FEN1, FOXM1, GINS1, GINS2, GMNN, HMGB2, HMMR, HNRNPAB, |
| | KIF20A, MCM2, MCM4, METAP2, MRPL35, NDC80, NUSAP1, PCLAF, PCNA, PLK4, PRC1, |
| | PTTG1, RACGAP1, RFC3, RFC4, RPA3, RRM1, SHCBP1, SLBP, SMC2, SMC4, SNRPD1, TOP2A, |
| | TTK, TYMS, VRK1, ZWINT |
| GNF2_ESPL1 | ASF1B, ASPM, AURKB, BIRC5, BUB1, BUB1B, CCNA2, CCNB2, CDC20, CDCA8, CENPA, |
| | CENPE, CENPM, CKS1B, ESPL1, FOXM1, HMMR, KIF11, KIF20A, KIF2C, MAD2L1, MCM4, |
| | MCM6, MELK, MKI67, NCAPD2, PAICS, PCNA, PLK1, RRM2, SMC2, SPAG5, TOP2A, TPX2, |
| | UBE2C |
| GNF2_H2AFX | ASPM, AURKA, BIRC5, CCNA2, CCNB2, CCNF, CDC20, CDCA8, CDK1, CENPE, CENPF, |
| | DLGAP5, DNAJC9, FOXM1, H2AFX, HMGB2, KIF18B, MCM2, NSD2, NUSAP1, PCNA, PRC1, |
| | PTTG1, RRM1, RRM2, SHCBP1, SMC4, TMPO, TYMS, UBE2S, ZWINT |
| GNF2_HMMR | ASPM, AURKA, AURKB, BIRC5, BUB1, BUB1B, CCNA2, CCNB2, CDC20, CDCA3, CDCA8, |
| | CDK1, CENPE, CENPF, CKS2, DLGAP5, E2F8, ESPL1, FEN1, FOXM1, GINS2, GMNN, HMMR, |
| | KIF11, KIF18B, KIF20A, MCM2, MCM4, MELK, MKI67, NDC80, NUSAP1, PCNA, PLK1, PLK4, |
| | PRC1, RFC3, RRM1, RRM2, SHCBP1, SMC2, SMC4, TOP2A, TTK, TYMS, UBE2C, ZWINT |
| GNF2_MCM4 | ASF1B, ASPM, AURKA, AURKB, BUB1, BUB1B, CCNA2, CCNB2, CDC20, CDCA8, CENPM, |
| | CKS2, ESPL1, FANCI, FEN1, FOXM1, GINS1, GINS2, GMNN, HELLS, HMMR, KIF11, KIF14, |
| | KIF18B, LMNB1, MAD2L1, MCM2, MCM3, MCM4, MCM5, MCM6, MCM7, MELK, NASP, |
| | NDC80, PA2G4, PAICS, PCLAF, PCNA, POLD1, RACGAP1, RAD51AP1, RFC3, RFC4, RRM1, |
| | RRM2, SMC4, TCP1, TOP2A, TTK, TYMS, UBE2C, ZWINT |
| GNF2_MKI67 | ASPM, AURKA, BIRC5, CCNA2, CDC20, CDK1, CENPA, CENPE, CENPF, ESPL1, FOXM1, |
| | HMMR, KIF11, KIF18B, KIF20A, MELK, MKI67, MT1JP, NCAPD2, NCAPG, NUSAP1, PCNA, |
| | RRM1, RRM2, TOP2A, TPX2, UBE2C, ZWINT |
| GNF2_PCNA | ASPM, AURKA, BIRC5, BUB1B, CCNA2, CCNB2, CDC20, CDCA8, CDK1, CENPE, CENPF, CKS2, |
| | DLGAP5, DNAJC9, DTL, ESPL1, FANCI, FEN1, FOXM1, GINS1, GINS2, GMNN, H2AFX, |
| | HMGB2, HMMR, HNRNPAB, KIF11, KIF15, KIF18B, MCM2, MCM3, MCM4, MCM5, MCM6, |
| | MCM7, MELK, MKI67, MSH2, MT1JP, MYBL2, NDC80, NSD2, NUSAP1, PA2G4, PAXIP1, |
| | PCLAF, PCNA, PLK4, POLE2, PRC1, PRIM1, RACGAP1, RAD51AP1, RAD54L, RFC4, RRM1, |
| | RRM2, SHCBP1, SLBP, SMC2, SMC4, TMPO, TOP2A, TPX2, TTK, TYMS, VRK1, ZWINT |
| GNF2_RRM2 | ASPM, AURKA, AURKB, BIRC5, BUB1, BUB1B, CCNA2, CCNB2, CDC20, CDCA8, CDK1, |
| | CENPE, CENPF, CENPM, DLGAP5, ESPL1, FOXM1, GMNN, H2AFX, HMMR, KIF11, KIF18B, |
| | LMNB1, MCM2, MCM3, MCM4, MCM6, MELK, MKI67, NDC80, NUSAP1, PCNA, PLK1, |
| | RRM1, RRM2, SMC1A, TOP2A, TYMS, UBE2C, ZWINT |
| GNF2_SMC2L1 | ASPM, BIRC5, BUB1, CCNA2, CCNB2, CDC20, CDCA8, CDK1, CENPE, CENPF, CKS1B, CKS2, |
| | ESPL1, FOXM1, HMMR, KIF11, KIF18B, MCM3, MCM6, MT1JP, MYBL2, NDC80, NUSAP1, |
| | PCNA, PRIM1, RFC3, RFC4, RRM1, SMC2, SMC4, TOP2A, TTK, ZWINT |
| GNF2_TTK | ASPM, BUB1B, CCNA2, CCNB2, CCT2, CDC20, CDK1, CENPF, CENPU, CKS2, DBF4, FANCI, |
| | FEN1, FOXM1, GINS2, GMNN, HJURP, HMMR, KIF11, KIF18B, KIF2C, MCM2, MCM4, MELK, |
| | NDC80, NUSAP1, PCNA, PLK4, PRC1, RACGAP1, RFC4, RRM1, SMC2, SMC4, TOP2A, TPX2, |
| | TTK, TYMS, ZWINT |
| GO_ATTACHMENT_{—} | ABRAXAS2, APC, AURKB, AURKC, BECN1, BOD1, BUB3, CCNB1, CDC42, CDT1, CENPC, |
| OF_SPINDLE_{—} | CENPE, CHAMP1, ECT2, HNRNPU, KIF2C, KNL1, KNSTRN, MAD1L1, MIS12, NDC80, NEK2, |
| MICROTUBULES_{—} | NUF2, RACGAP1, RB1, RCC2, SEH1L, SGO1, SIRT2, SPAG5, TEX14, ZNF207 |
| TO_KINETOCHORE | |
| GO_CARBOHYDRATE_{—} | ADCY8, AGER, CLEC7A, COLEC12, MLXIPL, PIH1D1, SMARCA4, SMARCB1 |
| MEDIATED_{—} | |
| SIGNALING | |
| GO_CHROMOSOME_{—} | AHCTF1, ANAPC16, APC, ATRX, AURKA, AURKB, AURKC, BAZ1B, BIRC5, BOD1, BOD1L2, |
| CENTROMERIC_{—} | BUB1, BUB1B, BUB3, CBX1, CBX3, CBX5, CCNB1, CDCA5, CDCA8, CDT1, CEBPB, CENPA, |
| REGION | CENPB, CENPC, CENPE, CENPF, CENPH, CENPI, CENPK, CENPL, CENPM, CENPN, CENPO, |
| | CENPP, CENPQ, CENPS, CENPT, CENPU, CENPV, CENPW, CENPX, CFDP1, CHAMP1, CKAP5, |
| | CLASP1, CLASP2, CLIP1, CSNK1A1, CTCF, DAXX, DCTN1, DCTN2, DCTN3, DCTN4, DCTN5, |
| | DCTN6, DNMT1, DNMT3A, DSCC1, DSN1, DYNC1I1, DYNC1LI1, DYNC1LI2, DYNLL1, |
| | DYNLT3, ERCC6L, ESCO2, FBXO28, FMR1, GPATCH11, H2AFY, HELLS, HIRA, HJURP, |
| | HNRNPU, HSF1, IKZF1, INCENP, ITGB3BP, KANSL1, KAT2B, KAT8, KDM4A, KDM4C, KDM4D, |
| | KIF18A, KIF22, KIF2B, KIF2C, KMT5B, KMT5C, KNL1, KNSTRN, KNTC1, LRWD1, MAD1L1, |
| | MAD2L1, MEAF6, MEIKIN, MIS12, MIS18A, MIS18BP1, MTBP, NCAPD2, NCAPD3, NCAPG, |
| | NDC80, NDE1, NDEL1, NEK2, NGDN, NSL1, NUDCD2, NUF2, NUP107, NUP133, NUP160, |
| | NUP37, NUP43, NUP85, NUP98, OIP5, ORC2, PAFAH1B1, PDS5A, PDS5B, PHF2, PHF6, |
| | PINX1, PKHD1, PLK1, PMF1, PPP1CC, PPP1R12A, PPP2CA, PPP2CB, PPP2R1A, PPP2R5A, |
| | PPP2R5C, PSEN1, PSEN2, RAD21, RANGAP1, RASSF2, RCC2, REC8, SEC13, SEH1L, SEPT2, |
| | SEPT6, SEPT7, SGO1, SGO2, SIN3A, SKA1, SKA2, SKA3, SMC1A, SMC1B, SMC3, SNAI1, |
| | SPAG5, SPC24, SPC25, SPDL1, SPOUT1, SS18L1, STAG1, STAG2, STAG3, SUGT1, SUMO3, |
| | SUV39H1, SUV39H2, SYCP1, SYCP2L, SYCP3, TEX14, TP53BP1, TPR, TRAPPC12, TTK, |
| | UVRAG, WAPL, XPO1, ZNF207, ZNF276, ZNF330, ZNFX1, ZW10, ZWILCH, ZWINT |
| GO_FEMALE_{—} | MEIKIN, MLH1, NCAPH, NCAPH2, PLK1, TTK |
| MEIOSIS_{—} | |
| CHROMOSOME_{—} | |
| SEGREGATION | |
| GO_INTERFERON_{—} | NMI, TBK1, TICAM1, TLR3, TLR4, TLR7, TLR8, TLR9 |
| BETA_BIOSYNTHETIC_{—} | |
| PROCESS | |
| GO_MAINTENANCE_{—} | AKAP9, ALB, ARHGAP21, ASPM, ATP2A1, GCOM2, GPSM2, PAFAH1B1, POLR2M, SPOUT1, |
| OF_ORGANELLE_{—} | TBCCD1, UVRAG |
| LOCATION | |
| GO_MICROTUBULE_{—} | ABRAXAS2, AC027237.1, ANKRD53, ARHGEF10, ARHGEF2, AURKA, AURKB, AURKC, BCCIP, |
| CYTOSKELETON_{—} | BIRC5, BORA, CCNB1, CCSAP, CDC20, CDK5RAP2, CENPA, CENPE, CEP126, CEP192, CEP97, |
| ORGANIZATION_{—} | CHEK2, CHMP1A, CHMP1B, CHMP2A, CHMP2B, CHMP4B, CHMP4C, CHMP5, CLASP1, |
| INVOLVED_IN_{—} | CLASP2, CLTC, DCTN1, DCTN2, DRG1, DYNC1H1, DYNLT1, EFHC1, EML1, ESPL1, FGF10, |
| MITOSIS | FLNA, FSD1, GNAI1, GOLGA2, GPSM2, HNRNPU, HSPA1A, HSPA1B, HTT, INTS13, KIF11, |
| | KIF2A, KIF3B, KIF4A, KIF4B, KIFC1, KPNB1, MAP10, MAP4, MAP9, MCPH1, MECP2, MSTO1, |
| | MYBL2, MZT1, NDC80, NDE1, NDEL1, NEK2, NSFL1C, NUF2, NUMA1, NUP62, NUSAP1, |
| | PAFAH1B1, PARP3, PAX6, PCNT, PIBF1, PKD1, PLK1, PLK2, POC1A, PRC1, PSRC1, PTPA, |
| | RAB11A, RACGAP1, RAE1, RAN, RCC1, RHOA, SAPCD2, SBDS, SETD2, SMC1A, SMC3, SPAST, |
| | SPC25, SPDL1, SPICE1, SPRY1, SPRY2, STAG1, STAG2, STIL, STMN1, SUN2, TACC1, TACC2, |
| | TACC3, TBCE, TNKS, TPR, TPX2, TTK, TUBG1, TUBG2, UBXN2B, VCP, VPS4B, WDR62, |
| | WRAP73, ZNF207, ZW10 |
| GO_MITOTIC_{—} | AKAP8, AKAP8L, CDCA5, CHMP1A, NCAPD2, NCAPD3, NCAPG, NCAPH, NCAPH2, NIPBL, |
| CHROMOSOME_{—} | NUSAP1, PHF13, PHF23, SMC2, SMC4, TENT4A, TTN |
| CONDENSATION | |
| GO_MITOTIC_{—} | AC027237.1, AKAP8, AKAP8L, ANAPC1, ANAPC11, ANAPC15, ANAPC4, ANAPC5, ANKRD53, |
| SISTER_{—} | APC, ATM, ATRX, AURKB, AURKC, BECN1, BOD1, BUB1, BUB1B, BUB3, CCNB1, CDC20, |
| CHROMATID_{—} | CDC23, CDC26, CDC27, CDC6, CDCA5, CDCA8, CDK5RAP2, CDT1, CENPC, CENPE, CENPF, |
| SEGREGATION | CHAMP1, CHMP1A, CHMP1B, CHMP2A, CHMP2B, CHMP4A, CHMP4B, CHMP4C, CHMP5, |
| | CHMP6, CHMP7, CUL3, DERPC, DIS3L2, DLGAP5, DSCC1, DSN1, DUSP1, DYNC1LI1, ESPL1, |
| | FBXO5, GEN1, H2AFY, HASPIN, HECW2, HIRA, HNRNPU, IK, INCENP, INO80, KATNB1, |
| | KIF14, KIF18A, KIF18B, KIF22, KIF25, KIF2C, KIF4A, KIF4B, KIFC1, KLHL22, KNSTRN, KPNB1, |
| | LCMT1, MAD1L1, MAD2L1, MAD2L2, MAP10, MAU2, MIS12, MSTO1, NAA10, NAA50, |
| | NCAPD2, NCAPD3, NCAPG, NCAPH, NCAPH2, NDC80, NEK2, NEK6, NIPBL, NSL1, NSMCE2, |
| | NUF2, NUMA1, NUP62, NUSAP1, PCID2, PDS5A, PDS5B, PHF13, PHF23, PIBF1, PINX1, |
| | PLK1, POGZ, PPP2R1A, PRC1, PSMG2, PSRC1, PTTG1, PTTG2, PTTG3P, RAB11A, RACGAP1, |
| | RAD21, RAN, RB1, RECQL5, RIOK2, RRS1, SEH1L, SGO1, SLF1, SLF2, SMC1A, SMC2, SMC4, |
| | SMC5, SPAG5, SPDL1, TACC3, TENT4A, TEX14, TNKS, TPR, TRIP13, TTK, TTN, TUBG1, |
| | TUBG2, USP44, VPS4A, VPS4B, XRCC3, ZNF207, ZW10, ZWINT |
| GO_MITOTIC_{—} | ABRAXAS2, AC027237.1, ANKRD53, ARHGEF10, AURKA, AURKB, AURKC, BCCIP, BIRC5, |
| SPINDLE_{—} | BORA, CCNB1, CCSAP, CDC20, CENPE, CEP126, CEP192, CEP97, CHEK2, CHMP1A, CHMP1B, |
| ORGANIZATION | CHMP2A, CHMP2B, CHMP4B, CHMP4C, CHMP5, CLASP1, CLASP2, CLTC, DCTN1, DCTN2, |
| | DRG1, DYNC1H1, EFHC1, EML1, FLNA, FSD1, GNAI1, GOLGA2, GPSM2, HNRNPU, HSPA1A, |
| | HSPA1B, INTS13, KIF11, KIF2A, KIF3B, KIF4A, KIF4B, KIFC1, KPNB1, MAP10, MAP4, MAP9, |
| | MECP2, MSTO1, MYBL2, MZT1, NDC80, NEK2, NUF2, NUMA1, NUP62, PARP3, PCNT, |
| | PIBF1, PKD1, PLK1, PLK2, POC1A, PRC1, PSRC1, PTPA, RAB11A, RACGAP1, RAE1, RAN, |
| | RCC1, RHOA, SBDS, SMC1A, SMC3, SPAST, SPC25, SPICE1, STAG1, STAG2, STIL, STMN1, |
| | SUN2, TACC1, TACC2, TACC3, TBCE, TNKS, TPR, TPX2, TTK, TUBG1, TUBG2, VCP, VPS4B, |
| | WDR62, WRAP73, ZNF207 |
| GO_NEGATIVE_{—} | CHEK2, DDX39B, PPP1R10, THOC1, THOC5 |
| REGULATION_{—} | |
| OF_DNA_DAMAGE_{—} | |
| CHECKPOINT | |
| GO_POSITIVE_{—} | BECN1, CCNB1, HNRNPU, RCC2, SIRT2 |
| REGULATION_{—} | |
| OF_ATTACHMENT_{—} | |
| OF_SPINDLE_{—} | |
| MICROTUBULES_{—} | |
| TO_KINETOCHORE | |
| GO_POSITIVE_{—} | CENPJ, CEP120, CEP295, NUP62, PLK4, VPS4B |
| REGULATION_{—} | |
| OF_CENTRIOLE_{—} | |
| REPLICATION | |
| GO_POSITIVE_{—} | CENPJ, CEP120, CEP295, NUP62, PLK4, POC1A, ROCK2, VPS4B |
| REGULATION_{—} | |
| OF_CENTROSOME_{—} | |
| DUPLICATION | |
| GO_PRONUCLEUS | AKAP8, AURKA, BLM, CCNA2, CENPF, DPPA3, EED, EZH2, HNRNPL, HSF1, PLCZ1, RIF1, |
| | SLC2A1, STPG4, TBP, TET3 |
| GO_PROTEIN_{—} | PRKN, RNF6, UBE2D4, UBE2S, UBE2T |
| K27_LINKED_{—} | |
| UBIQUITINATION | |
| GO_PROTEIN_{—} | ITCH, PRKN, RNF186, UBE2D4, UBE2S, UBE2T |
| K29_LINKED_{—} | |
| UBIQUITINATION | |
| GO_REGULATION_{—} | AIFM1, APAF1, BAX, CIDEA, DFFA, ENDOG, HSF1, NMNAT1, PAM16, ST20 |
| OF_APOPTOTIC_{—} | |
| DNA_FRAGMENTATION | |
| GO_REGULATION_{—} | CDCA5, HDAC8, NIPBL, RB1, WAPL |
| OF_COHESIN_{—} | |
| LOADING | |
| GO_REGULATION_{—} | CDCA5, HDAC8, NIPBL, RB1, SETD2, WAPL |
| OF_PROTEIN_{—} | |
| LOCALIZATION_{—} | |
| TO_CHROMATIN | |
| GO_RESPONSE_{—} | CCNB1, CYP1A1, DAXX, DDC, FECH, HNRNPA1, HSF1, KIF1B, MAPK13, NEFL, SCN1B, |
| TO_INSECTICIDE | SCN2B, STAR, TH, ZC3H12A |
| GO_RNA_{—} | BAZ2A, PIH1D1, RRN3, SMARCA4, SMARCB1, TAF1B, TAF1C, UBTF |
| POLYMERASE_I_{—} | |
| REGULATORY_{—} | |
| REGION_DNA_{—} | |
| BINDING | |
| GO_SUGAR_{—} | ADCY8, AGER, MLXIPL, PIH1D1, SMARCA4, SMARCB1 |
| MEDIATED_{—} | |
| SIGNALING_{—} | |
| PATHWAY | |
| PID_AURORA_A_{—} | AJUBA, AKT1, ARHGEF7, AURKA, AURKAIP1, AURKB, BIRC5, BRCA1, CDC25B, CENPA, |
| PATHWAY | CKAP5, CPEB1, DLGAP5, FZR1, GADD45A, GIT1, GSK3B, MDM2, NDEL1, NFKBIA, OAZ1, |
| | PAK1, PPP2R5D, PRKACA, RAN, RASA1, TACC1, TACC3, TDRD7, TP53, TPX2 |
| PID_AURORA_B_{—} | AC027237.1, AURKA, AURKB, AURKC, BIRC5, BUB1, CBX5, CDCA8, CENPA, CUL3, DES, EVI5, |
| PATHWAY | INCENP, KIF20A, KIF2C, KLHL13, KLHL9, MYLK, NCAPD2, NCAPG, NCAPH, NCL, NDC80, |
| | NPM1, NSUN2, PEBP1, PPP1CC, PPP2R5D, PSMA3, RACGAP1, RASA1, RHOA, SEPT1, SGO1, |
| | SMC2, SMC4, STMN1, TACC1, VIM |
| REACTOME_{—} | CCNB1, CCNB2, CDK1, NEK6, NEK7, NEK9, PLK1 |
| ACTIVATION_{—} | |
| OF_NIMA_{—} | |
| KINASES_{—} | |
| NEK9_NEK6_{—} | |
| NEK7 | |
| REACTOME_{—} | ACTR1A, AKAP9, ALMS1, AURKA, CCP110, CDK1, CDK5RAP2, CENPJ, CEP131, CEP135, |
| AURKA_{—} | CEP152, CEP164, CEP192, CEP250, CEP290, CEP41, CEP57, CEP63, CEP70, CEP72, CEP76, |
| ACTIVATION_{—} | CEP78, CETN2, CKAP5, CLASP1, CNTRL, CSNK1D, CSNK1E, DCTN1, DCTN2, DCTN3, |
| BY_TPX2 | DYNC1H1, DYNC1I2, DYNLL1, FGFR1OP, HAUS1, HAUS2, HAUS3, HAUS4, HAUS5, HAUS6, |
| | HAUS7, HAUS8, HMMR, HSP90AA1, MAPRE1, NDE1, NEDD1, NEK2, NINL, ODF2, OFD1, |
| | PAFAH1B1, PCM1, PCNT, PLK1, PLK4, PPP2R1A, PRKACA, PRKAR2B, SDCCAG8, SFI1, |
| | SSNA1, TPX2, TUBA1A, TUBA4A, TUBB, TUBB4A, TUBB4B, TUBG1, YWHAE, YWHAG |
| REACTOME_{—} | CCNA1, CCNA2, CCNB1, CCNB2, CCNH, CDC25A, CDC25B, CDC25C, CDK1, CDK2, CDK7, |
| CYCLIN_A_B1_B2_{—} | FOXM1, LCMT1, MNAT1, PKMYT1, PLK1, PPME1, PPP2CA, PPP2CB, PPP2R1A, PPP2R1B, |
| ASSOCIATED_{—} | PPP2R2A, PPP2R3B, WEE1, XPO1 |
| EVENTS_{—} | |
| DURING_G2_M_{—} | |
| TRANSITION | |
| REACTOME_{—} | CHUK, IKBKB, IKBKG, NFKB1, NFKB2, NFKBIA, RELA |
| IKBA_VARIANT_{—} | |
| LEADS_TO_{—} | |
| EDA_ID | |
| REACTOME_{—} | ACTR1A, AHCTF1, AKAP9, ALMS1, AURKB, B9D2, BIRC5, BUB1, BUB1B, BUB3, CCNB1, |
| MITOTIC_{—} | CCNB2, CCP110, CDC20, CDCA5, CDCA8, CDK1, CDK5RAP2, CENPA, CENPC, CENPE, CENPF, |
| PROMETAPHASE | CENPH, CENPI, CENPJ, CENPK, CENPL, CENPM, CENPN, CENPO, CENPP, CENPQ, CENPS, |
| | CENPT, CENPU, CEP131, CEP135, CEP152, CEP164, CEP192, CEP250, CEP290, CEP41, |
| | CEP57, CEP63, CEP70, CEP72, CEP76, CEP78, CETN2, CKAP5, CLASP1, CLASP2, CLIP1, |
| | CNTRL, CSNK1D, CSNK1E, CSNK2A1, CSNK2A2, CSNK2B, DCTN1, DCTN2, DCTN3, DSN1, |
| | DYNC1H1, DYNC1I1, DYNC1I2, DYNC1LI1, DYNC1LI2, DYNLL1, DYNLL2, ERCC6L, FGFR1OP, |
| | HAUS1, HAUS2, HAUS3, HAUS4, HAUS5, HAUS6, HAUS7, HAUS8, HDAC8, HSP90AA1, |
| | INCENP, ITGB3BP, KIF18A, KIF2A, KIF2B, KIF2C, KNL1, KNTC1, MAD1L1, MAD2L1, MAPRE1, |
| | MIS12, MZT1, MZT2A, MZT2B, NCAPD2, NCAPG, NCAPH, NDC80, NDE1, NDEL1, NEDD1, |
| | NEK2, NINL, NME7, NSL1, NUDC, NUF2, NUMA1, NUP107, NUP133, NUP160, NUP37, |
| | NUP43, NUP85, NUP98, ODF2, OFD1, PAFAH1B1, PCM1, PCNT, PDS5A, PDS5B, PLK1, PLK4, |
| | PMF1, PPP1CC, PPP2CA, PPP2CB, PPP2R1A, PPP2R1B, PPP2R5A, PPP2R5B, PPP2R5C, |
| | PPP2R5D, PPP2R5E, PRKACA, PRKAR2B, RAD21, RANBP2, RANGAP1, RCC2, RPS27, |
| | SDCCAG8, SEC13, SEH1L, SFI1, SGO1, SGO2, SKA1, SKA2, SMC1A, SMC2, SMC3, SMC4, |
| | SPC24, SPC25, SPDL1, SSNA1, STAG1, STAG2, TAOK1, TUBA1A, TUBA1B, TUBA1C, TUBA3C, |
| | TUBA3D, TUBA3E, TUBA4A, TUBA4B, TUBA8, TUBAL3, TUBB, TUBB1, TUBB2A, TUBB2B, |
| | TUBB3, TUBB4A, TUBB4B, TUBB6, TUBB8, TUBG1, TUBG2, TUBGCP2, TUBGCP3, TUBGCP4, |
| | TUBGCP5, TUBGCP6, WAPL, XPO1, YWHAE, YWHAG, ZW10, ZWILCH, ZWINT |
| REACTOME_{—} | AHCTF1, ANAPC1, ANAPC10, ANAPC11, ANAPC15, ANAPC16, ANAPC2, ANAPC4, ANAPC5, |
| MITOTIC_{—} | ANAPC7, AURKB, B9D2, BIRC5, BUB1, BUB1B, BUB3, CDC16, CDC20, CDC23, CDC26, |
| SPINDLE_{—} | CDC27, CDCA8, CENPA, CENPC, CENPE, CENPF, CENPH, CENPI, CENPK, CENPL, CENPM, |
| CHECKPOINT | CENPN, CENPO, CENPP, CENPQ, CENPS, CENPT, CENPU, CKAP5, CLASP1, CLASP2, CLIP1, |
| | DSN1, DYNC1H1, DYNC1I1, DYNC1I2, DYNC1LI1, DYNC1LI2, DYNLL1, DYNLL2, ERCC6L, |
| | INCENP, ITGB3BP, KIF18A, KIF2A, KIF2B, KIF2C, KNL1, KNTC1, MAD1L1, MAD2L1, MAPRE1, |
| | MIS12, NDC80, NDE1, NDEL1, NSL1, NUDC, NUF2, NUP107, NUP133, NUP160, NUP37, |
| | NUP43, NUP85, NUP98, PAFAH1B1, PLK1, PMF1, PPP1CC, PPP2CA, PPP2CB, PPP2R1A, |
| | PPP2R1B, PPP2R5A, PPP2R5B, PPP2R5C, PPP2R5D, PPP2R5E, RANBP2, RANGAP1, RCC2, |
| | RPS27, SEC13, SEH1L, SGO1, SGO2, SKA1, SKA2, SPC24, SPC25, SPDL1, TAOK1, UBE2C, |
| | UBE2D1, UBE2E1, XPO1, ZW10, ZWILCH, ZWINT |
| REACTOME_{—} | AHCTF1, AURKB, B9D2, BIRC5, BUB1, BUB1B, BUB3, CCNB1, CCNB2, CDC20, CDCA5, |
| RESOLUTION_{—} | CDCA8, CDK1, CENPA, CENPC, CENPE, CENPF, CENPH, CENPI, CENPK, CENPL, CENPM, |
| OF_SISTER_{—} | CENPN, CENPO, CENPP, CENPQ, CENPS, CENPT, CENPU, CKAP5, CLASP1, CLASP2, CLIP1, |
| CHROMATID_{—} | DSN1, DYNC1H1, DYNC1I1, DYNC1I2, DYNC1LI1, DYNC1LI2, DYNLL1, DYNLL2, ERCC6L, |
| COHESION | HDAC8, INCENP, ITGB3BP, KIF18A, KIF2A, KIF2B, KIF2C, KNL1, KNTC1, MAD1L1, MAD2L1, |
| | MAPRE1, MIS12, NDC80, NDE1, NDEL1, NSL1, NUDC, NUF2, NUP107, NUP133, NUP160, |
| | NUP37, NUP43, NUP85, NUP98, PAFAH1B1, PDS5A, PDS5B, PLK1, PMF1, PPP1CC, PPP2CA, |
| | PPP2CB, PPP2R1A, PPP2R1B, PPP2R5A, PPP2R5B, PPP2R5C, PPP2R5D, PPP2R5E, RAD21, |
| | RANBP2, RANGAP1, RCC2, RPS27, SEC13, SEH1L, SGO1, SGO2, SKA1, SKA2, SMC1A, SMC3, |
| | SPC24, SPC25, SPDL1, STAG1, STAG2, TAOK1, TUBA1A, TUBA1B, TUBA1C, TUBA3C, |
| | TUBA3D, TUBA3E, TUBA4A, TUBA4B, TUBA8, TUBAL3, TUBB1, TUBB2A, TUBB2B, TUBB3, |
| | TUBB4A, TUBB4B, TUBB6, TUBB8, WAPL, XPO1, ZW10, ZWILCH, ZWINT |
| REACTOME_{—} | ACTB, ACTG1, AHCTF1, AURKB, B9D2, BIRC5, BUB1, BUB1B, BUB3, CDC20, CDC42, CDCA8, |
| RHO_GTPASES_{—} | CENPA, CENPC, CENPE, CENPF, CENPH, CENPI, CENPK, CENPL, CENPM, CENPN, CENPO, |
| ACTIVATE_{—} | CENPP, CENPQ, CENPS, CENPT, CENPU, CKAP5, CLASP1, CLASP2, CLIP1, DAAM1, DIAPH1, |
| FORMINS | DIAPH2, DIAPH3, DSN1, DVL1, DVL2, DVL3, DYNC1H1, DYNC1I1, DYNC1I2, DYNC1LI1, |
| | DYNC1LI2, DYNLL1, DYNLL2, ERCC6L, EVL, FMNL1, FMNL2, FMNL3, INCENP, ITGB1, |
| | ITGB3BP, KIF18A, KIF2A, KIF2B, KIF2C, KNL1, KNTC1, MAD1L1, MAD2L1, MAPRE1, MIS12, |
| | MRTFA, NDC80, NDE1, NDEL1, NSL1, NUDC, NUF2, NUP107, NUP133, NUP160, NUP37, |
| | NUP43, NUP85, NUP98, PAFAH1B1, PFN1, PFN2, PLK1, PMF1, PPP1CC, PPP2CA, PPP2CB, |
| | PPP2R1A, PPP2R1B, PPP2R5A, PPP2R5B, PPP2R5C, PPP2R5D, PPP2R5E, RAC1, RANBP2, |
| | RANGAP1, RCC2, RHOA, RHOB, RHOC, RHOD, RPS27, SCAI, SEC13, SEH1L, SGO1, SGO2, |
| | SKA1, SKA2, SPC24, SPC25, SPDL1, SRC, SRF, SRGAP2, TAOK1, TUBA1A, TUBA1B, TUBA1C, |
| | TUBA3C, TUBA3D, TUBA3E, TUBA4A, TUBA4B, TUBA8, TUBAL3, TUBB1, TUBB2A, TUBB2B, |
| | TUBB3, TUBB4A, TUBB4B, TUBB6, TUBB8, XPO1, ZW10, ZWILCH, ZWINT |

One of skill in the art will be able to determine a suitable number of genes or gene sets or subsets thereof to be evaluated. In some embodiments, one or more sets of genes are analyzed (including all the genes in each set). A gene set can contain two or more (e.g., 2, 3, 4, 5, 6, 7, 8, 9, 10, 20, 50, 100 or more) genes that share a similar function, phenotype, or property (e.g., E2F target genes). For example, in some embodiments, all 51 gene sets listed in Table 2 are evaluated (e.g., via RNA-Seq analysis of the transcriptome). Alternatively, a subset of the gene sets listed in Table 2 (e.g., 1-10 gene sets) are evaluated. In some embodiments, one or more (e.g., all 73) gene sets listed in Table 8A are evaluated (e.g., via RNA-Seq analysis of the transcriptome). In some embodiments, one or more genes listed in Table 8B are evaluated. In some embodiments, some gene sets and/or subsets of genes contained within the disclosed gene sets are evaluated.

In other embodiments, one or more individual genes are analyzed, such as genes selected from Table 1, 2, 5A, 6A, 7A, 8B and/or 8C. For example, the one or more genes are not necessarily part of the same gene set. In some embodiments, 1-60, or any subset thereof, of any of the genes can be analyzed. In a preferred embodiment, from about 5 to about 40 genes, inclusive, selected from Table 1 and/or Table 2 are analyzed.

In some embodiments, one or more genes selected from Tables 10A, 11A, 12A, and/or 13C are associated with sensitivity to more than additive effects of the combination of an androgen antagonist or antiandrogen and one or more Plk inhibitors. Thus, the expression of one or more genes selected from Tables 10A, 11A, 12A, and/or 13C can be evaluated in identifying and/or selecting patients to be treated with the disclosed combination therapies.

In some embodiments, patients to be treated with the disclosed combination therapies show increased expression of one or more genes selected from KNL1, PRC1, HJURP, PSRC1, ASPM, CCNB2, CKAP2L, RACGAP1, DLGAP5, TROAP, KPNA2, and KIF14. In some embodiments, patients to be treated with the disclosed combination therapies show decreased expression of one or more genes selected from CLTC, XPO1, and HMGB3. In some embodiments, patients to be treated with the disclosed combination therapies show increased expression of one or more genes selected from KNL1, PRC1, HJURP, PSRC1, ASPM, CCNB2, CKAP2L, RACGAP1, DLGAP5, TROAP, KPNA2, and KIF14; and decreased expression of one or more genes selected from CLTC, XPO1, and HMGB3. In some embodiments, the increased or decreased expression is relative to the expression before exposure to a drug, such as an antiandrogen or androgen antagonist, or relative to patient(s) not treated with a drug, such as an antiandrogen or androgen antagonist. In other embodiments, the increased or decreased expression is relative to the distribution of expression observed in a population of patients.

Additionally, or alternatively to using the genes or gene sets as described above, cytological features (e.g., defects) can be evaluated to identify patients to be treated with the disclosed combination therapies. For example, cells from patient-derived samples can be examined to determine any evidence of any mitotic defect in response to a drug, such as an antiandrogen or androgen antagonist. Exemplary defects include improper spindle orientation, increased frequency of division not parallel to the growth surface, and mitotic arrest. In some embodiments, occurrence of such and/or other defects is correlated with susceptibility to the greater than additive effect of the combination therapy. Thus, patients whose sample(s) exhibit such cytological defects can be identified as likely to respond to the combination therapy.

2. Samples

The methods involving analyzing gene expression in one or more samples from a subject. The subject can be one with a disease or disorder in need of treatment, such as a cancer patient. The sample can contain cells that are characteristic of or otherwise affected by the disease or disorder. The sample can include a single cell, or preferably includes multiple cells. The sample can be tissue. Thus, in preferred embodiments, the biological sample is obtained from a tissue or organ that will exhibit symptoms or is otherwise associated with the disease or disorder to be treated.

For example, if the subject has cancer, the cells of the sample are typically cancer cells. In preferred embodiments, the sample includes cancer cells obtained from a tumor. In some embodiments, the sample includes cancer cells that are not obtained from a tumor. For example, in some embodiments, the cancer cells are circulating cancer cells or circulating tumor cells. The sample can include other components or cells that are not cancer cells. For example, the sample can include non-cancerous cells, tissue, etc. In preferred embodiments, the sample includes cancer cells isolated or separated away from normal tissue. In some embodiments, the sample is obtained from a cancerous tissue or organ. The sample can include cultured cells (e.g., primary cultures, explants, and transformed cells).

A sample can be fresh, frozen or fixed cells or tissue. In some embodiments, a sample includes formalin-fixed paraffin-embedded (FFPE) tissue, fresh tissue or fresh frozen (FF) tissue. A sample can include cultured cells, including primary or immortalized cell lines derived from a subject sample. A sample can also refer to an extract from a sample from a subject. For example, a sample can contain DNA, RNA or protein extracted from a tissue or a bodily fluid.

In some embodiments, the sample can be a biological fluid sample taken from a subject. Examples of biological samples include urine, blood, serum, plasma, saliva, cerebrospinal fluid, lymph, synovial fluid, sputum, broncheoalveolar lavage fluid, cyst fluid, pleural and peritoneal fluid, pericardial fluid, interstitial fluid, etc. In preferred embodiments, a fluid sample can be whole blood, serum or plasma. Serum is the component of whole blood that is neither a blood cell (serum does not contain white or red blood cells) nor a clotting factor. It is the blood plasma with the fibrinogens removed. Accordingly, serum includes all proteins not used in blood clotting (coagulation) and all the electrolytes, antibodies, antigens, hormones, and any exogenous substances (e.g., drugs and microorganisms). The sample can be diluted with a suitable diluent before the sample is analyzed.

In some embodiments, the sample contains one or more vesicles. Vesicles encompass membrane-bound compartments shed from cells. In some embodiments, vesicles are spherical structures with a lipid bilayer similar to cell membranes which surrounds an inner compartment which can contain soluble components. Vesicles include shed membrane bound particles, or “microparticles,” that are derived from either the plasma membrane or an internal membrane. Vesicles can be released into the extracellular environment from cells. Cells releasing vesicles include cells that originate from, or are derived from, the ectoderm, endoderm, or mesoderm. The cells can be tumor cells. A vesicle can reflect any changes in the source cell, and thereby reflect changes in the originating cells, e.g., cells having various genetic mutations or gene expression patterns. In one mechanism, a vesicle is generated intracellularly when a segment of the cell membrane spontaneously invaginates and is ultimately exocytosed (see for example, Keller et al., Immunol. Lett. 107 (2): 102-8 (2006)). Vesicles also include cell-derived structures bounded by a lipid bilayer membrane arising from both herniated evagination (blebbing) separation and sealing of portions of the plasma membrane, or from the export of any intracellular membrane-bounded vesicular structure containing various membrane-associated proteins of tumor origin, including surface-bound molecules derived from the host circulation that bind selectively to the tumor-derived proteins together with molecules contained in the vesicle lumen, including, but not limited to, tumor-derived microRNAs or intracellular proteins. A vesicle shed into circulation or bodily fluids from tumor cells may be referred to as a “circulating tumor-derived vesicle.” When such vesicle is an exosome, it may be referred to as a circulating-tumor derived exosome (CTE).

Suitable vesicles include circulating microvesicles (cMVs), microvesicles, exosomes, nanovesicle, dexosomes, intralumenal vesicle, membrane fragment, intralumenal endosomal vesicle, endosomal-like vesicle, phospholipid vesicle, liposomal vesicle, and oncosomes.

In some embodiments, vesicles are directly assayed from a biological sample without prior isolation, purification, or concentration from the biological sample. Alternatively, the vesicle in the sample may be isolated, captured, purified, or concentrated from a sample prior to analysis. Vesicle isolation can be performed using various techniques as described herein or known in the art, including size exclusion chromatography, density gradient centrifugation, differential centrifugation, nanomembrane ultrafiltration, immunoabsorbent capture, affinity purification, affinity capture, immunoassay, immunoprecipitation, microfluidic separation, flow cytometry or combinations thereof.

In some embodiments, the sample can be a formalin fixed paraffin embedded (FFPE) sample. The FFPE sample can be one or more of fixed tissue, unstained slides, bone marrow core or clot, core needle biopsy, malignant fluids and fine needle aspirate (FNA). In some embodiments, the fixed tissue contains a tumor containing formalin fixed paraffin embedded (FFPE) block from a surgery or biopsy.

In preferred embodiments, the sample is a tissue biopsy, tumor biopsy, blood sample, or cells (e.g., cancer cells such as circulating tumor cells (CTCs)) obtained from the subject.

A sample can be obtained from the subject using a variety of methods that are known in the art. In some embodiments, the sample is a tissue biopsy, for example a punch biopsy. The sample should be handled in accordance with the method of detection that will be employed.

In some embodiments, a biological sample that is of tissue or cellular origin can be solubilized in a lysis buffer optionally containing a chaotropic agent, detergent, reducing agent, buffer, and/or salts. The conditions for handling biological samples that are analyzed for mRNA level may be different than the conditions for handling biological samples that are analyzed for protein level, and such conditions are known in the art. If the sample is a blood sample that includes clotting factors (e.g., a whole blood sample), the preparation may include an anti-coagulant.

3. Analyzing Expression

The detection of mRNA, polypeptides and proteins in a sample obtained from a subject is made possible by a number of conventional methods that are known in the art. The methods can be cell-based or cell-free assays.

For example, mRNA levels can be determined using assays, including, but not limited to, RT-PCR, reverse transcription real-time PCR (RT-qPCR), ddPCR, transcriptome analysis using next-generation sequencing (e.g., RNA-sequencing), single-cell RNA-Sequencing, miRNA-Seq, microarrays, NanoString, Luminex assays array analysis, digital PCR, in situ hybridization, and northern analysis. In a preferred embodiment, the method includes detecting the level of one or more RNA transcripts in RNA isolated from cells of the subject. In some embodiments, a probe for detecting a gene or biomarker is designed to hybridize with the nucleic acid sequence encoding the biomarker, or a complement thereof.

Protein expression can be detected using routine methods, such as immunodetection methods, flow cytometry, immunofluorescence microscopy, mass spectroscopy, or high-performance liquid chromatography (HPLC). In a preferred embodiment, the method includes detecting the level of biomarker protein or polypeptide, or a combination thereof in protein isolated from cells of the subject.

Some methods include an immunoassay whereby polypeptides of the genes or biomarker are evaluated or detected by their interaction with a biomarker-specific antibody. The biomarker can be detected in either a qualitative or quantitative manner. Exemplary immunoassays that can be used for the detection of biomarker polypeptides and proteins include, but are not limited to, radioimmunoassays, ELISAs, immunoprecipitation assays, Western blot, fluorescent immunoassays, and immunohistochemistry, flow cytometry, protein arrays, multiplexed bead arrays, magnetic capture, in vivo imaging, fluorescence resonance energy transfer (FRET), and fluorescence recovery/localization after photobleaching (FRAP/FLAP).

Some immunoassays, for example ELISAs, can require two different biomarker specific antibodies or ligands (e.g., a capture ligand or antibody, and a detection ligand or antibody). In certain embodiments, the protein biomarker is captured with a ligand or antibody on a surface and the protein biomarker is labeled with an enzyme. In one example, a detection antibody conjugated to biotin or streptavidin can be used to create a biotin-streptavidin linkage to an enzyme that contains biotin or streptavidin. A signal is generated by the conversion of the enzyme substrate into a colored molecule and the intensity of the color of the solution is quantified by measuring the absorbance with a light sensor. Assays may utilize chromogenic reporters and substrates that produce an observable color change to indicate the presence of the protein biomarker. Fluorogenic, electrochemiluminescent, and real-time PCR reporters are also contemplated to create quantifiable signals.

Some assays optionally including fixing one or more antibodies to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe, substrate or a PROTEINCHIP® array.

Flow cytometry is a laser-based technique that may be employed in counting, sorting, and detecting protein biomarkers by suspending particles in a stream of fluid and passing them by an electronic detection apparatus. A flow cytometer has the ability to discriminate different particles on the basis of color. Differential dyeing of particles with different dyes, emitting in two or more different wavelengths allows the particle to be distinguished. Multiplexed analysis, such as FLOWMETRIX™ is discussed in Fulton, et al., Clinical Chemistry, 43(9):1749-1756 (1997) and can allow one to perform multiple discrete assays in a single tube with the same sample at the same time.

In some specific embodiments, the biomarker level(s) are measured using LUMINEX XMAP® technology. LUMINEX XMAP® is frequently compared to the traditional ELISA technique, which is limited by its ability to measure only a single analyte. The differences between ELISA and LUMINEX XMAP® technology center mainly on the capture antibody support. Unlike with traditional ELISA, LUMINEX XMAP® capture antibodies are covalently attached to a bead surface, effectively allowing for a greater surface area as well as a matrix or free solution/liquid environment to react with the analytes. The suspended beads allow for assay flexibility in a singleplex or multiplex format.

Commercially available formats that include Luminex xMAP® technology includes, for example, BIO-PLEX® multiplex immunoassay system which permits the multiplexing of up to 100 different assays within a single sample. This technique involves 100 distinctly colored bead sets created by the use of two fluorescent dyes at distinct ratios. These beads can be further conjugated with a reagent specific to a particular bioassay. The reagents may include antigens, antibodies, oligonucleotides, enzyme substrates, or receptors. The technology enables multiplex immunoassays in which one antibody to a specific analyte is attached to a set of beads with the same color, and the second antibody to the analyte is attached to a fluorescent reporter dye label. The use of different colored beads enables the simultaneous multiplex detection of many other analytes in the same sample. A dual detection flow cytometer can be used to sort out the different assays by bead colors in one channel and determine the analyte concentration by measuring the reporter dye fluorescence in another channel.

In some specific embodiments, the biomarker(s) levels are measured using Quanterix's SIMOA™ technology. SIMOA™ technology (named for single molecule array) is based upon the isolation of individual immunocomplexes on paramagnetic beads using standard ELISA reagents. The main difference between Simoa and conventional immunoassays lies in the ability to trap single molecules in femtoliter-sized wells, allowing for a “digital” readout of each individual bead to determine if it is bound to the target analyte or not. The digital nature of the technique allows an average of 1000× sensitivity increase over conventional assays with CVs <10%. Commercially available SIMOA™ technology platforms offer multiplexing options up to a 10-plex on a variety of analyte panels, and assays can be automated.

Multiplexing experiments can generate large amounts of data. Therefore, in some embodiments, a computer system is utilized to automate and control data collection settings, organization, and interpretation.

Besides the genes or other biomarkers, measurement may also be made of other indirect genomic/genetic markers that are associated with sensitivity to more than additive effects of the combination therapies. This can include, for example, somatic mutations, genomic amplifications or other genomic copy number variations, mitotic spindle assembly, centriole location and/or number, chromosomal alignments or pairing, chromatin structure, other physical cell cycle related structures or compartments, etc. These can be assessed using known cytological assays such as microscopy, smears, metaphase spreads, fine needle aspiration (FNA), etc.

4. Controls

The methods including analyzing one or more genes or other biomarkers typically includes comparing to a control. For example, the expression level of a biomarker detected in a sample obtained from the subject can be compared to a control. Suitable controls will be known to one of skill in the art. Controls can include, for example, standards obtained from healthy subjects, such as subjects without the disease or disorder, or non-diseased tissue from the same subject. A control can be a single or pooled or averaged values of like individuals using the same assay. Reference indices can be established by using subjects that have been diagnosed with the disease or disorder with different known disease severities or prognoses. The control biological sample(s) can be assayed using the same methods as the test sample.

In some embodiments, the control can include similar cells to the sample but without the disease (e.g., expression profiles obtained from samples from healthy individuals). A control can be a previously determined level that is indicative of a cancer cell's or tumor's sensitivity to more than additive effects of the combination therapies. The control can be derived from the same patient, e.g., a normal adjacent portion of the same organ as the diseased cells, the control can be derived from healthy tissues from other patients, or previously determined thresholds that are indicative of a disease responding or not-responding to a particular drug target. In some embodiments, a control can be a control found in the same sample, e.g. a housekeeping gene or a product thereof (e.g., mRNA or protein). Multiple controls or types of controls can be used.

In certain embodiments, a control is the expression of the biomarker in the same sample before or after a certain manipulation (e.g., treatment with a drug). For example, one or more genes from a sample can be determined to be expressed or upregulated (or decreased/downregulated) relative to the expression before exposure to a drug, such as an antiandrogen or androgen antagonist. The exposure to a drug can be in vivo (e.g., a patient is administered the drug) or ex vivo (e.g., tissue or cells cultured with or otherwise exposed to the drug). For example, in some embodiments, cancer cells isolated from a patient can be split into two groups with one group of cells remaining untreated or treated with a vehicle control (e.g., DMSO) and a second group of cells being treated with a drug (e.g., antiandrogen or androgen antagonist). The expression levels can be compared across these two conditions. The patient may be treatment naive before isolation of the sample (e.g., the patient has not previously received or is not currently receiving an antiandrogen or androgen antagonist). In other embodiments, the expression of one or more genes in a sample from a patient isolated after the patient is treated with a drug (e.g., antiandrogen or androgen antagonist) is compared to the expression of the one or more genes in a sample from a patient that was isolated before the patient was treated with the drug (e.g., antiandrogen or androgen antagonist). In other embodiments, the expression of one or more genes in a sample from a patient isolated after the patient is treated with a drug (e.g., antiandrogen or androgen antagonist) is compared to the expression of the one or more genes in a sample from patient(s) that have not been treated with the drug (e.g., antiandrogen or androgen antagonist). While the controls described above are discussed in the context of gene expression, it is to be understood that the same controls are applicable in the context of other biomarkers that are associated with sensitivity to more than additive effects of the combination therapies. This includes somatic mutations, genomic amplifications or other genomic copy number variations, mitotic spindle assembly, centriole location and/or number, chromosomal alignments or pairing, chromatin structure, other physical cell cycle related structures or compartments, etc.

The expression levels of the one or more genes can differ from the control by any magnitude and direction (e.g., unchanged, upregulated, or downregulated). A gene or gene product can be considered up or down-regulated if the differential expression meets a statistical threshold, a fold-change threshold, or both. For example, upregulation or downregulation may encompass, but do not necessarily denote any level of statistical significance. For example, a gene can be considered as upregulated when the fold-change in expression compared to control level is increased by at least 1.1, 1.2, 1.3, 1.4, 1.5, 1.6, 1.7, 1.8, 1.9, 2.0, 2.2, 2.5, 2.7, 3.0, 4, 5, 6, 7, 8, 9 or 10-fold in the sample versus the control. As another example, a gene can be considered as downregulated when the fold-change in expression compared to control level is reduced by at least 1.1, 1.2, 1.3, 1.4, 1.5, 1.6, 1.7, 1.8, 1.9, 2.0, 2.2, 2.5, 2.7, 3.0, 4, 5, 6, 7, 8, 9 or 10-fold in the sample versus the control.

In some embodiments, the criteria for identifying differential expression can include both a p-value and a fold change (up or down). One of skill will understand that such statistical and threshold measures can be adapted to determine differential expression by any molecular profiling technique.

Assessment can be performed using a statistical test to determine statistical significance of any differential expression observed. In some embodiments, statistical significance is determined using a parametric or nonparametric statistical test. Suitable tests include, for example, a fractional factorial design, analysis of variance (ANOVA), a t-test, least squares, a Pearson correlation, simple linear regression, nonlinear regression, multiple linear regression, multiple nonlinear regression, Wilcoxon signed-rank test, a Mann-Whitney test, a Kruskal-Wallis test, a Friedman test, a Spearman ranked order correlation coefficient, a Kendall Tau analysis, and a nonparametric regression test. In some embodiments, statistical significance is determined at a p-value of less than about 0.05, 0.01, 0.005, 0.001, 0.0005, or 0.0001.

B. Methods of Treatment

The methods of treatment typically include administering a subject an effective amount of the active agents to prevent, reduce, or treat one or more symptoms of a disease or disorder.

The combination therapies and treatment regimens typically include treatment of a disease or symptom thereof, or a method for achieving a desired physiological change, including administering to a subject an effective amount of an antiandrogen or androgen antagonist and a Plk inhibitor to treat the disease or symptom thereof, or to produce the physiological change. The active agents (e.g., antiandrogen or androgen antagonist) can be administered together, such as part of the same composition, or administered separately and independently at the same time or at different times (e.g., administration of the antiandrogen or androgen antagonist and the Plk inhibitor is separated by a finite period of time from each other). Therefore, the term “combination” or “combined” is used to refer to either concomitant, simultaneous, or sequential administration of the antiandrogen or androgen antagonist and the Plk inhibitor. The combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one agent is given first followed by the second).

The compositions and methods are useful for treating subjects having benign or malignant tumors by delaying or inhibiting the growth of a tumor in a subject, reducing the growth or size of the tumor, inhibiting or reducing metastasis of the tumor, inhibiting or reducing cancer cell proliferation and/or viability, and/or inhibiting or reducing symptoms associated with tumor development or growth.

Methods of treating cancer or one or more symptoms of cancer by administering the combination therapies are described. In particular embodiments, the method of treating cancer or one or more symptoms of cancer includes administering an antiandrogen or androgen antagonist in combination with a Plk inhibitor to a subject. When used in methods for treating cancer, the amount of antiandrogen or androgen antagonist present in a pharmaceutical dosage unit, or otherwise administered to a subject can be the amount effective to reduce the proliferation, viability, or a combination thereof of the cancer cells when administered in combination with a Plk inhibitor. Likewise, the amount of Plk inhibitor present in a pharmaceutical dosage unit, or otherwise administered to a subject can be the amount effective to reduce the proliferation, viability, or a combination thereof of the cancer cells when administered in combination with an antiandrogen or androgen antagonist. Therefore, in some embodiments the amount of the active agents is effective to reduce, slow or halt tumor progression, to reduce tumor burden, or a combination thereof. In some embodiment, the amount of the active agents is effective to alter a measureable biochemical or physiological marker. For example, if the cancer is prostate cancer, the amount of the active agents can be effective to reduce the level of prostate specific antigen (PSA) concentration in the blood compared to the PSA concentration prior to treatment.

In preferred embodiments, administration of the antiandrogen or androgen antagonist and the Plk inhibitor achieves a result greater than when the antiandrogen or androgen antagonist and the Plk inhibitor are administered alone or in isolation. For example, in some embodiments, the result achieved by the combination is partially or completely additive of the results achieved by the individual components alone. In the most preferred embodiments, the result achieved by the combination is more than additive of the results achieved by the individual components alone. In some embodiments, the effective amount of one or both agents used in combination is lower than the effective amount of each agent when administered separately. In some embodiments, the amount of one or both agents when used in the combination therapy is sub-therapeutic when used alone.

The effect of the combination therapy, or individual agents thereof can depend on the disease or condition to be treated or progression thereof. For example, an agent such as abiraterone can be used as a first or second line therapy for treatment of prostate cancer. However, over time, the cancer can develop a resistance to abiraterone. Subsequent treatment of the cancer with abiraterone in combination with a Plk inhibitor such as onvansertib can reduce cancer cell viability since abiraterone treatment can “sensitize” the cancer to Plk inhibition. Accordingly, in some embodiments, the effect of the combination on a cancer can be compared to the effect of the individual agents alone on the cancer.

The effect of the combination therapies can be hormone independent. The effect of the combination can be improved over the individual components alone. In some embodiments, although the cancer killing effect of the combination is similar to the individual components, the duration of efficacy of the treatment is longer because the cancer does not become resistant to the treatment. This allows the combination therapies to be administered in combination with or as an alternative to hormone therapy, a first line therapy, or a second line or subsequent therapy, and without the need of the cancer to first become resistant to the antiandrogen or androgen antagonist (e.g., abiraterone).

A treatment regimen of the combination therapy can include one or multiple administrations of antiandrogen or androgen antagonist. A treatment regimen of the combination therapy can include one or multiple administrations of Plk inhibitor. In certain embodiments, an antiandrogen or androgen antagonist can be administered simultaneously with a Plk inhibitor.

In some embodiments an antiandrogen or androgen antagonist and a Plk inhibitor are administered sequentially, for example, in two or more different pharmaceutical compositions. In certain embodiments, the antiandrogen or androgen antagonist is administered prior to the first administration of the Plk inhibitor. In other embodiments, the Plk inhibitor is administered prior to the first administration of the antiandrogen or androgen antagonist. For example, the antiandrogen or androgen antagonist and the Plk inhibitor can be administered to a subject on the same day. Alternatively, the antiandrogen or androgen antagonist and the Plk inhibitor are administered to the subject on different days.

The Plk inhibitor can be administered at least 1, 2, 3, 5, 10, 15, 20, 24 or 30 hours or days prior to or after administering the antiandrogen or androgen antagonist. Alternatively, the antiandrogen or androgen antagonist can be administered at least 1, 2, 3, 5, 10, 15, 20, 24 or 30 hours or days prior to or after administering the Plk inhibitor. In certain embodiments, additive or more than additive effects of the administration of antiandrogen or androgen antagonist in combination with one or more Plk inhibitors is evident after one day, two days, three days, four days, five days, six days, one week, or more than one week following administration.

Dosage regimens or cycles of the agents can be completely or partially overlapping, or can be sequential. For example, in some embodiments, all such administration(s) of the antiandrogen or androgen antagonist occur before or after administration of the Plk inhibitor. Alternatively, administration of one or more doses of the antiandrogen or androgen antagonist can be temporally staggered with the administration of Plk inhibitor to form a uniform or non-uniform course of treatment whereby one or more doses of antiandrogen or androgen antagonist are administered, followed by one or more doses of Plk inhibitor, followed by one or more doses of antiandrogen or androgen antagonist; or one or more doses of Plk inhibitor are administered, followed by one or more doses of antiandrogen or androgen antagonist, followed by one or more doses of Plk inhibitor; etc. The dosage regimens or cycles can vary in length (e.g., 14, 21, 28 days). The regimens can include administering one agent daily for a specific number of days in the cycle while the other agent is administered daily for the entire duration of the cycle. For example, in some embodiments, the antiandrogen or androgen antagonist is administered daily and the Plk inhibitor is administered on days 1 through 5 of a 14 or 21 day cycle. In some embodiments, the antiandrogen or androgen antagonist is administered daily and the Plk inhibitor is administered on days 1 through 14 of a 21 day cycle. In some embodiments, the antiandrogen or androgen antagonist is administered daily and the Plk1 inhibitor is administered on days 1 through 9 of a 14 day cycle. In some embodiments, the antiandrogen or androgen antagonist and the Plk inhibitor are both administered daily.

The dosing schedule and length of treatment can be determined by the researcher or clinician administering the therapy.

An effective amount of each of the agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days, or up to 20 days, or up to 25 days, all of which are specifically contemplated.

C. Patient Populations

In some embodiments, the cancer patients to be treated are identified as patients likely to respond to or experience efficacious treatment by a combination therapy including antiandrogens or androgen antagonists and Plk inhibitors.

In some embodiments, patients to be treated have cancers or other patient-derived sample that exhibit expression or upregulation of one or more genes or gene products involved in mitosis, meiosis, the mitotic spindle, mitotic spindle assembly and/or checkpoint, microtubule organization, the centromere, the kinetochore, G2M checkpoint, E2F target genes, or combinations thereof. In some embodiments, patients to be treated have cancers or other patient-derived sample that exhibit expression or upregulation of one or more genes or gene sets provided in Table 1, Table 2, or combinations thereof. In some embodiments, the expression or upregulation can be in response to treatment with an antiandrogen or androgen antagonist or relative to a sample that has not been treated with an antiandrogen or androgen antagonist.

In some embodiments, patients to be treated are identified as having a particular molecular subtype of cancer. For example, the patients can have cancer (e.g., prostate cancer) of the basal subtype. In some embodiments, basal indicates that the tumor cells have a gene signature indicative of a basal epithelial cell origin. In some preferred embodiments, patients having basal mCRPC are treated with a combination therapy including antiandrogens or androgen antagonists and Plk inhibitors.

When administered the combination therapy, patients having the above characteristics will experience a therapeutic effect (e.g., reduction in cancer cell proliferation, viability, metastasis, tumor burden, tumor progression, and combinations thereof) to a greater degree than if they were administered the same amount of antiandrogen or androgen antagonist alone or the same amount of Plk inhibitor alone. In preferred embodiments, the therapeutic effect is more than the additive reduction achieved by administering the individual agents.

D. Diseases to be Treated

The combination therapies are typically used to treat cancer. The cancer can be a hormone-sensitive or hormone-dependent cancer that has become hormone-insensitive (also referred to as castrate-resistant). Typically, hormone-sensitive cancers are initially dependent on a hormone for cancer growth. Altering the cancer's hormone supply through hormone therapy such as drugs that alter hormone production, anti-hormones, aromatase inhibitors, Luteinizing hormone-releasing hormone (LH-RH) agonists and antagonists, or surgery to remove hormone producing tissue or organs, can make the tumors shrink and even lead to cancer remission. However, the effects of hormone therapy can be limited. Hormone sensitive cancers often become hormone-insensitive, meaning the cancer is no longer responsive to hormone therapy, although in most cases the tumor is still driven by hormonal signaling. The combination therapies are particularly effective for treating hormone-sensitive cancers that have become hormone-insensitive.

In some embodiments, the combination therapies are used to treat subjects with a hormone-insensitive cancer that is also resistant to a first line therapy, a second line therapy, or a combination thereof. In some cases, the first line therapy, second line therapy, or a combination thereof includes the administration of one of the active agents of the combination therapy without co-administration of the other active agent. Therefore, in some embodiments administration of the combination therapy re-sensitizes the cancer cells to an active agent that was previously administered to the subject as a first or a second line therapy.

In some embodiments, the cancer is an androgen-sensitive cancer that has become androgen-insensitive (also referred to as castrate-resistant or androgen-independent cancer). Androgen-independent cancer is typically a cancer that has reacquired an ability to grow following temporary suppression of the cancer's ability to grow by inhibiting androgen production or function. In some embodiments, suppression of the cancer's ability to grow refers to suppression of tumor growth or another symptom of the cancer, for example, amelioration of ostealgia. This suppression can be measured using a biochemical assay, for example, measuring a decline in the prostate specific antigen (PSA) concentration in the blood, or by a morphometric analysis, for example by computerized tomography (CT), magnetic resonance imaging (MRI) or ultrasound.

A cancer that has reacquired an ability to grow can exhibit an increase in tumor growth, the emergence, reemergence, or aggravation of other symptoms such as ostealgia, new sites of metastasis, or a rise in blood PSA. A sustained rise in blood PSA concentration observed in the course of periodic tests can indicate that the cancer has reacquired the ability to grow.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas are derived from mesodermal connective tissues such as bone, fat, and cartilage. The leukemias and lymphomas are malignant tumors of hematopoietic ceils of the bone marrow. Leukemias proliferate as single cells, whereas lymphomas tend to grow as tumor masses. Malignant tumors may show up at numerous organs or tissues of the body to establish a cancer. The compositions and methods may be effective in treating any of the foregoing.

Cancers that can be treated with the provided compositions and methods include, but are not limited to, prostate cancer, breast cancer, ovarian cancer, colorectal cancer, pancreatic cancer (e.g., pancreatic adenocarcinoma or pancreatic exocrine cancer), head and neck cancer, acute myeloid leukemia, lymphoma, B cell lymphoma, T cell lymphoma, mycosis fungoides, Hodgkin's Disease, endometrial cancer, lung cancer (e.g., small cell lung cancer and non-small cell lung cancer), neuroblastoma, glioblastoma, skin cancer, liver cancer, melanoma, squamous cell carcinomas of the mouth, throat, larynx, and lung, colon cancer, cervical cancer, cervical carcinoma, epithelial cancer, renal cancer, genitourinary cancer, pulmonary cancer, esophageal carcinoma, large bowel cancer, hematopoietic cancers, testicular cancer, uterine cancer, and rectal cancer.

In some embodiments, the compositions are used to treat multiple cancer types concurrently. The compositions can be used to treat metastases or tumors at multiple locations. In some embodiments, the cancers to be treated are characterized as having one or more KRAS-mutations, EGFR mutations, RB1 mutations, or combinations thereof. Non-hormonal cancers can also be treated. In some embodiments, the cancer does not express or over express AR or ER. In some embodiments, the cancer is an androgen receptor-positive breast cancer. In some embodiments, the breast cancer lacks ER, PR, or HER2 expression, or a combination thereof (e.g., triple negative breast cancer). In some embodiments, the breast cancer is a hormone-insensitive cancer, is resistant to a HER2 directed therapy, or any combination thereof.

Gene expression profiles for cancer cells within a tumor or for cells within the tumor microenvironment can be determined in vitro or in vivo by any means known in the art. Genomic databases can also be used as a guide for the selection of pharmacologic vulnerabilities to genomic patterns. Such databases include the Cancer Cell Line Encyclopedia (CCLE), including gene expression profile information for human cancer cell lines (Stransky, et al., Nature 483, 603-307 (2012)).

### Types of Cancer Having Shown Sensitivity

In preferred embodiments, the cancer to be treated is prostate cancer. Prostate cancer is the most frequently diagnosed malignancy in men in Western countries. While localized prostate cancer can be effectively treated with surgery or radiation therapy, metastatic PCa still remains incurable. For locally advanced or widespread disease, suppressing the tumor growth by hormone ablation therapy represents the common first therapeutic option (Beltran, et al., European Urology, 60:279-290 (2011)). Although initial therapy can lead to long-term remission, development of hormone ablation resistance can eventually occur, a standing referred to as castration-resistant prostate cancer (CRPC). Therefore, in some embodiments, the subject has a CRPC. Unlike early prostate cancer, CRPC is an aggressive disease that progresses despite castrate levels of testosterone (≤50 ng/ml). In some embodiments, the cancer to be treated is metastatic CRPC. In some embodiments, the cancer to be treated is basal type prostate cancer.

Subjects with CRPC are typically administered a first line therapy. Abiraterone and enzalutamide are exemplary first line treatment options for patients with CRPC. Second-line treatments following first line treatment failure include Docetaxel, cabazitaxel and sipuleucel-T. The history of first and second line therapeutic options for subjects with CRPC are reviewed in Lorente, et. al., Lancet Oncol., 16(6): e279-e292 (2015), which is specifically incorporated by reference herein in its entirety. The combination therapies can be administered to subjects which have previously been administered a first line therapy for CRPC and/or a second line therapy for CRPC. In a particular embodiment, the combination therapy is administered to a subject when a first line therapy and/or a second line therapy have become ineffective to treat or prevent progression of the cancer.

In a particular embodiment, the combination therapy is administered to a subject that was previously administered an antiandrogen or androgen antagonist. In some embodiments, the agent can target androgen receptor activity directly, or indirectly, for example by inhibiting androgen synthesis. In a particular embodiment, the subject was previously administered an abiraterone-based therapy such as ZYTIGA® (abiraterone acetate). Abiraterone has been administered to subjects as a first line therapy and as a second line therapy, typically following chemotherapy, for treatment of CRPC. The data presented in the Examples illustrates that the combination therapies are effective to treat castration-resistant prostate cancer.

Other types of cancer for which efficacy has been demonstrated include ovarian cancer and pancreatic cancer.

### III. Compositions

Compositions for use in the treatment of the diseases are also provided. For example, compositions for combination therapies including an antiandrogen or androgen antagonist for use in a method of treating a subject with cancer are provided. The combination therapies include administration of an effective amount of at least two active agents, one being an antiandrogen or androgen antagonist and the other being a polo-like kinase inhibitor, to a subject in need thereof. These would typically be provided in separate dosage units, or in a kit, although in some cases could be co-formulated.

In some embodiments, a composition includes a Plk inhibitor for use in a method of treating a subject with cancer, wherein the subject is one whom a composition including an antiandrogen or androgen antagonist has previously been or is currently being administered and wherein the response achieved following the administration of the Plk inhibitor is greater than the response achieved by administering either the antiandrogen or androgen antagonist alone or the Plk inhibitor alone. In some embodiments, a composition includes an antiandrogen or androgen antagonist for use in a method of treating a subject with cancer, wherein the subject is one whom a composition including a Plk inhibitor has previously been or is concurrently being administered and wherein the response achieved following the administration of antiandrogen or androgen antagonist is greater than the response achieved by administering either the antiandrogen or androgen antagonist alone or the Plk inhibitor alone.

A. Therapeutic Agents

1. Antiandrogen or Androgen Antagonist

The combination therapies include an antiandrogen or androgen antagonist. The antiandrogen or androgen antagonist can reduce or inhibit the androgen synthesis pathway, or reduce or inhibit binding of endogenous ligands including, but not limited to, testosterone and dihydrotestosterone (DHT), to the androgen receptor, or a combination thereof. In preferred embodiments, the antiandrogen or androgen antagonist reduces or inhibits synthesis of testosterone or DHT. In some embodiments, the antiandrogen or androgen antagonist reduces or inhibits expression or activity of one or more enzymes or cofactors in the steroid synthetic pathway. In a preferred embodiment, the antiandrogen or androgen antagonist reduces expression or activity of 17 alpha-hydroxylase, C17, 20-lyase, 5 alpha-reductase, or a combination thereof.

In some embodiments, the antiandrogen or androgen antagonist reduces expression or activity of the androgen receptor, reduces or inhibits ligand binding to the androgen receptor, reduces or inhibits translocation of the receptor to the nucleus, reduces or inhibits the activity of the receptor in the nucleus, or a combination thereof. For example, in some embodiments, the antiandrogen or androgen antagonist can target the androgen receptor signaling pathway. However, suitable antiandrogens or androgen antagonists are not limited to agents which reduce expression or activity of the androgen receptor/androgen receptor signaling.

### Steroidal Antiandrogen or Androgen Antagonists

In some embodiments, the antiandrogen or androgen antagonist is a steroid or has a steroidal structure. “Steroid” or “steroidal structure” as used herein typically refers to molecules having the ABCD ring characteristic of steroids.

In one embodiment, the antagonist has the formula:

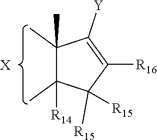

where X represents the A, B, and C rings of a steroid, R₁₄ is hydrogen, R₁₅ is hydrogen or C₁-C₆, preferably C₁-C₄ substituted or unsubstituted alkyl or alkoxy, hydroxy, or alkylcarbonyloxy having 2-6, preferably 2-5 carbons or R₁₄ and R₁₅ together represent a double bond, R₁₆ is hydrogen or C₁-C₆, preferably C₁-C₄ substituted or unsubstituted alkyl, and Y is a substituted or unsubstituted heterocycle or fused heterocycle. In some embodiments, Y is a substituted or unsubstituted fused heterocycle.

In a particular embodiment, the antagonist is TOK-001 (also referred to as galeterone) which has the following structure:

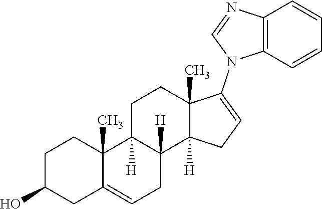

In one embodiment, the antagonist has the formula:

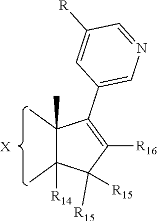

where X represents the A, B, and C rings of a steroid, R is a hydrogen or C₁-C₆, preferably C₁-C₄ substituted or unsubstituted alkyl, R₁₄ is hydrogen, R₁₅ is hydrogen or C₁-C₆, preferably C₁-C₄ substituted or unsubstituted alkyl or alkoxy, hydroxy, or alkylcarbonyloxy having 2-6, preferably 2-5 carbons or R₁₄ and R₁₅ together represent a double bond, and R₁₆ is hydrogen or C₁-C₆, preferably C₁-C₄ substituted or unsubstituted alkyl.

In the most preferred embodiments, the antiandrogen or androgen antagonist is abiraterone, or a prodrug, analog, derivative, or pharmaceutically acceptable salt thereof. The structure of abiraterone is shown below:

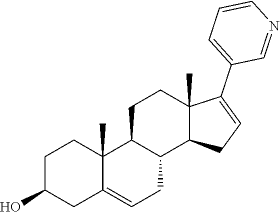 [SMILES: C[C@]12CC[C@H](O)CC1=CC[C@@H]1[C@@H]2CC[C@]2(C)C(c3cccnc3)=CC[C@@H]12]

Abiraterone, as well as prodrugs, analogs, derivatives, or pharmaceutically acceptable salts thereof are known in the art. See, for example, U.S. Pat. No. 5,604,213 which is specifically incorporated by reference herein in its entirety.

Abiraterone inhibits 17 α-hydroxylase/C17,20 lyase (CYP17A1), an enzyme which is expressed in testicular, adrenal, and prostatic tumor tissues. CYP17 catalyzes two sequential reactions: (a) the conversion of pregnenolone and progesterone to their 17-α-hydroxy derivatives by its 17 α-hydroxylase activity, and (b) the subsequent formation of dehydroepiandrosterone (DHEA) and androstenedione, respectively, by its C17,20 lyase activity. DHEA and androstenedione are androgens and precursors of testosterone, therefore, inhibition of CYP17 activity by abiraterone decreases circulating levels of testosterone. It is also believed that abiraterone and/or its metabolic derivatives act as direct androgen receptor antagonists (see e.g., Li, et. al., Nature. 523(7560) 347-351 (2015); Richards, et. al., Cancer Research. 72(9) 2176-2182 (2012)).

Suitable dosages of abiraterone when used as a first or second line therapy for treatment of cancer are also known in the art. For example, U.S. Pat. No. 5,604,213 teaches that a therapeutically effective dose can be in the range 0.001-0.04 mmole/kg body weight, preferably 0.001-0.01 mmole/kg, administered daily or twice daily during the course of treatment. In some embodiments, the dosage is 10-2,000 mg/patient per day, 100-1,500 mg/patient per day, 250-1,250 mg/patient per day, or 500-1000 mg/patient per day. In some preferred embodiments, the dosage is 1000 mg/patient per day.

In a particular embodiment, the abiraterone is formulated as a prodrug such as abiraterone acetate. Following administration, abiraterone acetate is converted into the active form, abiraterone. It is believed this conversion is esterase-mediated and not dependent on CYP. Abiraterone acetate is a lipophilic compound with an octanol-water partition coefficient of 5.12 (Log P) and is practically insoluble in water. Abiraterone acetate is sold under the trade name ZYTIGA®. A tablet for oral administration includes 250 mg abiraterone acetate and inactive ingredients including colloidal silicon dioxide, croscarmellose sodium, lactose monohydrate, magnesium stearate, microcrystalline cellulose, povidone, and sodium lauryl sulfate. The recommend daily dosage is 1000 mg per day (e.g., four tablets of ZYTIGA®), but can be increased or decreased depending on the condition of the subject to be treated. For example, the dosage or dosing frequency is often decreased (e.g., to 250 mg/day, 500 mg/day, 750 mg/day, etc.,) if the subject is experiencing hepatotoxicity, or when the drug is co-administered with a CYP2D6 substrate. Alternatively, the dosage or dosing frequency can be increased when administered in combination with a strong CYP3A4 inducer.

Abiraterone acetate is also available as YONSA®. Each YONSA Tablet contains 125 mg of abiraterone acetate and inactive ingredients including lactose monohydrate, microcrystalline cellulose, croscarmellose sodium, sodium lauryl sulfate, sodium stearyl fumarate, butylated hydroxyanisole, and butylated hydroxytoluene. The recommended dosage of YONSA® is 500 mg (e.g., four 125 mg tablets), but this amount can be increased or decreased as needed. Typically, YONSA® is administered orally, once daily.

For the treatment of prostate cancer, abiraterone is often administered in combination with a steroid such as prednisone or prednisolone. The steroid is given to reduce the chances of (1) fluid retention, (2) raised blood pressure, or (3) decreased levels of potassium in the blood as a result of the abiraterone treatment. Suitable prednisone compositions and dosages for use in combination with abiraterone are known in the art. For example, a recommended prednisone co-therapy is 5 mg administered orally twice daily.

### Non-Steroidal Antiandrogens and Androgen Antagonists

In some embodiments, the antiandrogen or androgen antagonist is not a steroid or does not have a steroidal structure. In one embodiment, the compound has the structure:

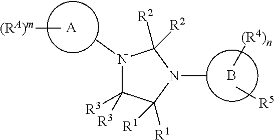

wherein,

ring A is monocyclic heteroaryl, bicyclic heteroaryl, or naphthyl;

m is 0, 1, 2, 3 or 4;

each R^{A} is independently selected from H, halogen, —CN, —NO₂, —OH, —OR⁹, —SR⁹, —S(═O)R¹⁰, —S(═O)₂R¹⁰, —N(R¹¹)S(═O)₂R¹⁰, —S(═O)₂N(R⁹)₂, —C(═O)R¹⁰, —OC(═O)R¹⁰, —CO₂R⁹, —N(R⁹)₂, —C(═O)N(R⁹)₂, substituted or unsubstituted C₁-C₆ alkyl, substituted or unsubstituted C₁-C₆ fluoroalkyl, substituted or unsubstituted C₁-C₆ fluoroalkoxy, substituted or unsubstituted C₁-C₆ alkoxy, substituted or unsubstituted C₁-C₆ heteroalkyl, substituted or unsubstituted C₃-C₁₀ cycloalkyl, substituted or unsubstituted C₂-C₁₀ heterocycloalkyl, substituted or unsubstituted phenyl or substituted or unsubstituted monocyclic heteroaryl;

each R¹ is independently selected from H, —OH, substituted or unsubstituted C₁-C₆ alkyl, substituted or unsubstituted C₁-C₆ alkoxy, and substituted or unsubstituted C₁-C₆ fluoroalkyl;

or both R¹ are taken together with the carbon atom to which they are attached to form a substituted or unsubstituted C₃-C₁₀ cycloalkyl or a substituted or unsubstituted C₂-C₁₀ heterocycloalkyl;

each R² is H; or both R² are taken together with the carbon to which they are attached to form —C(═S)— or —C(═O)—;

each R³ is H; or both R³ are taken together with the carbon to which they are attached to form —C(═S)— or —C(═O)—; provided that each R² is not H if each R³ is H;

ring B is phenyl, naphthyl, monocyclic heteroaryl, or bicyclic heteroaryl;

n is 0, 1, 2, 3 or 4;

each R⁴ is independently selected from H, halogen, —CN, —NO₂, —OH, —OR⁹, —SR⁹, —S(═O)R¹⁰, —S(═O)₂R¹⁰, —N(R₁₁)S(═O)₂R¹⁰, —S(═O)₂N(R⁹)₂, —C(═O)R¹⁰, —OC(═O)R¹⁰, —CO₂R⁹, —OCO₂R¹⁰, —N(R⁹)₂, —C(═O)N(R⁹)₂, —OC(═O)N(R⁹)₂, —NR¹¹C(═O)N(R⁹)₂, —NR¹¹C(═O)R¹⁰, —NR¹¹C(═O)OR¹⁰, substituted or unsubstituted C₁-C₆ alkyl, substituted or unsubstituted C₁-C₆ fluoroalkyl, substituted or unsubstituted C₁-C₆ fluoroalkoxy, substituted or unsubstituted C₁-C₆ alkoxy, and substituted or unsubstituted C₁-C₆ heteroalkyl;

R⁵ is substituted or unsubstituted C₂-C₁₀ alkyl, substituted or unsubstituted C₂-C₁₀ fluoroalkyl, substituted or unsubstituted C₂-C₁₀ alkoxy, substituted or unsubstituted C₂-C₁₀ fluoroalkoxy, substituted or unsubstituted C₂-C₁₀ heteroalkyl, substituted or unsubstituted C₂-C₁₀ heterofluoroalkyl, or -L¹-L²-R⁶;

L¹ is absent, —O—, —S—, —S(═O)—, —S(═O)₂—, —NH—, —C(═O)—, —C(═O)NH—, —NHC(═O)—, —NHC(═O)O—, —NHC(═O)NH—, —C(═O)O—, —OC(═O)—, —OC(═O)O—, —OC(═O)NH—, —NHS(═O)₂—, or —S(═O)₂NH—;

L² is substituted or unsubstituted C₁-C₆ alkylene, substituted or unsubstituted C₁-C₆ fluoroalkylene or substituted or unsubstituted C₁-C₆ heteroalkylene;

R⁶ is —CN, —NO₂, —OH, —OR⁹, —SR⁹, —S(═O)R¹⁰, —S(═O)₂R¹⁰, —N(R₁₁)S(═O)₂R¹⁰, —S(═O)₂N(R⁹)₂, —C(═O)R¹⁰, —OC(═O)R¹⁰, —CO₂R⁹, —OCO₂R¹⁰, —N(R⁹)₂, —C(═O)N(R⁹)₂, —OC(═O)N(R⁹)₂, —NR¹¹C(═O)N(R⁹)₂, —NR¹¹C(═O)R¹⁰, —NR¹¹C(═O)OR¹⁰, substituted or unsubstituted C.sub.1-C.sub.6alkyl, substituted or unsubstituted C₁-C₆ fluoroalkyl, substituted or unsubstituted C₁-C₆heteroalkyl, substituted or unsubstituted C₃-C₁₀ cycloalkyl, substituted or unsubstituted C₂-C₁₀ heterocycloalkyl, substituted or unsubstituted monocyclic heteroaryl, substituted or unsubstituted bicyclic heteroaryl, substituted or unsubstituted phenyl, or substituted or unsubstituted naphthyl;

each R⁹ is independently selected from H, substituted or unsubstituted C₁-C₆ alkyl, substituted or unsubstituted C₁-C₆ heteroalkyl, substituted or unsubstituted C₁-C₆ fluoroalkyl, substituted or unsubstituted C₃-C₁₀ cycloalkyl, substituted or unsubstituted C₂-C₁₀ heterocycloalkyl, substituted or unsubstituted aryl, substituted or unsubstituted heteroaryl, C₁-C₄ alkylene-(substituted or unsubstituted C₃-C₁₀ cycloalkyl), —C₁-C₄ alkylene-(substituted or unsubstituted C₃-C₁₀ heterocycloalkyl), —C₁-C₄ alkylene-(substituted or unsubstituted aryl), and —C₁-C₄ alkylene-(substituted or unsubstituted heteroaryl); or

two R⁹ groups attached to the same N atom are taken together with the N atom to which they are attached to form a substituted or unsubstituted C₂-C₁₀ heterocycloalkyl;

R¹⁰ is substituted or unsubstituted C₁-C₆ alkyl, substituted or unsubstituted C1-C6heteroalkyl, substituted or unsubstituted C₁-C₆ fluoroalkyl, a substituted or unsubstituted C₃-C₁₀ cycloalkyl, a substituted or unsubstituted C₂-C₁₀ heterocycloalkyl, a substituted or unsubstituted aryl, a substituted or unsubstituted benzyl, a substituted or unsubstituted heteroaryl, —C₁-C₄ alkylene-(substituted or unsubstituted C₃-C₁₀ cycloalkyl), —C₁-C₄ alkylene-(substituted or unsubstituted C₂-C₁₀ heterocycloalkyl), —C₁-C₄ alkylene-(substituted or unsubstituted aryl), or —C₁-C₄ alkylene-(substituted or unsubstituted heteroaryl);

R¹¹ is H or C₁-C₄ alkyl.

These compounds are described in U.S. Patent Application Publication No. 2013/0116258, which is incorporated herein by reference in its entirety.

2. Polo-Like Kinase Inhibitor

The combination therapies include one or more polo-like kinase (Plk) inhibitors. Polo-like kinases (Plks) are a family of conserved serine/threonine kinases involved in the regulation of cell cycle progression through G2 and mitosis. The catalytic domain of polo-like kinases is located in the N-terminus. The C-terminus of Plks contains one or two domains known as polo boxes that help localize the kinase to specific mitotic structures during mitosis. These include the centrosomes in early M phase, the spindle midzone in early and late anaphase and the midbody during cytokinesis.

Mammalian polo-like kinases include PLK1, Plk2/Snk, Plk3/Prk/FnK, Plk4/Sak, and Plk5. The polo-like kinase inhibitor can reduce or inhibit expression or activity of PLK1, Plk2/Snk, Plk3/Prk/FnK, Plk4/Sak, Plk5, and combinations thereof. For example, in some embodiments the inhibitor reduces or inhibits PLK1, Plk2/Snk, Plk3/Prk/FnK, Plk4/Sak, and/or Plk5 mRNA or protein expression. In some embodiments, the polo-like kinase inhibitor reduces or inhibits the kinase activity of PLK1, Plk2/Snk, Plk3/Prk/FnK, Plk4/Sak, and/or Plk5. In some embodiments, the polo-like kinase inhibitor reduces or inhibits expression of more than one polo-like kinase, for example by targeting a conserved region of the proteins, such as the polo box(es).

a. Small Molecule PLK Inhibitors

In a preferred embodiment, the polo-like kinase inhibitor (Plk inhibitor) is a small molecule. “Small molecule” refers to an organic molecule, inorganic molecule, or organometallic molecule having a molecular weight less than 2000, 1500, 1200, 1000, 750, or 500 atomic mass units. Polo-like kinase inhibitors are known in the art and include, for example, onvansertib, BI2536, volasertib (BI 6727), GSK461364, HMN-176, HMN-214, rigosertib (ON-01910), MLN0905, and Ro3280, several of which are discussed in Medema, et al., Clin. Cancer Res., 17:6459-6466 (2011), which is specifically incorporated by reference herein in its entirety.

Each of the Plk inhibitors, preferred dosages and routes of administration are discussed in more detail below, however, generally, the compounds can be administered to humans in an amount from about 0.0001 mg/kg of body weight to about 100 mg/kg of body weight per day. Generally, for intravenous injection or infusion, dosage may be lower than for other methods of delivery.

Some of the Plk inhibitors have been investigated for anti-cancer properties in preclinical experiments and clinical trials. In some embodiments, the dosage of Plk inhibitor used in combination therapies is the same as a dosage used to treat or prevent a cancer in a clinical trial, or a human equivalent to a dosage used to treat cancer in an animal study. However, it is believed that the more than additive effect of the combination therapy is not a generic consequence of G2/M arrest induced by the Plk inhibitor. Therefore, in some embodiments, the dosage is different than the dosage used to treat cancer. For example, the dosage can be lower than the dosage used to treat cancer, or the dosage can be higher than the dosage used to treat cancer provided that the dosage is safe and tolerable to the subject. Preferably, the dosage is at or below a maximum tolerated dose as determined in a clinical trial. In some embodiments, the maximum tolerated dose is 250 mg.

Onvansertib

In a preferred embodiment, the Plk inhibitor is onvansertib, also known as NMS-1286937 or NMS-P937, or a prodrug, analog, or derivative, or pharmaceutically acceptable salt thereof. Onvansertib has the structure:

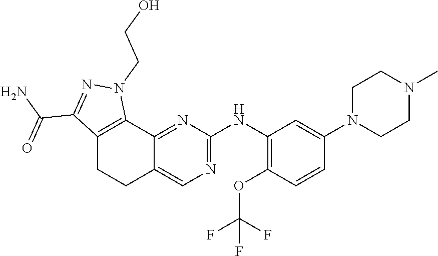 [SMILES: CN1CCN(c2ccc(OC(F)(F)F)c(Nc3ncc4c(n3)-c3c(c(C(N)=O)nn3CCO)CC4)c2)CC1]

Onvansertib is an orally bioavailable, highly specific small-molecule Polo-like kinase 1 (PLK1) inhibitor with potential antineoplastic activity. Preclinical evaluation has shown high potency of the compound in proliferation assays, displaying low nanomolar activity on a large number of cell lines, representative of both solid and hematological tumors. A phase 1 dose escalation study of onvansertib administered to adult patients with advanced/metastatic solid tumors has been completed (Hartsink-Segers, et al., Haematologica. 98(10):1539-46 (2013)).

Ongoing clinical trials of onvansertib include a Phase 1b/2 study of onvansertib in combination with FOLFIRI and Bevacizumab for second line treatment of metastatic colorectal cancer in patients with a Kras mutation (see ClinicalTrials.gov Identifier: NCT03829410). The proposed treatment for Phase 1b is an escalating starting dose of onvansertib of 12 mg/m² orally on days 1 through 5 every 14-days over two treatment courses (1 cycle) in combination with FOLFIRI (180 mg/m2 irinotecan, 400 g/m2 leucovorin, 400 mg/m2 bolus 5-fluorouracil (5-FU), and 2400 mg/m² continuous intravenous infusion 5-FU) and 5 mg/kg bevacizumab.

Another ongoing clinical trial is a Phase 2 study of onvansertib in combination with abiraterone and prednisone in adult patients with metastatic castration-resistant prostate cancer (see ClinicalTrials.gov Identifier: NCT03414034). In this study, onvansertib is administered orally once daily (QD) at a dose of 24 mg/m² for 5 days (day 1 through day 5) out of a 14-day cycle or at a dose of 12 mg/m² for 14 days (day 1 through day 14) out of a 21-day cycle. In both regimens, beginning on day 1 and continuing uninterrupted throughout each cycle, patients also receive abiraterone and prednisone. It is contemplated that any of the above clinical trial dosages and regimens can be used in the disclosed methods. In some preferred embodiments, onvansertib is administered at a dose of 12 mg/m², 18 mg/m², or 24 mg/m² daily (e.g., days 1-5 of a 14 day cycle, days 1-5 of a 21 day cycle, or days 1-14 of a 21 day cycle).

Dihydropteridinones

In some embodiments, the Plk inhibitor has the formula described in U.S. Pat. No. 6,806,272, which is incorporated herein reference in its entirety. The compound can have the formula:

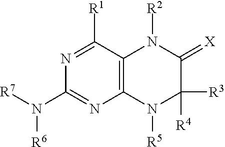

wherein

R¹ denotes a group selected from among hydrogen, NH₂, XH, halogen and a C₁-C₃ -alkyl group optionally substituted by one or more halogen atoms,

R₂ denotes a group selected from among hydrogen, CHO, XH, —X—C₁-C₂-alkyl and an optionally substituted C₁-C₃-alkyl group,

R³, R⁴ which may be identical or different denote a group selected from among optionally substituted C₁-C₁₀-alkyl, C₂-C₁₀-alkenyl, C₂-C₁₀-alkynyl, aryl, heteroaryl, C₃-C₈-cycloalkyl, C₃-C₈-heterocycloalkyl, —X-aryl, —X-heteroaryl, —X-cycloalkyl, —X-heterocycloalkyl, —NR⁸-aryl, —NR⁸-heteroaryl, —NR⁸-cycloalkyl and —NR⁸-heterocycloalkyl, or a group selected from among hydrogen, halogen, COXR⁸, CON(R⁸)₂, COR⁸ and XR⁸, or R³ and R⁴ together denote a 2- to 5-membered alkyl bridge which may contain 1 to 2 heteroatoms,

R⁵ denotes hydrogen or a group selected from among optionally substituted C₁-C₁₀-alkyl, C₂-C₁₀-alkenyl, C₂-C₁₀-alkynyl, aryl, heteroaryl and —C₃-C₆-cycloalkyl, or R³ and R⁵ or R⁴ and R⁵ together denote a saturated or unsaturated C₃-C₄-alkyl bridge which may contain 1 to 2 heteroatoms, R⁶ denotes optionally substituted aryl or heteroaryl, R⁷ denotes hydrogen or —CO—X—C₁-C₄-alkyl, and X in each case independently of one another denotes O or S, R⁸ in each case independently of one another denotes hydrogen or a group selected from among optionally substituted C₁-C₄-alkyl, C₂-C₄-alkenyl, C₂-C₄ alkynyl and phenyl,

optionally in the form of the tautomers, the racemates, the enantiomers, the diastereomers and the mixtures thereof, and optionally the pharmacologically acceptable acid addition salts thereof.

Specific compounds of the formula above and other Plk inhibitors are described below.

BI2536

In a preferred embodiment, the Plk inhibitor is BI2536, or a prodrug, analog, or derivative, or pharmaceutically acceptable salt thereof. BI2536 has the structure:

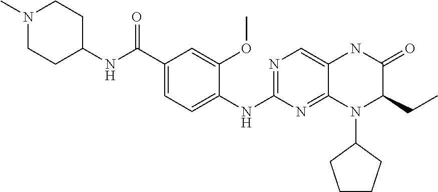 [SMILES: CC[C@@H]1C(=O)Nc2cnc(Nc3ccc(C(=O)NC4CCN(C)CC4)cc3OC)nc2N1C1CCCC1]

BI2536 is a potent PLK1 inhibitor with IC50 of 0.83 nM (Steegmaier, et al., Current Biology, 17:316-322 (2007)). It shows 4- and 11-fold greater selectivity against Plk2 and Plk3. In preclinical experiments, BI2536 given i.v. once or twice per week was highly efficacious in diverse xenograft models with acceptable tolerability. It is believed the drug inhibited cell proliferation through a mitotic arrest, and subsequently induction of tumor-cell death. Administration of BI2536 at 50 mg/kg once or twice per week significantly inhibited growth of HCT 116 xenografts with T/C of 15% and 0.3%, respectively. BI2536 treatment twice-weekly also lead to excellent tumor-growth in BxPC-3 and A549 models with T/C of 5% and 14%, respectively ((Steegmaier, et al., Current Biology, 17:316-322 (2007)).

BI2536 has been the subject of a number of clinical trials testing the safety and efficacy of the drug in a range of dosages and regimes and for treatment of a number of cancers. For example, in a randomized, open-label, phase I/II trial to investigate the maximum tolerated dose of the Polo-like kinase inhibitor BI2536 in elderly patients with refractory/relapsed acute myeloid leukemia, 68 elderly patients with relapsed/refractory AML were administered BI2536 on one of three schedules (day 1, days 1-3, and days 1+8). The maximum tolerated dose was 350 mg and 200 mg in the day 1 and days 1+8 schedules, respectively. The day 1-3 schedule appeared equivalent to the day 1 schedule and was discontinued early (Muller-Tidow, et al., Br. J. Haematol., 163(2):214-22 (2013)). Likewise, a phase I open-label dose-escalation study tested the maximum tolerated dose of intravenous BI2536 together with pemetrexed in previously treated patients with non-small-cell lung cancer. The patients received 500 mg/m² pemetrexed and escalating doses of BI2536 on day 1 every 3 weeks. Forty-one patients received BI2536 (100-325 mg). Two dose-limiting toxicities (DLT) occurred at BI2536 325 mg (grade 3 pruritus and rash; grade 4 neutropenia). Therefore, the maximum tolerated dose (MTD) for BI2536 in combination with pemetrexed was 300 mg (Ellis, et al., Clin. Lung Cancer, 14(1):19-27 (2013) Epub 2012 Jun. 1). BI2536 at 200 mg combined with standard-dose pemetrexed was determined to have an acceptable safety profile. Other studies have suggested a lower MTD, e.g., 50-70 mg (Frost, et al., Curr. Oncology, 19(1):e25-35 (2012)).

An open, randomized, clinical phase II trial in patients with un-resectable advanced pancreatic cancer investigated the efficacy, safety, and pharmacokinetics of BI 2536 administered in repeated 3-week cycles as a single i.v. dose of 200 mg on day 1 or as 60 mg doses on days 1, 2, and 3.

Therefore, in some embodiments, BI2536 is administered to a subject 1, 2, 3, or more times a week in a dosage of about 1-500 mg, preferably about 10-400 mg, more preferably about 50-350 mg, most preferably 60-300 mg. In a particular embodiment the dosage is 50, 100, 150, 200, 250, 300, or 350 mg of BI2536 administered to a subject once, twice, three times or more than three times a week, or once every two, three or four weeks. In some embodiments, BI2536 is administered by intravenous injection or infusion.

Volasertib (BI6727)

Like BI2536, BI6727 is an ATP-competitive kinase inhibitor from the dihydropteridinone class of compounds. BI6727 is a highly potent PLK1 inhibitor with IC50 of 0.87 nM. It also shows 6- and 65-fold greater selectivity against Plk2 and Plk3. BI6727 at concentrations up to 10 μM displays no inhibitory activity against a panel of >50 other kinases in vitro (Rudolph D, et al. Clin. Cancer Res., 15(9), 3094-3102 (2009)). BI6727 has the structure:

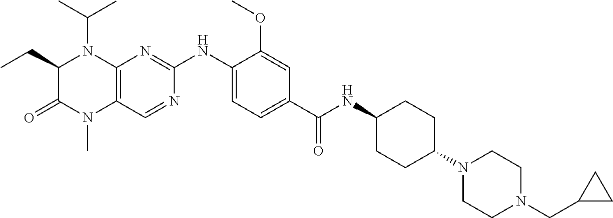 [SMILES: CC[C@@H]1C(=O)N(C)c2cnc(Nc3ccc(C(=O)N[C@H]4CC[C@H](N5CCN(CC6CC6)CC5)CC4)cc3OC)nc2N1C(C)C]

Preclinical experiments in a mouse model show that administration of BI6727 at ˜25 mg/kg/day significantly inhibits the growth of multiple human carcinoma xenografts including HCT116, NCI-H460, and taxane-resistant CXB1 colon carcinoma, accompanied by an increase in the mitotic index as well as an increase in apoptosis (Rudolph D, et al. Clin. Cancer Res., 15(9), 3094-3102 (2009)). Some in vivo studies indicate that BI6727 exhibits a better toxicity and pharmacokinetic profile than BI2536 (Harris, et al., BMC Cancer, 12, 80 (2012)).

BI6727 has been the subject of a number of clinical trials testing the safety and efficacy of the drug in a range of dosages and regimes and for treatment of a number of cancers. A phase I first-in-humans study of volasertib was conducted in 65 patients with advanced solid tumors, including 10 with NSCLC. Volasertib was administered i.v. once every 3 weeks following a dose-escalation design (12-450 mg). The study reported neutropenia, thrombocytopenia, and febrile neutropenia as DLTs and an MTD of 400 mg (Gil, et al., J. Clin. Oncol., 28 Suppl 15:abstr 3061 (2010), Schoffski, et al., Eur. J. Cancer, 48(2):179-86 (2012)). 300 mg was the recommended dose for further development based on overall tolerability. In a phase I study of volasertib (BI 6727) combined with afatinib (BIBW 2992) in advanced solid tumors, the MTD was determined to be 300 mg of BI 6727, when administered in combination with afatinib (Peeters, et al., J. Clin. Oncol., 31 (suppl; abstr 2521) (2013)).

Therefore, in some embodiments, BI 6727 is administered to a subject 1, 2, 3, or more times a week in a dosage of between about 1-600 mg, preferably about 10-500 mg, more preferably about 50-400 mg, most preferably 100-350 mg. In a particular embodiment the dosage is 50, 100, 150, 200, 250, 300, 350, or 400 mg of BI 6727 administered to a subject once, twice, three times or more than three times a week, or once every two, three or four weeks. In some embodiments, BI 6727 is administered by intravenous injection or infusion.

### Other Classes of Plk Inhibitors

The Plk inhibitor may be a molecule other than onvansertib or a dihydropteridinone. Other classes of inhibitors include, but are not limited to, pyridopyrimidines (see U.S. Patent Application Publication No. 2010/004141 and WO 2009/112524), aminopyrimidines (see U.S. Patent Application Publication No. 2010/010014), substituted thiazolidinones (see European Patent Application No. EP 2141163), pteridine derivatives (see European Patent Application No. EP 2079743), dihydroimidazo[1,5-f]pteridines (see WO 2010/025073), metasubstituted thiazolidinones, (see U.S. Patent Application Publication No. 2010/048891), benzyl styryl sulfone analogues (see WO 2009/128805), and stilbene derivatives.

The applications cited above are incorporated herein by reference in their entirety.

Specific inhibitors are discussed below:

GSK461364

GSK461364 inhibits purified PLK1 with Ki of 2.2 nM. It is more than 1000-fold selective against Plk2/3.

The structure for GSK461364 is

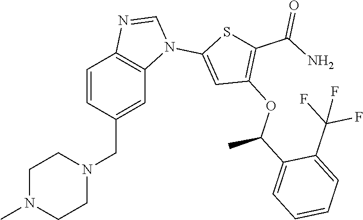 [SMILES: C[C@@H](Oc1cc(-n2cnc3ccc(CN4CCN(C)CC4)cc32)sc1C(N)=O)c1ccccc1C(F)(F)F]

Cell culture growth inhibition by GSK461364 can be cytostatic or cytotoxic but leads to tumor regression in xenograft tumor models under proper dose scheduling. In an animal model, dosages of 25, 50, and 100 mg/kg were administered via i.p. every 2 days or every 4 days (Gilmartin, et al., Cancer Res, 69(17), 6969-6977 (2009)).

A phase I first-in-humans study of GSK461364 was conducted in 27 patients with advanced solid tumors (Olmos, et al., Clin. Cancer Res., 17:3420-30 (2011)). The agent was administered i.v. following 2 schedules with different dosing (50-225 mg on days 1, 8, and 15 (schedule A) or 25-100 mg on days 1, 2, 8, 9, 15, and 16 (schedule B) on a 28-day cycle. DLTs included grade 4 neutropenia, sepsis, and pulmonary embolism. The final recommended phase II dose for GSK461364 was 225 mg administered intravenously in schedule A. Because of the high incidence (20%) of venous thrombotic emboli (VTE), co-administration of a prophylactic anticoagulation agent is recommended.

Therefore, in some embodiments, GSK461364 is administered to a subject 1, 2, 3, or more times a week in a dosage of between about 1-400 mg, preferably about 10-350 mg, more preferably about 25-300 mg, most preferably 25-225 mg. In a particular embodiment the dosage is 50, 100, 150, 200, 250, 300, 350, or 400 mg of GSK461364 administered to a subject once, twice, three times or more than three times a week, or once every two, three or four weeks. In some embodiments, GSK461364 is administered by intravenous injection or infusion.

HMN-176 and HMN-214

HMN-176 is a stilbene derivative that is an active metabolite of the prodrug HMN-214. It does not directly inhibit the enzymatic activity of PLK1 but rather affects subcellular distribution of PLK1. The structures of HMN-176 and HMN-214 are (A) and (B) respectively:

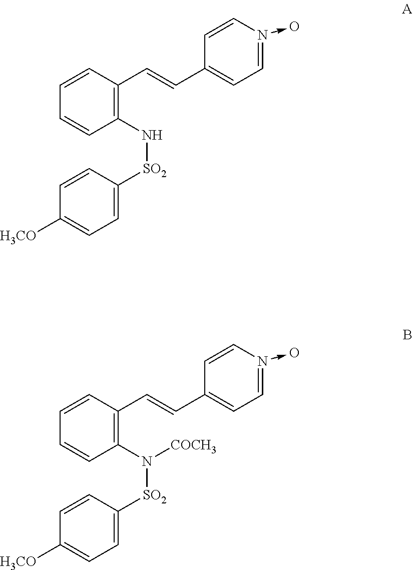 [SMILES: COc1ccc(S(=O)(=O)Nc2ccccc2/C=C/c2cc[n+]([O-])cc2)cc1 ; COc1ccc(S(=O)(=O)N(C(C)=O)c2ccccc2/C=C/c2cc[n+]([O-])cc2)cc1]

HMN-176 shows potent cytotoxicity toward various human tumor cell lines, and in mitotic cells, it causes cell cycle arrest at M phase through the destruction of spindle polar bodies, followed by the induction of DNA fragmentation. In preclinical experiments it was a potent antitumor activity in mouse xenograft models when administered at dosage of 10 mg/kg and 20 mg/kg on days 1 and 28 (Tanaka, et al., Cancer Res., 63:6942-6947 (2003)).

A phase I pharmacokinetic study of HMN-214 in patients with advanced solid tumors, thirty-three patients were enrolled onto four dosing cohorts of HMN-214 from 3 to 9.9 mg/m²/d using a continuous 21-day dosing schedule every 28 days. A severe myalgia/bone pain syndrome and hyperglycemia were dose-limiting toxicities at 9.9 mg/m²/d, and the maximum tolerated dose and recommended dose on this schedule was determined to be 8.0 mg/m²/d (Garland, et al., Clin. Cancer Res., 1;12(17):5182-9 (2006)).

In another study, DLTs of prolonged neutropenia, febrile neutropenia, neutropenic sepsis, electrolyte disturbance, neuropathy, and myalgia were observed at doses of 24 to 48 mg/m² for 5 consecutive days every 4 weeks. MTD was established at the range of 18 to 30 mg/m², based on previous patient treatment load (Patnaik, J. Clin. Oncol., 22 Suppl:abstr 514.).

Therefore, in some embodiments, HMN-214 (or HMN-176) is administered to a subject 1, 2, 3, 4, 5, 6, or 7 times a week in a dosage of between about 1-100 mg/m², preferably about 2.5-50 mg/m², more preferably about 3-40 mg/m², most preferably 7.5-30 mg/m². In a particular embodiment the dosage is 1, 2, 3, 4, 5, 6, 7, 8, 9, or 10 mg/m² of HMN-214 (or HMN-176) administered to a subject once, twice, three times or more than three times a week, for example, on days 1-21 of a 28 day cycle. In another particular embodiment the dosage is 10 to 48 mg/m² preferably 18 to 30 mg/m² of HMN-214 (or HMN-176) administered once, twice, three times or more than three times a week, for example, days 1-5 of a 28 day cycle. In some embodiments, HMN-214 (or HMN-176) is administered orally.

Rigosertib (ON-01910)

The benzyl styryl sulfone analogue ON 01910 is an ATP-noncompetitive, multitargeted inhibitor of several tyrosine kinases and cyclin-dependent kinase 1 (Cdk1; IC50=18-260 nmol/L). It is reported to have a particularly strong potency (IC50=9-10 nmol/L) toward PLK1 (Gumireddy, et al., Cancer Cell, 7:275-86 (2005)). The structure of ON-01910 is

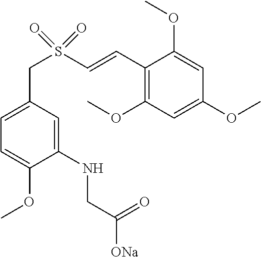

In preclinical animal studies in mouse xenograft models of Bel-7402, MCF-7, and MIA-PaCa cells, Rigosertib (250 mg/kg) inhibited tumor growth and (200 mg/kg) showed inhibition of tumor growth in a mouse xenograft model of BT20 cells (Gumireddy, et al., Cancer Cell, 7:275-86 (2005), Reddy, et al., J. Med. Chem., 54(18), 6254-6276 (2011)).

A phase I first-in-humans study of ON 01910 was conducted in 20 patients with advanced solid tumors (none with NSCLC). The agent was administered i.v. at 80 to 4,370 mg by accelerated titration design on days 1, 4, 8, 11, 15, and 18 in 28-day cycles (Jimeno, et al., J. Clin. Oncol., 26:5504-10 (2008)). Grade 3 abdominal pain was reported as a DLT at an MTD of 3,120 mg.

In a clinical trial testing the safety and pharmacokinetics of oral ON 01910 in patients with myelodysplastic syndrome, ON 01910 was given twice a day up to 14 days at doses escalating from 70 mg to 700 mg.

Therefore, in some embodiments, ON 01910 is administered to a subject 1, 2, 3 or more days a week in a dosage of about 50-6,000 mg, preferably about 60-4,500 mg, more preferably about 150 mg-1,500 mg once daily, or 75-750 mg twice daily. In particular embodiments, ON 01910 administered to a subject once, twice, three times or more than three times a week, or once every two, three or four weeks. In a specific embodiment, the drug is administered every day for 14 days. In some embodiments, ON 01910 is administered by intravenous injection or infusion.

MLN0905

MLN0905 is a potent inhibitor of PLK1 with IC50 of 2 nM. MLN0905 inhibits cell mitosis with EC50 of 9 nM and Cdc25C-T96 phosphorylation, a direct readout of PLK1 inhibition, with EC50 of 29 nM (Duffey, Med Chem, 55(1), 197-208 (2012)). The structure of MLN0905 is

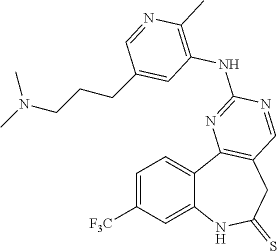 [SMILES: Cc1ncc(CCCN(C)C)cc1Nc1ncc2c(n1)-c1ccc(C(F)(F)F)cc1NC(=S)C2]

In preclinical experiments indicate an effective dosage range of about 1 mg/kg-50 mg/kg. One study indicates a preferred dosage of about 3-15 mg/kg with a maximum tolerated dose on QD (daily) schedule to be 6.25 mg/kg and on the QD×3/wk (3-days on/4-days off) schedule to be 14.5 mg/kg (Shi, et al., Mol. Cancer Thera., 11(9), 2045-2053 (2012)).

RO3280

RO3280 is a potent, highly selective inhibitor of Polo-like kinase 1 (PLK1) with IC50 of 3 nM. The structure of RO3280 is

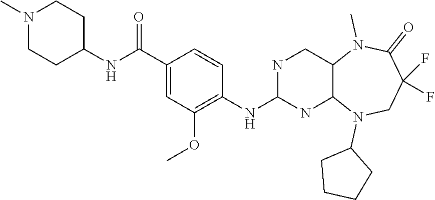 [SMILES: COc1cc(C(=O)NC2CCN(C)CC2)ccc1NC1NCC2C(N1)N(C1CCCC1)CC(F)(F)C(=O)N2C]

RO3280 shows the strong anti-proliferative activity against lung cancer cell line H82, colorectal cancer cell HT-29, breast cancer cell MDA-MB-468, prostate cancer cell PC3 and skin cancer cell A375 with IC50s of 5, 10, 19, 12 and 70 nM, respectively. RO3280 also showed promising antitumor activity in nude mouse implanted with HT-29 human colorectal tumors ranging from 72% tumor growth inhibition when dosed once weekly at 40 mg/kg, to complete tumor regression when dosed more frequently (Chen, et al., Bioorg. Med. Chem. Lett., 22(2), 1247-1250 (2012).

TAK-960

TAK-960 is an orally bioavailable, potent, and selective PLK1 inhibitor that has shown activity in several tumor cell lines, including those that express multidrug-resistant protein 1 (MDR1) (Hikichi, et al., Mol Cancer Ther. 11(3):700-9 (2012)). A Phase 1, open-label, dose-escalation study of orally administered TAK-960 has been completed.

CFI-400945 Fumarate

CFI-400945 is an inhibitor of polo-like kinase 4 (PLK4). Many tumors are shown to make too much PLK4. Phase 1 clinical trials have orally administered CFI-400945 fumarate at dose levels of 3, 6, 11, 16, 24, and 32 mg/day are currently underway (Mason, et al., Cancer Cell, V 26(2), pp.163-176 (2014)).

b. Functional Nucleic Acid Inhibitors of PLK

In some embodiments, the polo-like kinase inhibitor is a functional nucleic acid that targets PLK1, Plk2/Snk, Plk3/Prk/FnK, Plk4/Sak, and/or Plk5. The functional nucleic acid can be, for example, an antisense molecule, aptamer, ribozyme, triplex forming oligonucleotide, external guide sequence, or RNAi that targets inhibits or reduces expression or translation of PLK1, Plk2/Snk, Plk3/Prk/FnK, Plk4/Sak, and/or Plk5 mRNA.

TKM-080301

In a particular embodiment, the Functional Nucleic Acid Inhibitor of PLK is TKM-080301. TKM-080301 has been effective when given in a 30-minute intravenous infusion. Clinical trials have administered doses ranging from 0.15 mg/kg per week to 0.9 mg/kg per week. Dose-limiting toxicities were observed at the 0.9 mg/kg per-week dose.

Other suitable Plk (e.g., PLK1) inhibitors include, without limitation, SBE-13, ZK-Thiazolidinone, BI-4834 (a dihydropteridinone like BI2536 and volasertib), CAP-53194, Cyclapolin-9, DAP-81, GW843682X, MK-1496, PHA-680626, T521, UMB103, and UMB160.

In some embodiments, the Plk inhibitor does not inhibit the kinase activity of the Plk (e.g., PLK1), but rather, blocks its polo-box domain function. Exemplary polo-box domain blockers include Poloxin, Poloxin-2, Poloxime, Poloxipan, and Thymoquinone.

3. Additional Therapeutic or Prophylactic Agents

In some embodiments, the combination therapy includes additional agents. In addition to one or more antiandrogen or androgen antagonist, and one or more Plk inhibitors, the combination therapies can include therapeutic or prophylactic agents to be co-administered with an antiandrogen or androgen antagonist, or with a Plk inhibitor. For example, abiraterone acetate is routinely administered in combination with a steroid such as prednisone or prednisolone. Therefore, in some embodiments, the combination includes prednisone or prednisolone.

In some embodiments, the combination therapy includes administration of an additional antiandrogen therapeutic agent, an immunotherapeutic agent, an agent for treating bone complications, a chemotherapeutic agent that is not considered an antiandrogen or PLK inhibitor, an agent that stimulates production of white-blood cells (for example granulocyte colony-stimulating factor or granulocyte-macrophage colony-stimulating factor), a prophylactic anticoagulation agent, or a combination thereof. In some embodiments, the combination therapy includes administration of an additional first or second line therapeutic agent for treatment of CRPC, such as one or more of the agents reviewed in Shapiro and Tareen, Expert Rev. Anticancer Ther., 12(7):951-964 (2012), Heidegger, et al., J. Steroid Biochem. Mol. Biol., 138(100): 248-256 (2013), and Lui, et al., Cancer Control, 20(3):181-187 (2013), each of which is specifically incorporated by reference herein in its entirety.

In some embodiments, the additional active agent is administered to the subject during the same cycle as the antiandrogen or androgen antagonist and Plk inhibitor. For example, the combination therapy can include a Plk inhibitor, abiraterone, and a second antiandrogen or androgen antagonist. In a particular embodiment, the second antiandrogen or androgen antagonist is enzalutamide or ARN-509 (apalutamide) which are androgen receptor inhibitors (Efstathiou, et al., European Cancer Congress 2013, September 27-1, 2013, Amsterdam, The Netherlands, Abstract 2854).

It is appreciated that some combinations should not be administered simultaneously. Such combinations are preferably administered in series, for example using cycles, or drug holidays. For example, sipuleucel-T, sold under the trade name PROVENGE®, is an immune therapy that stimulates that immune system to attack the cancer. It is typically not advised to simultaneously administer abiraterone acetate and sipuleucel-T because abiraterone is typically administered simultaneously with a steroid. However, sequential administration of sipuleucel-T and abiraterone acetate has been proposed.

B. Formulations

Formulations of and pharmaceutical compositions including one or more active agents are provided. The combination therapies can include administration of the active agents together in the same admixture, or in separate admixtures. Therefore, the pharmaceutical compositions can include an antiandrogen or androgen antagonist, Plk inhibitor, or a combination thereof. In some embodiments, the pharmaceutical compositions can include one or more additional active agents. Therefore, in some embodiments, the pharmaceutical composition includes two, three, or more active agents. The pharmaceutical compositions can be formulated as a pharmaceutical dosage unit, referred to as a unit dosage form. Such formulations typically include an effective amount of an antiandrogen or androgen antagonist, a Plk inhibitor, or a combination thereof. Effective amounts of the active agents are discussed in more detail below. It will be appreciated that in some embodiments the effective amount of antiandrogen or androgen antagonist, or Plk inhibitor in a combination therapy is different from that amount that would be effective for the antiandrogen or androgen antagonist, or Plk inhibitor to achieve the same result individually. For example, in some embodiments the effective amount of antiandrogen or androgen antagonist, or Plk inhibitor, is a lower dosage of the antiandrogen or androgen antagonist, or Plk inhibitor in a combination therapy than the dosage of the antiandrogen or androgen antagonist, or Plk inhibitor that is effective when one agent is administered without the other. Alternatively, in some embodiments the effective amount of antiandrogen or androgen antagonist, or Plk inhibitor, is a higher dosage of the antiandrogen or androgen antagonist, or Plk inhibitor in a combination therapy than the dosage of the antiandrogen or androgen antagonist, or Plk inhibitor that is effective when one agent is administered without the other. In other embodiments, the dosage of one agent is higher and the dosage of the other agent is lower than when one agent is administered without the other. In some case, the agents are not effective when administered alone, and only effective when administered in combination.

The active agents can be administered and taken up into the cells of a subject with or without the aid of a delivery vehicle. Appropriate delivery vehicles for the active agents are known in the art and can be selected to suit the particular inhibitor. For example, in some embodiments, the active agent(s) is incorporated into or encapsulated by a nanoparticle, microparticle, micelle, synthetic lipoprotein particle, or carbon nanotube.

Pharmaceutical compositions including active agent(s) with or without a delivery vehicle are provided. The pharmaceutical compositions can be formulated for parenteral, enteral, or transmucosal routes of administration. Appropriate carriers that can be included in the compositions for parenteral or enteral delivery are standard in the art and can be readily determined by a person of ordinary skill in the art.

C. Adjunct and Additional Therapies and Procedures

The combination therapies can be administered to a subject in combination with one or more adjunct therapies or procedures, or can be an adjunct therapy to one or more primary therapies or producers. The additional therapy or procedure can be simultaneous or sequential with the combination therapy. In some embodiments, the additional therapy is performed between drug cycles or during a drug holiday that is part of the combination therapy dosage regime. In preferred embodiment, the additional therapy is a conventional treatment for cancer, more preferably a conventional treatment for prostate or breast cancer, most preferably a conventional treatment for hormone-resistant prostate or breast cancer. For example, in some embodiments, the additional therapy or procedure is surgery, a radiation therapy, or chemotherapy. For example, in a particular embodiment, combination therapies are used simultaneously or sequentially with a regime of a chemotherapeutic agent, e.g., docetaxel or cabazitaxel. In some embodiments, the adjunct or additional therapy is part of the combination therapy.

### IV. Kits

The active agents and compositions, reagents, and other materials can be packaged together in any suitable combination as a kit useful for performing, or aiding in the performance of, the methods. It is useful if the kit components in a given kit are designed and adapted for use together in the method. For example, kits with one or more compositions for administration to a subject, may include a pre-measured dosage of the composition in a sterile needle, ampule, tube, container, or other suitable vessel or oral or injectable administration The kits may include instructions for administering the compounds (e.g., dosages and dosing regimens).

In some embodiments, the kits can include, for example, a dosage supply of an antiandrogen or androgen antagonist, a polo-like kinase inhibitor, or a combination thereof separately or together in the same admixture. The active agents can be supplied alone (e.g., lyophilized), or in a pharmaceutical composition. The active agents can be in a unit dosage, or in a stock that should be diluted prior to administration. In some embodiments, the kit includes a supply of pharmaceutically acceptable carrier. The kit can also include devices for administration of the active agents or compositions, for example, syringes. The kits can also include reagents for isolation of samples from a subject (e.g., needles, syringes, scalpels). In some embodiments, the kits include reagents for detecting or analyzing gene expression for a sample. For example, the kits can include PCR reagents, in-situ hybridization reagents, flow cytometry reagents, antibodies, primers, and combinations thereof. Preferably, the kits include instructional materials. The instructional material can include a publication, a recording, a diagram, or any other medium of expression which can be used to communicate the usefulness of the compositions and methods of the kit. For example, the instructional material may provide instructions for methods using the kit components, such as performing gene expression analysis and drug administration.

It is to be understood that the methods and compositions are not limited to specific synthetic methods, specific analytical techniques, or to particular reagents unless otherwise specified, and, as such, can vary. It is also to be understood that the terminology used is for the purpose of describing particular embodiments only and is not intended to be limiting.

The present invention will be further understood by reference to the following non-limiting examples.

### EXAMPLES

### Example 1

### Combined Treatment with PLK1 Inhibitors and Abiraterone Exerts an AR-Independent, More than Additive Effect Across Different Cancer Cells

Prostate cancer, the second leading cause of cancer-related death amongst men in the United States, is driven by the Androgen Receptor (AR), a member of the nuclear receptor superfamily. Therapeutic castration is effective at slowing down progression of this disease, however, for many patients the cancer recurs as castrate-resistant prostate cancer (CRPC).

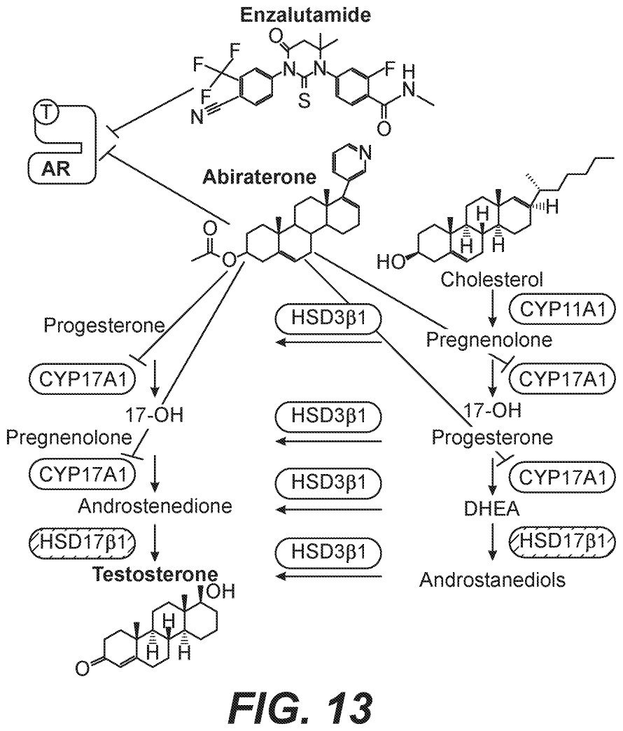

Abiraterone acetate, a current standard of care for CRPC, inhibits residual testosterone synthesis in adrenal glands and in the tumor cells themselves. It also inhibits the AR pathway by directly antagonizing AR (similarly to the antiandrogen Enzalutamide). See FIGS. 12 and 13. Unfortunately, over time (˜9-15 months) CRPC develops resistance to antiandrogen therapy.

Onvansertib is a highly specific and orally available PLK1 inhibitor with a 24-hour half-life. Tumor cells are 10-fold more sensitive to Onvansertib induced cell death than normal cells.

### Materials and Methods

Culturing of Cells

All cell lines were cultured in a 37° C. humidified incubator supplied with 5% CO₂, were maintained subconfluent and used for no more than 20 passages. All media was supplemented with either fetal bovine serum (FBS) or charcoal-stripped FBS (csFBS) where indicated, contained 2 mM Glutamine and lacked antibiotics. C4-2, LNCaP and PSN-1 were grown in RPMI-1640 with 10% FBS. Panc 10.05 was grown in RPMI-1640 with 15% FBS and 10 IU/ml human recombinant insulin (Gibco). OAW28 was grown in Advanced DMEM (Gibco) also supplemented with insulin. SK-OV-3 cells were grown in McCoy's 5A with 10% FBS. MV-4-11 cells were grown in IMDM with 10% FBS. OCI-AML-3 cells were grown in MEM with 20% FBS.

Measurements of Drug Sensitivity and Greater than Additive Effects of Drug Combinations

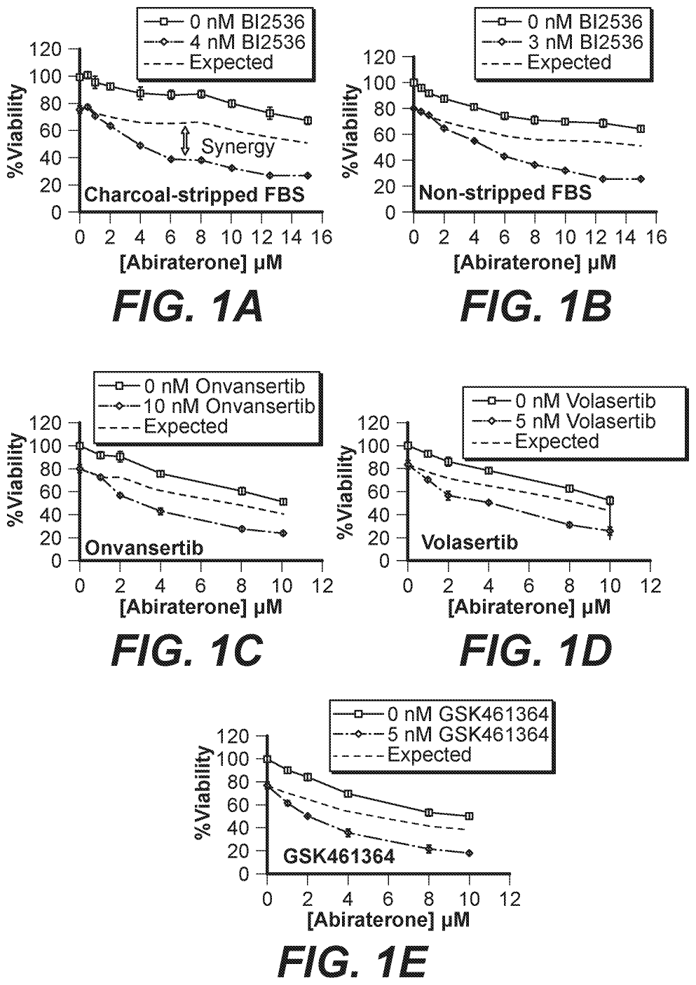

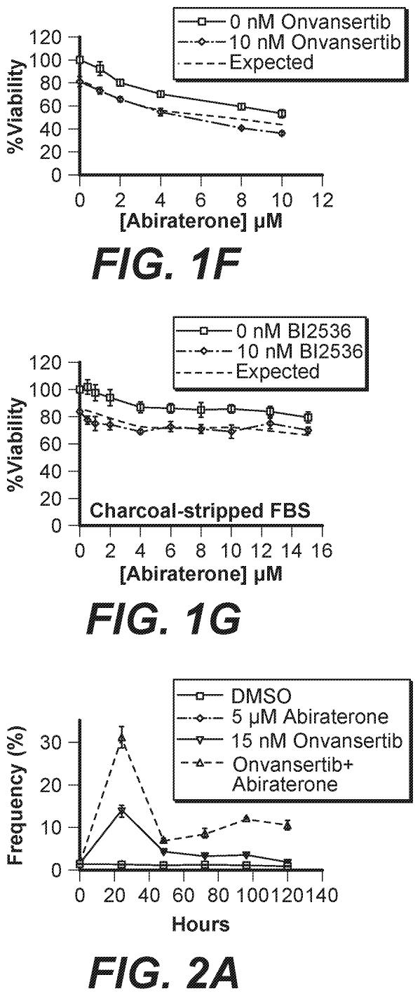

For dose response experiments in FIGS. 1A-1G, 3, 4A-4D, 5A-5F, cells were plated in 384-well plates in a total volume of 22.5 μl. C4-2 and LNCaP cells were plated at a density of 1,500 cells per well, MV-4-11 and OCI-AML-3 cells were plated at a density of 10,000 cells per well, all other cell lines were plated at 1,000 cells per well. The following day the indicated drugs, dissolved in DMSO, were diluted in the appropriate cell growth media while maintaining a constant final concentration of DMSO and added to the 384-well plates. Cells were incubated with drugs for 72 hours prior to measurement of viability using CellTiter-Glo™ (Promega) Luminescent Cell Viability Assay according to the manufacturer's recommendations. Luminescence was measured using Infinite™ M200 Pro plate reader (Tecan Group Ltd.). Using these data, viability relative to DMSO control was calculated and compared to the expected additive response calculated according to the Bliss Independence model of drug additivity (Bliss. Annals of Applied Biology, 26(3): 585-615 (1939)).

Identification of Mitotic and Apoptotic Cells by Flow Cytometry

C4-2 CRPC cells were plated in 6-well plates at a density of 300,000 cells per well and a total volume of 3 mls. The following day the indicated drugs were diluted in the appropriate growth media while maintaining a constant final concentration of DMSO and added to the wells. After the indicated amount of time cells were harvested by trypsinization. The media, trypsin and PBS wash were collected together to avoid loss of loosely attached or detached cells. Cells were fixed in 4% formaldehyde in PBS for 15 minutes, washed with PBS containing 1% bovine serum albumin (PBS-BSA), and then stored in methanol at −20° C. overnight. Cells were then washed twice in PBS-BSA 0.1% Tween-20, incubated with primary antibodies overnight at 4° C., washed with PBS-BSA 0.1% Tween-20 and incubated for 1 hour with fluorescent-dye conjugated secondary antibodies (diluted 1:200, Alexa Fluor, Molecular Probes) at room temperature for 1 hour. Primary antibodies included anti-phospho-serine 10 histone H3 (clone 3H10, Millipore) and anti-active caspase-3 (clone C92-605, BD Pharmingen). The fixed cells were then washed with PBS-BSA 0.1% Tween-20 and resuspended in PBS containing 1 μg/ml 4,6-diamidino-2-phenylindole (DAPI, Molecular Probes) to stain DNA and analyzed using a BD™ LSRII flow cytometer (Becton Dickinson) and the FlowJo™ software package.

Measurement of AR Protein Levels by Immunoblot

The indicated cells were plated in 10 cm plates in the appropriate media at 30% confluency for adherent cells or at 300,000 cells per ml for suspension cell lines. The following day suspension cells were collected by centrifugation and lysed, whereas adherent cells were lysed on the plate. Lysis was done after washing cells with PBS using lysis buffer (50 mM Tris-HCl pH 6.8, 2% SDS, 5% glycerol, 5 mM EDTA, 1 mM NaF, 10 mM β-glycerophosphate, 1 mM phenylmethylsulfonyl fluoride, 1 mM Na₃VO₄ and Roche cOmplete™ EDTA-free protease inhibitors and PhosSTOP™ phosphatase inhibitors). The media and PBS wash were reserved, centrifuged and any cells present were combined with the lysate to prevent loss of loose or unattached cells. After sonication and protein concentration normalization, 6× sodium dodecyl sulfate-polyacrylamide gel electrophoresis (SDS-PAGE) loading buffer (208 mM Tris-HCl pH 6.8, 42% glycerol, 3 M β-mercaptoethanol, 10% SDS, 5 mg/ml bromophenol blue) was added and lysates were boiled for 5 minutes. Following SDS-PAGE, immunoblots were blocked with Odyssey™ blocking buffer (LiCOR Biosciences) and incubated with primary antibody overnight at 4° C. and then secondary antibody for 1 hour at room temperature. Primary antibodies include anti-AR (N-20, Santa Cruz Biotechnology) and anti-β-actin (clone AC-15, Millipore-Sigma). Antibodies were diluted 1:1 PBS: Odyssey™ blocking buffer with 0.1% Tween-20. Immunoblots were scanned on an Odyssey™ CLx scanner (LiCOR Biosciences).

In Vivo Studies Using a Tumor-Implantable Microwell Device

C4-2 CRPC and SK-OV-3 ovarian cancer xenograft tumors were grown in four- to six-week old NCR nude mice (Taconic), male or female, respectively. Five million C4-2 or two million SK-OV-3 cells in serum free media were mixed one to one with growth factor reduced MATRIGEL® (Invitrogen) in a total volume of 200 μl and injected in the hind flank. Tumors took four to eight weeks to grow. Cylindrical microdevices (4 mm×820 μm) micromachined from medical-grade DELRIN® acetal resin blocks (DuPont) each contain eighteen 200 μm (diameter)×250 μm (depth) reservoirs. Drugs were mixed with PEG 1450 at 12.5% by weight, and 1 μg of the dry powder mixture then packed into a reservoir. Wells containing the combination of drugs contained 12.5% of each drug by weight. Tumors were excised 24 to 36 hours after device implantation, fixed in 10% formalin for 24 hours, and embedded in paraffin. Sections were stained with cleaved caspase-3 antibody followed by detection with horseradish peroxidase conjugated secondary antibody and diaminobenzidine with hematoxylin used as a counterstain, following standard immunohistochemistry techniques. Images were viewed using an EVOS® Cell Imaging System (Invitrogen) microscope, and scored in a blinded manner. The apoptotic index was calculated as the percentage of cells that stained positive for cleaved caspase-3 within a 400 μm radius of the microwell-tissue interface.

Time-Lapse Live-Cell Microscopy and Spindle Orientation Analysis

C4-2 CRPC cells expressing histone H2B-mCherry and mEmerald-tubulin. were plated in 12-well plates in phenol red free media, the following day drugs were added as indicated and the cells were imaged on a EVOS™ FL Auto Cell Imaging System equipped with an onstage incubator to maintain 37° C., adequate humidity, and 5% CO₂. Images were acquired at 15-minute intervals for 72 hours using a 20× objective lens. For the rotation analysis, the angle of the spindles major axis was tracked starting from when it was first apparent, prometaphase, until telophase. The net angular displacement is the total change in angle of the spindle during mitosis from start until finish and is the absolute value of the sum of all stepwise rotations. The cumulative angular distance is the total amount of rotation regardless of direction that occurred during mitosis and is the sum of the absolute value of all rotations.

### Results

AR-Independent Synergy Between Abiraterone and PLK1i

PLK1 is a serine/threonine kinase that plays a role in the regulation of nearly every step of mitosis and has been found to be overexpressed and associated with poor prognosis in a litany of cancers including prostate cancer. PLK1 is thought to be preferentially required for cancer cell division due to elevated replicative stress and chromosomal instability in cancer cells. Combined treatment of castrate-resistant prostate cancer (CRPC) cells with PLK inhibitors (e.g., BI2536, BI6727 (volasertib), GSK461364, and onvansertib) and abiraterone demonstrated more than additive (e.g., synergistic) tumor cell killing (FIGS. 1A-1E). Greater than additive effects from the combination of PLK inhibitor and abiraterone occurred regardless of the presence or absence of exogenous androgens through growth in media containing non-stripped FBS or charcoal stripped FBS, respectively (FIGS. 1C-1E). All other experiments were performed using media containing non-stripped FBS unless stated otherwise.

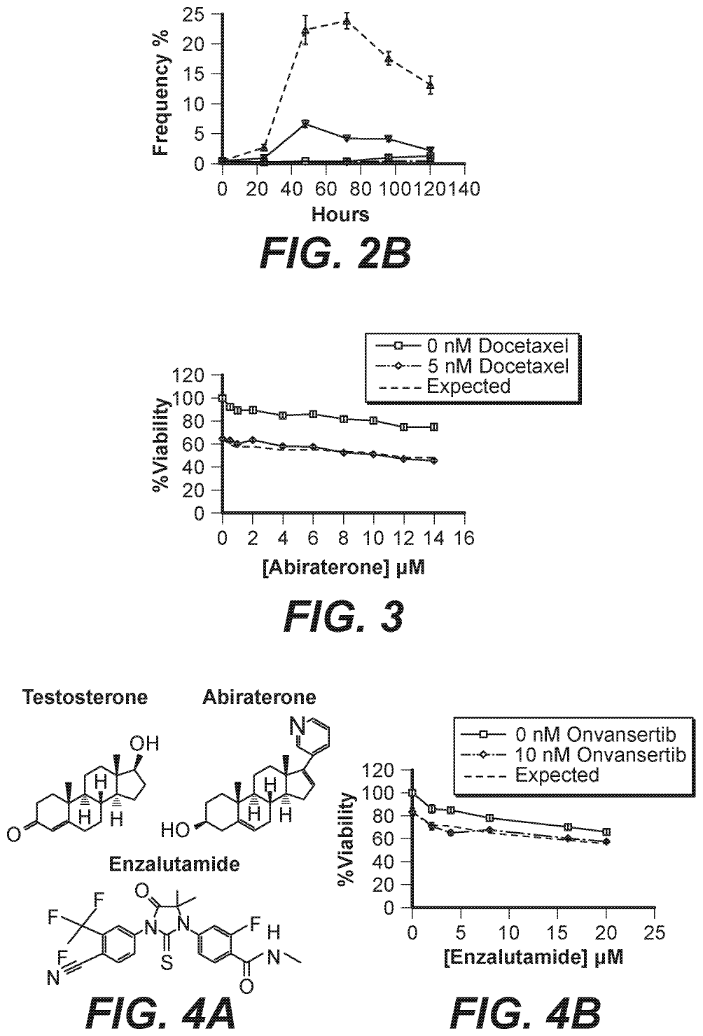

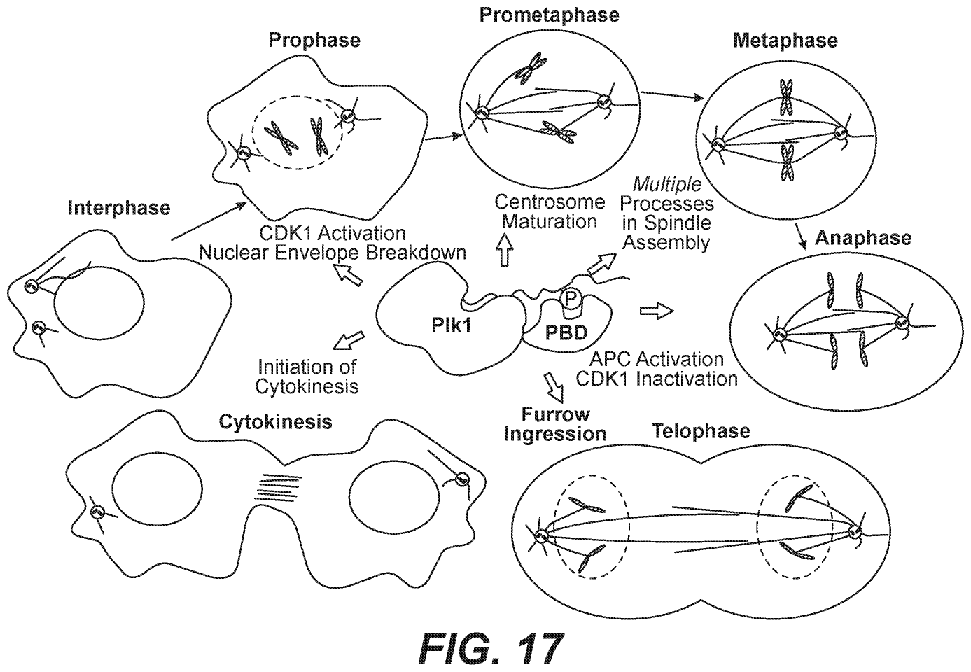

As an important regulator of mitosis in cancer cells (see FIG. 17), PLK1 inhibition causes disruption of cell division and mitotic arrest. Abiraterone treatment exacerbated the mitotic arrest caused by inhibition of PLK1, a situation that was followed by apoptotic cancer cell death (FIGS. 2A-2B). More than additive effects (e.g., synergy) was not observed when the same cell types were treated with docetaxel, an inhibitor of microtubule depolymerization and hence mitosis, indicating that the more than additive effect was not solely due to mitotic arrest caused by PLK1 inhibition (FIG. 3). Thus, the more than additive effect is specific to PLK1i and not a generic consequence of disrupting mitotic progression, as other antimitotic agents including docetaxel do not synergize with abiraterone.

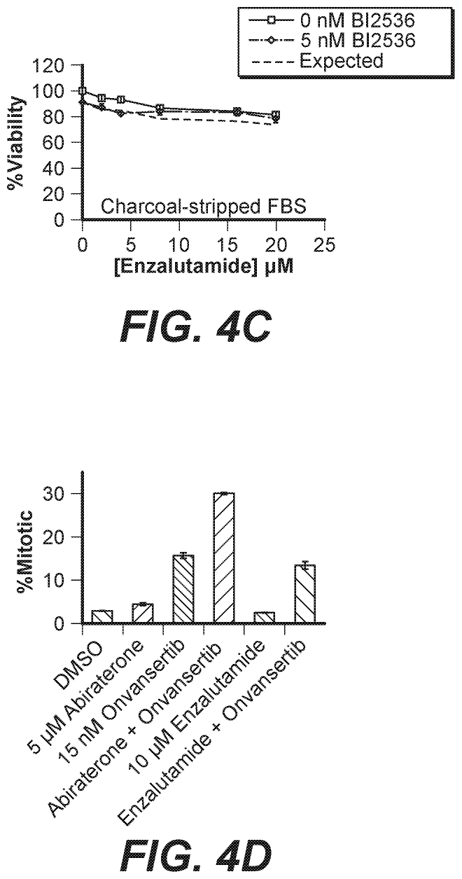

Nor is the more than additive effect likely to be due to direct AR inhibition by the metabolites of abiraterone (see FIGS. 12 and 13) as a more than additive effect was not observed between PLK1 inhibitors and the AR antagonist enzalutamide (FIGS. 4A-4C). The more than additive effect was observed in a subset of non-prostate cancer cells that lack AR expression. Thus, the more than additive effect is independent of AR activity.

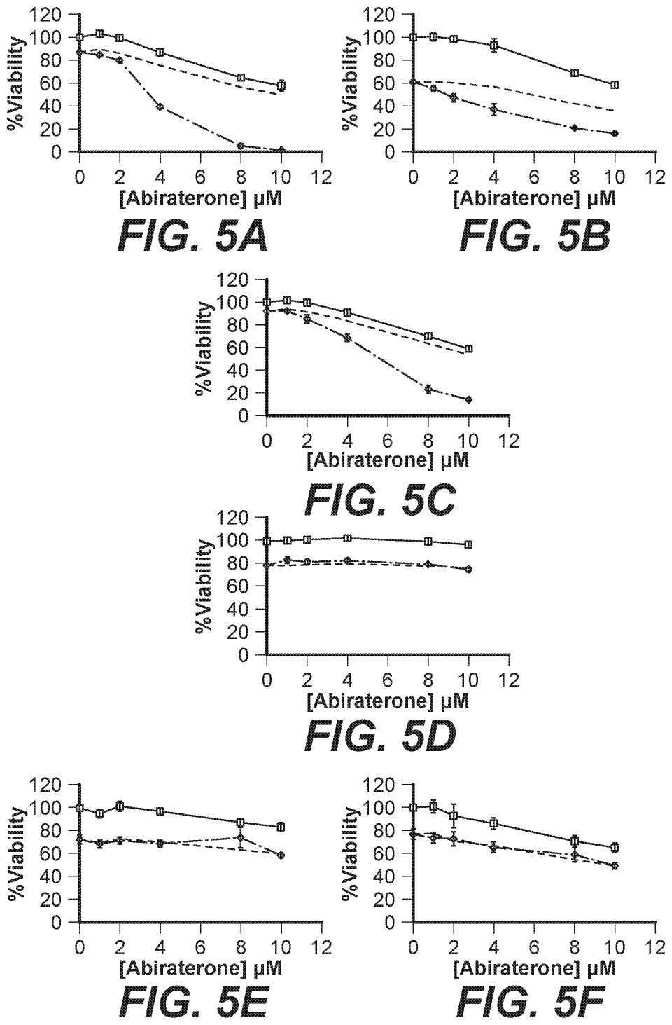

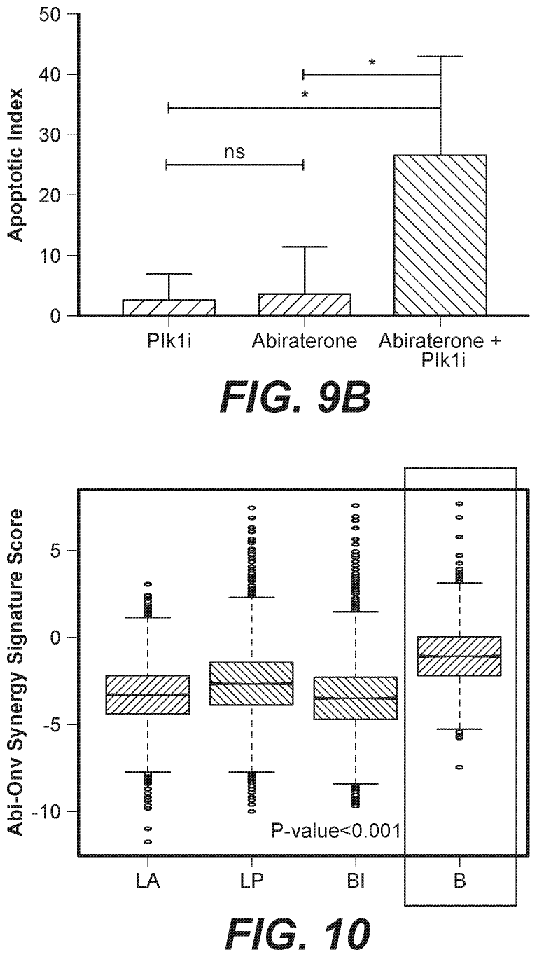

The more than additive effect between onvansertib and abiraterone is also not restricted to CRPC cells (FIGS. 5A-5F). The effect was confirmed in vivo using a drug-delivering tumor-implantable device in both C4-2 CPRC and AR-negative non-prostate cancer cell line xenografts (a SK-OV-3 ovarian cancer xenograft model). Upon implantation, drug or drug combination concentration gradients were formed. Immunohistochemistry staining for cleaved caspase-3 was used as a read out for apoptotic cell death in fields of cells adjacent to wells containing the indicated drugs and it was observed that the combination of abiraterone and PLK1i yielded increased apoptosis (see FIGS. 9A and 9B which show apoptosis indices for PLKi, abiraterone, abiraterone and PLKi treated C4-2 CRPC xenografts (FIG. 9A) or SK-OV-3 ovarian cancer xenografts (FIG. 9B)). The more than additive effect has also been demonstrated across multiple cancer types (including prostate, non-small cell lung cancer, head and neck, breast, pancreatic, ovarian, colorectal, and acute myeloid leukemia) being detected in approximately 30% of more than 90 cell lines screened (data not shown). The majority of these cell lines do not express AR. All of the non-prostate cancer cells shown in FIGS. 5A to 5F lack detectable AR protein as assayed by immunoblot. Altogether, these results indicate that the combined treatment with PLK inhibitors and abiraterone cause a more than additive (e.g., synergistic) cancer cell killing effect that is independent of AR-signaling.

Abiraterone Impairs Spindle Assembly Sensitizing Cancer Cells to PLK1i

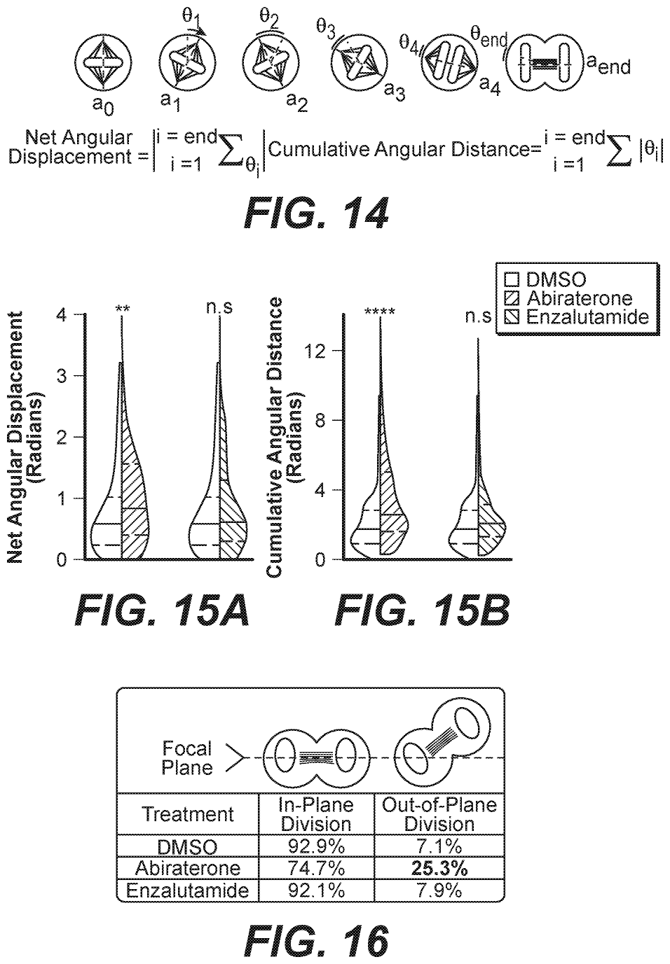

C4-2 CRPC cells that express red fluorescence protein tagged histones and green fluorescence protein tagged tubulin were analyzed using live-cell microscopy. It was found that abiraterone treatment disrupted proper spindle orientation, increasing the amount of spindle rotation during mitosis both whether defined by the difference in spindle orientation between prometaphase and telophase (net displacement) as well as the cumulative rotation throughout mitosis (FIGS. 14 and 15A-15B). Abiraterone also increased the frequency of division that was not parallel to the growth surface (FIG. 16), an observation consistent with impaired spindle orientation. Moreover, treatment with abiraterone dramatically increased the frequency of mitotic arrest caused by PLK1i (FIG. 4D). The observation that enzalutamide did not result in these phenotypes (see e.g., FIGS. 4D, and 14-16) indicates that abiraterone exerts effects on cancer cells that disrupt normal progression though mitosis that are independent of its impacts on AR signaling.

### Example 2

### Molecular Mechanism for the More than Additive Effect of Combined Abiraterone and Onvansertib Treatment Involves Upregulation of Mitosis/Meiosis Related Genes

### Materials and Methods

Experimental Design

Complementary pairs of cell lines were examined in order to unravel the biological basis of the observed more than additive effect between the PLK1 inhibitor, onvansertib, and the CYP17A1 inhibitor, abiraterone. First, two prostate cancer derived model cell lines were used: LNCaP and C4-2. LNCaP cells derive from a metastatic lesion of prostate cancer and are androgen sensitive and express prostate specific androgen (PSA). C4-2 cells were originally derived from LNCaP cells by implanting and growing LNCaP cells in castrated mice and are castrate resistant. Second, additional pairs of cell lines were examined, each pair coming from a different cancer indication, with one cell line in the pair being a responder (exhibiting a more than additive effect) and one being a non-responder (not exhibiting a more than additive effect), as previously determined from combinatorial drug screening experiments. The cell line pairs were representative of the following seven cancer indications: Prostate (PRAD), acute myeloid leukemia (AML), ovarian cancer (OV), head and neck squamous cell carcinoma (HNSCC), Her2 positive breast cancer (Her2+ BRCA), pancreatic adenocarcinoma (PAAD), and non-small cell lung cancer (NSCLC). The individual cell lines are outlined in Table 3.

TABLE 3
Cancer indications and cell line pairs examined.

| | Synergistic | Non-Synergistic | Data | |
|---|---|---|---|---|
| Indication | Cell Lines | Cell Lines | sets | |
| Prostate (CRPC) | C4-2 | LNCaP | A | C |
| Acute Myeloid Leukemia | MV-4-11 | OCI-AML-3 | B | |
| (AML) | | | | |
| Ovarian (OV) | SK-OV-3 | OAW-28 | | |
| Head/Neck(HNSCC) | CAL-33 | KY-SE-450 | | |
| Breast: HER2+ (BRCA) | AU-565 | HCC-1954 | | |
| Pancreatic (PAAD) | PSN-1 | PANC-1005 | | |
| Lung (NSCLC) | NCIH-661 | CA-LU-3 | | |

The prostate cancer cell lines were grown and treated in triplicate, yielding three biological replicates for each treatment and each of the two cell lines: C4-2 (responder) and LNCaP (non-responder). The remaining non-prostate cell lines were grown and treated singly.

As shown in Table 3, the data analyses utilized three effective data sets: A. Direct comparisons of the three biological replicates each of C4-2 and LNCaP, for each treatment; B. Pairwise comparisons among the remaining 6 non-prostate cell line pairs; and C. Pairwise comparisons among all 7 cell line pairs, after reducing the prostate replicates to a single sample to limit over-representation from the prostate samples.

Measurement of Gene Expression by RNA Sequencing

Cells were plated in 6-well plates (300,000 cells per well), and 24 hours later treated with the indicated drugs in biological triplicate. After 16 hours, cells were lysed and total RNA was isolated using NucleoSpin® RNA Plus Mini Kit (Macherey-Nagel) according to the manufacturer's recommendations. Samples were submitted to the Massachusetts Institute of Technology BioMicro Center for library preparation and sequencing. RNA quality was assessed using a Fragment Analyzer (Agilent Technologies) and RNA sequencing libraries were prepared using 400 ng of total RNA using the Kapa mRNA Hyperprep kit (Roche) at ⅓rd reaction volume using 14 cycles of PCR. Libraries were analyzed using the Fragment Analyzer and quantified by qPCR prior to pooling and sequencing on a NextSeq500 (Illumina) using 75nt single end reads.

Identification of Gene Expression Signatures

Rather than first analyzing the samples for differential gene expression, followed by ranking, and subsequent gene set enrichment analysis (GSEA, Subramanian, et al., Proc. Natl. Acad. Sci. U.S.A. 102(43): 15545-15550 (2005)), the gene by sample matrix was directly transformed to a gene set by sample matrix using gene set variation analysis (GSVA, Hänzelmann, et al., BMC Bioinformatics. 14: 7 (2013)). This results in a normally distributed ‘enrichment score’ (E.S.) for each gene set and each sample, and permits an open-ended and biologically meaningful pathway centric analyses. Prior to running GSVA, the RNAseq data was converted to gene counts, and normalized for gene length, GC content, and library depth (CQN, Hansen, et al., Biostatistics. 13(2):204-216 (2012); DESeq2, Love, et al., Genome Biol. 15: 550 (2014)). For data set C (all cell lines), the gene counts for the triplicate samples for each C4-2 and LNCaP treatment were summed, prior to library normalization, reducing each triplicate sample to one while maintaining the variance structure.

GSVA enrichment scores were calculated for 14,622 gene sets derived from the following gene set collections: MSigDB Hallmark, MSigDB Canonical Pathways, MSigDB Cancer Gene Neighborhoods, MSigDB TF Targets, MSigDB miR targets, MSigDB GO Biological Processes, MSigDB GO Molecular Function, MSigDB GO Cellular Compartments (all: Liberzon, et al., Bioinformatics. 27(12): 1739-1740 (2011); Liberzon, et al., Cell Systems. 1(6): 417-425 (2015)), and NURSA Protein Complexes (Malovannaya, et al., Cell. 145(5):787-799 (2011).

Differential enrichment (E.S. difference) was assessed using linear models as implemented by Limma (Ritchie, et al., Nucleic Acids Res. 43(7): e47 (2015)), with critical adjusted P-value of 0.001. Differential enrichment was evaluated for each treatment relative to DMSO as outlined in the contrasts in Table 4.

TABLE 4
Statistical contrasts.

| Synergistic Cell Lines | Non-Synergistic Cell Lines |
|---|---|
| Drug A vs DMSO | Drug A vs DMSO |
| Drug B vs DMSO | Drug B vs DMSO |
| Drug A & Drug B vs DMSO | Drug A & Drug B vs DMSO |

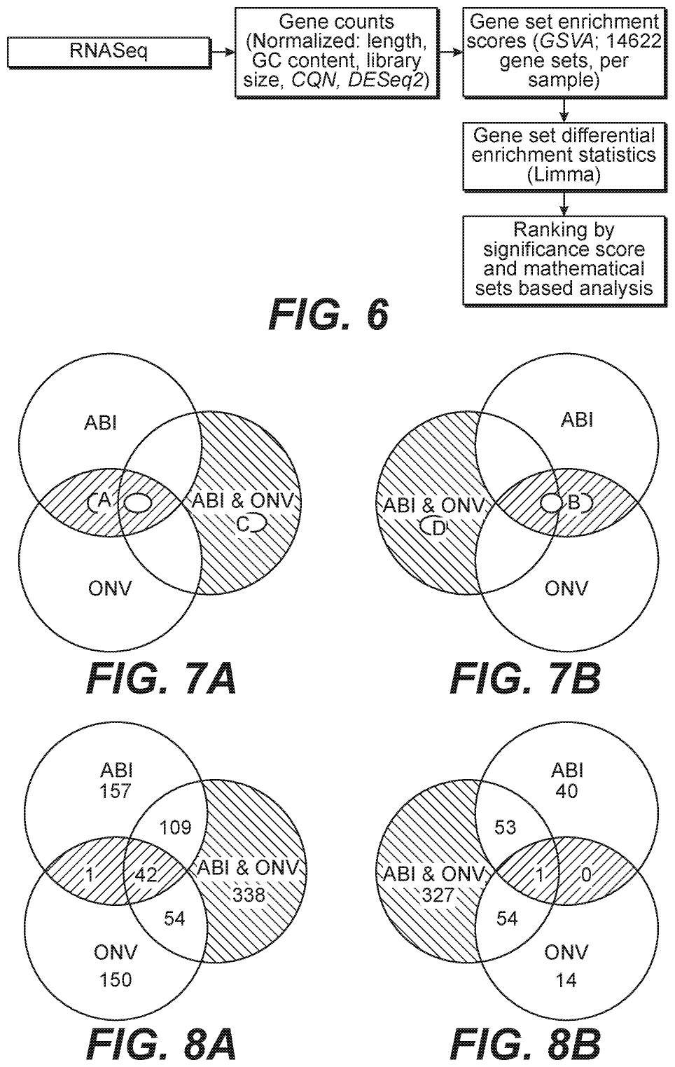

In each case, each gene set was assigned a significance score (Xiao, et al., Bioinformatics 30(6):801-807 (2014)) equal to the absolute value of the E.S. difference (analogous to log 2 fold change) multiplied by −log 10 (P.value), and ranked by decreasing significance score. The analysis schema is outlined in FIG. 6. The resulting statistics were then evaluated using a mathematical set-based strategy (FIGS. 7A-7B).

### Results

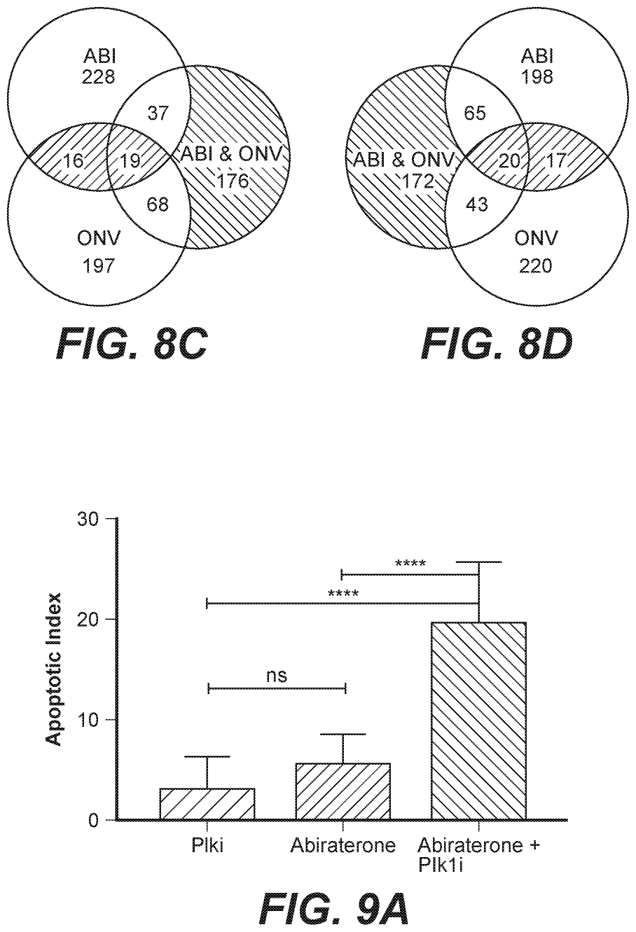

Analysis of gene expression (RNA-Seq) was used to identify groups of gene sets that represent key biological pathways and processes that are associated with the more than additive effect between onvansertib and abiraterone. For data set A, the direct comparisons of the three biological replicates each of C4-2 and LNCaP, for each treatment, 309 gene sets were identified as being significantly differentially enriched in C4-2 cells treated with abiraterone, and 247 gene sets were identified as being significantly differentially enriched in C4-2 cells treated with onvansertib (FIGS. 7A-7B, Group 1; FIG. 8A). Of these, 43 were shared between the two treatments. The same comparison in LNCaP cells revealed only one common gene set between these two treatments (FIGS. 7A-7B, Group 2; FIG. 8B). This gene set was not among the gene sets common to abiraterone and onvansertib treatments in C4-2 cells.

The presence of a common regulated group of gene sets between abiraterone treated and onvansertib treated responder C4-2 cells was not expected, as the principal mode of action of the two drugs should be quite independent—inhibition of androgen synthesis with abiraterone, and inhibition of PLK1 cell cycle related processes with onvansertib. The apparent absence of an equivalent common group of gene sets in the non-responder LNCaP cell line, indicates that this common group of gene sets is related to the observed more than additive effect in C4-2 cells.

In order to investigate the extent to which a common gene set, shared between abiraterone and onvansertib treatments, may also be indicative of a more than additive response in other, non-prostate cell lines, the equivalent comparisons were made for data set B: comparisons among the remaining 6 non-prostate cell line pairs; with no biological replicates and a paired analysis accounting for differences between indications (FIGS. 7A-7B, Groups 3 and 4; FIGS. 8C-8D). Statistical power was lower for these more complex data; therefore, the results were ranked by significance score and the top 300 gene sets compared among the treatments. In this case, 35 gene sets were common to the abiraterone and onvansertib treatments in the responder cell lines that exhibit a more than additive effect (FIGS. 7A-7B, Group 3; FIG. 8C), and 37 gene sets were common to these treatments in the non-responder cell lines (FIGS. 7A-7B, Group 4; FIG. 8D). Again, there was no overlap between these two groups of gene sets.

In order to assemble the most comprehensive set of gene sets that may be related to the more than additive effect between abiraterone and onvansertib, four groups of gene sets were gathered each from datasets A and B (see Table 3). These groups are outlined in FIGS. 7A and 7B and include:
I. Differentially enriched gene sets common to treatment with abiraterone and onvansertib in synergistic cell lines (FIG. 7A, shaded):
   ABI_{Syn}∪ONV_{Syn}
   [dataset A=43, dataset B=35]
II. Differentially enriched gene sets common to treatment with abiraterone and onvansertib in non-synergistic cell lines (B, shaded):
   ABI_{NonSyn}∪ONV_{NonSyn}
   [dataset A=1, dataset B=37]
III. Differentially enriched gene sets unique to the abiraterone plus onvansertib combination in synergistic cell lines (diagonally shaded area of C) and not differentially enriched in non-synergistic cell line contrast.
   (ABI+ONV_{Syn}/ABI_{Syn}∪ONV_{Syn})/(ABI+ONV_{NonSyn}∪(ABI_{NonSyn}∪ONV_{NonSyn}))
   [dataset A=223, dataset B=113]
IV. Differentially enriched gene sets unique to the abiraterone plus onvansertib combination in non-synergistic cell lines (diagonally shaded area of D) and not differentially enriched in synergistic cell line contrast:
   (ABI+ONV_{NonSyn}/ABI_{NonSyn}∪ONV_{NonSyn})/(ABI+ONV_{Syn}∪(ABI_{Syn}∪ONV_{Syn}))
   [dataset A=159, dataset B=135]
      This resulted in total group of 678 gene sets potentially related to a more than additive effect.

Next, the synergy scores were gathered for each of these 678 gene sets from each treatment in dataset A (CRPC C4-2 and LNCaP) and dataset B (all non-prostate cell lines). The scores were quantile normalized to bring them all to the same scale and distribution such that one or another treatment or dataset would not dominate. The data sets were then ranked by the mean normalized synergy score for abiraterone and onvansertib single agent treatments across both datasets. The inflection point of these ranked scores was calculated using the unit invariant knee method (Christopoulos D T., SSRN., (2016); available on the web at dx.doi.org/10.2139/ssrn.3043076) and 86 gene sets with significance score greater than the inflection value were kept.

Initial hierarchical clustering by gene set, and heatmap plotting of E.S. difference following onvansertib and abiraterone treatment (relative to DMSO), across all cell lines (dataset C) further refined the group of gene sets from 86 to 51 gene sets. These gene sets exhibit a clear pattern of enrichment following onvansertib treatment in both responder and non-responder cell lines. Abiraterone treatment led to a similar pattern of enrichment in these gene sets in responder cell lines, but this enrichment was absent in the non-responder cell lines. The 51 gene sets were plotted separately in a heatmap. The majority of these gene sets are directly involved in proliferation: mitotic spindle assembly and spindle checkpoint, G2M checkpoint, E2F target genes, and meiosis. Particularly striking was the differential enrichment of GO_FEMALE_MEIOSIS_CHROMOSOME_SEGREGATION and GO_PRONUCLEUS, both processes that involve organization of microtubules and spindle formation that differ significantly from the normal somatic mitotic process.

The 51 gene sets were plotted for C4-2 and LNCaP cell lines treated with the single agent treatments of abiraterone (ABI), onvansertib (ONV), enzalutamide (ENZ), and docetaxel (DTX). Neither enzalutamide nor docetaxel exhibit the shared pattern of enrichment shown by abiraterone and onvansertib in C4-2 cells. Enzalutamide is a direct antagonist of the androgen receptor (AR), a primary driver of metastatic prostate cancer. Although abiraterone primarily blocks CYP17A1 and hence, androgen synthesis, it also has a significant, albeit less potent antagonistic effect on the AR. The relative lack of response by the 51 spindle and cell division related gene sets to enzalutamide indicates that the more than additive (e.g., synergistic) killing observed with the abiraterone and onvansertib combination is independent of AR signaling. Docetaxel binds to microtubules, prevents depolymerization and inhibits mitotic spindle assembly; the lack of response by C4-2 cells to docetaxel is therefore informative regarding the aspects of the mitotic machinery and spindle that are affected by abiraterone.

The MSigDB Hallmark gene set (Liberzon et al., 2011; 2015) is a highly curated, refined collection of gene sets derived from numerous founder gene sets. The 50 Hallmark gene sets are designed to display a coherent pattern of gene expression and reflect a specific biological states or processes. A heatmap of the Hallmark gene sets across all 7 pairs of responder and non-responder cell lines (dataset C), including abiraterone (ABI), onvansertib (ONV), and the abiraterone plus onvansertib (ABI+ONV) combined treatments was plotted. Clustering identified the common upregulation of SPERMATOGENESIS, MITOTIC_SPINDLE, E2F_TARGETS, and G2M_TARGETS gene sets in responder cell lines treated with either abiraterone or onvansertib. Non-responder cell lines did not share this response to abiraterone.

In summary, the experiments described above demonstrated that castrate resistant C4-2 CRPC cells exhibit a more than additive response to treatment with the PLK1 inhibitor onvansertib and the CYP17A1 inhibitor abiraterone. This was not observed in the androgen dependent LNCaP cell line (FIG. 1F). Furthermore, there was a lack of a greater than additive response in C4-2 cells when treated with the AR inhibitor enzalutamide plus onvansertib; indicating that the more than additive effect does not result from AR-signaling (FIG. 4C-4D). In addition, a lack of a more than additive response in C4-2 cells when treated with the mitotic inhibitor docetaxel plus abiraterone demonstrated that the more than additive effect does not result solely from PLK1 inhibition leading to mitotic arrest (FIG. 3). Furthermore, as shown in FIGS. 5A-5F, the observed more than additive effect between abiraterone and onvansertib extends to multiple cancer indications (most of which are unlikely to be dependent on androgen signaling).

The experiments also allowed for the identification of a group of gene sets that are differentially enriched by responder cells (which exhibit a more than additive effect) in response to both onvansertib and abiraterone treatment. These same gene sets are not enriched in response to abiraterone treatment in non-responder cells (which do not exhibit a more than additive effect). This result was initially observed in prostate cancer cell lines, but was extended and demonstrated to apply across multiple cancer indications.

The results identified a number of gene sets that show a common enrichment in response to both abiraterone and onvansertib monotherapy treatment in CRPC C4-2 cells, which are known to respond in a more than additive way to the combination of onvansertib plus abiraterone. Although the same gene sets show a similar response to onvansertib treatment in non-responder LNCaP cells, these gene sets did not respond similarly to abiraterone treatment. The same broad group of gene sets responds similarly in responder cell lines from a broad range of cancer indications. These gene sets are upregulated by abiraterone treatment in cell lines that show a more than additive effect, but are unchanged in those that do not. This indicates a universal mechanism of abiraterone and onvansertib synergy in susceptible cancer cell lines. The more than additive effect is primarily a result of differential response by cell lines to abiraterone, and not to onvansertib. The differential response to abiraterone affects aspects of pathways and processes that onvansertib also affects. Hence, the response to abiraterone may prime the response to onvansertib in responder cell lines.

The differential response to abiraterone (its effect on gene sets that are also affected by onvansertib) indicates an AR-independent effect of abiraterone in cells that show a more than additive effect in their response to the combination. Gene sets that are shared/common to the response to abiraterone and onvansertib in responder cell lines represent pathways and processes involving mitosis, meiosis, and in particular, aspects relating to spindle formation, chromosome alignment to the spindle, spindle checkpoint, microtubule events associated with cytokinesis, mitotic-G2/M checkpoint and E2F (proliferations) associated genes. It is contemplated that both onvansertib and abiraterone may be affecting proteins and/or genes in these pathways and processes either directly, or indirectly, or both.

### Example 3

### Gene Signature for the Onvansertib-Abiraterone Response

Materials and Methods

It was observed that the C4-2 castrate resistant prostate cancer (CRPC) cell line exhibited a synergistic (SYN) response when treated with the combination of onvansertib (PLK1 inhibitor) plus abiraterone (Zytiga; CYP17A1 inhibitor). No such synergy (NON-SYN) was observed with the LNCaP CRPC cell line.

Abiraterone inhibits androgen synthesis, and also blocks the AR receptor. Enzalutamide specifically blocks the AR receptor. No synergy was observed in cell lines treated with enzalutamide+onvansertib, suggesting that the synergy is independent of AR signaling.

C4-2 cells and LNCaP cells were grown for 16 hours, in triplicate in the presence of vehicle (DMSO), abiraterone, onvansertib, the combination of abiraterone+onvansertib, enzalutamide, or the combination of enzalutamide+onvansertib.

The cell lines cultures were then subject to RNASeq.

The gene signatures described in this example relate to C4-2 and LNCaP.

RNAseq reads were quantified as gene counts using the Salmon algorithm (Patro, R. et al. Nature Methods, 14(4):417-419 (2017)) and library size normalized. The data were analyzed at two levels: (1) differential expression using DESeq2; (2) gene counts were transformed to GSVA scores (representing ˜15,000 gene sets) and differential enrichment statistics calculated using limma.

Sets based analysis identified 73 gene sets (Table 8A) that were significantly differentially enriched in common among C4-2 cells treated with abiraterone, onvansertib, or the combination abiraterone+onvansertib (all relative to vehicle). These gene sets were not differentially enriched in LNCaP cells treated with abiraterone, onvansertib, or the combination abiraterone+onvansertib. None of these gene sets were associated with enzalutamide treatment in either cell line. These “synergy gene sets” were mitosis-related and revealed a novel off-target effect of abiraterone.

The genes behind these 73 “synergy gene sets” were identified and subject to a similar set-based analysis as had been performed on the gene sets. Three synergy related groups of genes were identified from which to mine a signature:

All genes in the 73 gene sets: 1952 genes

Genes showing significant differential expression in C4-2 cells after treatment with abiraterone, but not in LNCaP cells treated with abiraterone, and in neither cell line treated with enzalutamide: (the subset of 60 genes of the 1952 genes from the 73 “synergy gene sets” that are significantly associated with abiraterone). These 60 genes are given in Table 8C.

“Synergy genes”; genes that were significantly differentially expressed in common among C4-2 cells treated with abiraterone, onvansertib, or the combination abiraterone+onvansertib (all relative to vehicle). These gene sets were not differentially enriched in LNCaP cells treated with abiraterone, onvansertib, or the combination abiraterone+onvansertib and were therefore exclusively indicative of synergy. None of these gene sets were associated with enzalutamide treatment in either cell line (15 genes, Table 7A).

As shown herein, the “All Genes” signature differentiates C4-2 and LNCaP prior to treatment, and also reflects differences between these two cell lines beyond synergy. The “Synergy Genes” signature is specific to the greater-than-additive response.

### Derivation of Gene Signatures

The above groups of genes were defined by response to treatment. It was found that many of the synergy related gene sets and genes were also differentially enriched/expressed at baseline (C4-2 [SYN] vehicle vs LNCaP [NON-SYN] vehicle).

To identify patients/cells with potentially synergistic response from baseline (pre-treatment) and previously treated with abiraterone, the following were done:

The gene signature was based on a gene weighting averaged across vehicle (DMSO) treated cell lines, and the same cell lines post-treatment with abiraterone:
A: SYN DMSO vs NON-SYN DMSO
B: SYN ABI vs NON-SYN ABI

Weight=Average(SYNᵢ)/Average (Non_SYNᵢ)

In other words, for each gene, the average gene count in C4-2 cells (across three replicates) was divided by the average gene count in LNCaP cells (across three replicates). A weight was calculated for 1) vehicle (DMSO) and 2) abiraterone treated cells, and the two weights averaged. Raw gene counts (library size corrected) were used without further transformation.

In each case, genes were separated into those with a positive weight (UP), and those with a negative weight (DOWN). Genes were then ranked by decreasing absolute weight and a minimum subset selected by a change-point analysis of the cumulative gene synergy score:

Gene_Synergy_Score=Σⁿᵢ₌₁(|geneᵢ×weightᵢ|)/1000

Thus, for each starting input gene group (All Genes=1952; Abiraterone Genes=60; Synergy genes=15), both an UP signature and a DOWN signature were derived. Without being limited to any particular theory, it is believed that these can be separately applied, or combined via ordination (plotting).

Below, for each input gene group, the final genes selected by change-point analysis plus their weights are shown for both the UP and DOWN groups. The inferred Gene Synergy Score for C4-2 and LNCaP, as well as the Euclidean distance between these scores are also shown as an example of how they may separate when plotted/ordinated:

Euclidean Distance=[((UP_{C4-2}−UP_{LNCaP})\hat{ }2)+((DOWN_{C4-2}−DOWN_{LNCaP})\hat{ }2)]\hat{ }0.5

Results

Gene “signatures” are shown in Tables 10A, 10B, 11A, 11B, 12A, and 12B.

TABLE 5A
ALL Genes (1952 input genes; 34 final genes): Weights

| UP regulated in C4-2 | | | DOWN regulated in C-4 | | |
|---|---|---|---|---|---|
| (SYNERGISTIC) | | | (SYNERGISTIC) | | |
| Rank | Gene | Weight | Rank | Gene | Weight |
| 1 | SLC7A5 | 36.2393 | 1 | TUBB4A | −11.5868 |
| 2 | HIST1H2BJ | 6.5800 | 2 | HIST1H2BG | −5.3316 |
| 3 | TUBA3D | 5.8237 | 3 | TUBA1A | −4.9269 |
| 4 | KLK2 | 4.0557 | 4 | TGFA | −4.0977 |
| 5 | ARHGEF26 | 3.8246 | 5 | RDH10 | −3.5612 |
| 6 | KLK3 | 3.5679 | 6 | GNAI1 | −3.2414 |
| 7 | SYDE2 | 3.4852 | 7 | MCCC2 | −3.0138 |
| 8 | KDELR3 | 3.4353 | 8 | CHMP4A | −2.9049 |
| 9 | KATNAL1 | 3.0724 | 9 | H2AFY2 | −2.5121 |
| 10 | SLC7A1 | 2.8630 | 10 | DYNLT1 | −2.5112 |
| 11 | H2AFJ | 2.8460 | 11 | ACPP | −2.4463 |
| 12 | KALRN | 2.8434 | 12 | OPTN | −2.3709 |
| | | | 13 | MEIS1 | −2.3603 |
| | | | 14 | SSPO | −2.2776 |
| | | | 15 | SLC19A2 | −2.2769 |
| | | | 16 | ARHGAP29 | −2.1336 |
| | | | 17 | NCKAP5L | −2.1128 |
| | | | 18 | OCRL | −2.1091 |
| | | | 19 | CLTC | −2.0850 |
| | | | 20 | CYFIP2 | −2.0554 |
| | | | 21 | RSPH1 | −2.0545 |
| | | | 22 | SEPT6 | −2.0452 |

TABLE 5B
ALL Genes (1952 input genes; 34 final genes):
Gene_Synergy_Score for C4-2, LNCaP

| | UP | UP | DOWN | DOWN | |
|---|---|---|---|---|---|
| | (12 Genes) | (12 Genes) | (22 Genes) | (22 Genes) | |
| | C4-2 | LNCaP | C4-2 | LNCaP | Euclidean |
| Treatment | (SYN) | (NON-SYN) | (SYN) | (NON-SYN) | Distance |
| DMSO | 378.26 | 65.34 | 37.84 | 93.64 | 317.84 |
| Abiraterone | 349.70 | 35.85 | 34.62 | 89.10 | 318.32 |
| Onvansertib | 376.90 | 67.09 | 34.63 | 90.91 | 467.81 |
| Abiraterone + | 342.14 | 41.38 | 32.11 | 86.49 | 374.25 |
| Onvansertib | | | | | |

TABLE 6A
Abiraterone Genes (60 input genes; 40 final genes): Weights

| UP regulated in C4-2 | | | DOWN regulated in C4-2 | | |
|---|---|---|---|---|---|
| (SYNERGISTIC) | | | (SYNERGISTIC) | | |
| Rank | Gene | Weight | Rank | Gene | Weight |
| 1 | AGR2 | 1.9153 | 1 | OPTN | −2.3709 |
| 2 | KNL1 | 1.6120 | 2 | CLTC | −2.0850 |
| 3 | FMNL2 | 1.5765 | 3 | GDAP1 | −1.7810 |
| 4 | PRC1 | 1.5293 | 4 | SMAD3 | −1.7277 |
| 5 | HJURP | 1.5172 | 5 | DNMT3A | −1.5190 |
| 6 | PSRC1 | 1.4531 | 6 | WFS1 | −1.4376 |
| 7 | ASPM | 1.4521 | 7 | SETDB2 | −1.4183 |
| 8 | OPA1 | 1.4509 | 8 | GDI1 | −1.2611 |
| 9 | CCNB2 | 1.4283 | 9 | AGO4 | −1.2265 |
| 10 | TAX1BP3 | 1.4053 | 10 | CUL7 | −1.2233 |
| 11 | ESCO2 | 1.3905 | 11 | ACTG1 | −1.1543 |
| 12 | PRKCD | 1.3796 | 12 | FANCD2 | −1.1335 |
| 13 | CKAP2L | 1.3264 | 13 | ITGB1 | −1.1321 |
| 14 | RACGAP1 | 1.3180 | 14 | STMN1 | −1.1302 |
| 15 | PLK4 | 1.3102 | 15 | ARHGAP12 | −1.1292 |
| 16 | DLGAP5 | 1.2769 | 16 | XPO1 | −1.1021 |
| 17 | TROAP | 1.2545 | 17 | POC1B | −1.0470 |
| 18 | DVL3 | 1.2433 | 18 | SLC19A1 | −0.0661 |
| 19 | KPNA2 | 1.2158 | | | |
| 20 | CALM1 | 1.1946 | | | |
| 21 | SELENOS | 1.1672 | | | |
| 22 | UBE2C | 1.1372 | | | |

TABLE 6B
Abiraterone Genes (60 input genes; 40 final genes):
Gene_Synergy_Score for C4-2, LNCaP

| | UP | UP | DOWN | DOWN | |
|---|---|---|---|---|---|
| | (22 Genes) | (22 Genes) | (18 Genes) | (18 Genes) | |
| | C4-2 | LNCaP | C4-2 | LNCaP | Euclidean |
| Treatment | (SYN) | (NON-SYN) | (SYN) | (NON-SYN) | Distance |
| DMSO | 55.68 | 43.07 | 67.22 | 98.40 | 33.63 |
| Abiraterone | 63.51 | 45.77 | 71.52 | 96.33 | 30.50 |
| Onvansertib | 62.33 | 49.85 | 66.95 | 99.89 | 35.22 |
| Abiraterone + | 72.51 | 54.46 | 64.02 | 98.23 | 38.67 |
| Onvansertib | | | | | |

TABLE 7A
Synergy Genes (15 input genes; 15 final genes): Weights

| UP regulated in C4-2 | | | DOWN regulated in C4-2 | | |
|---|---|---|---|---|---|
| (SYNERGISTIC) | | | (SYNERGISTIC) | | |
| Rank | Gene | Weight | Rank | Gene | Weight |
| 1 | KNL1 | 1.6120 | 1 | CLTC | −2.0850 |
| 2 | PRC1 | 1.5293 | 2 | XPO1 | −1.1021 |
| 3 | HJURP | 1.5172 | 3 | HMGB3 | −0.0255 |
| 4 | PSRC1 | 1.4531 | | | |
| 5 | ASPM | 1.4521 | | | |
| 6 | CCNB2 | 1.4283 | | | |
| 7 | CKAP2L | 1.3264 | | | |
| 8 | RACGAP1 | 1.3180 | | | |
| 9 | DLGAP5 | 1.2769 | | | |
| 10 | TROAP | 1.2545 | | | |
| 11 | KPNA2 | 1.2158 | | | |
| 12 | KIF14 | 1.0664 | | | |

TABLE 7B
Synergy Genes (15 input genes): Gene_Synergy_Score for C4-2, LNCaP

| | UP | UP | DOWN | DOWN | |
|---|---|---|---|---|---|
| | (12 Genes) | (12 Genes) | (3 Genes) | (3 Genes) | |
| | C4-2 | LNCaP | C4-2 | LNCaP | Euclidean |
| Treatment | (SYN) | (NON-SYN) | (SYN) | (NON-SYN) | Distance |
| DMSO | 32.10 | 24.60 | 24.15 | 44.84 | 21.99 |
| Abiraterone | 37.13 | 26.27 | 22.91 | 42.20 | 22.14 |
| Onvansertib | 38.67 | 31.13 | 22.22 | 44.93 | 23.93 |
| Abiraterone + | 48.14 | 35.35 | 20.88 | 43.65 | 26.12 |
| Onvansertib | | | | | |

TABLE 8A
The 73 gene sets identified by set based analysis that were significantly differentially
enriched in common among C4-2 cells treated with abiraterone, onvansertib, or
the combination abiraterone + onvansertib (all relative to vehicle).

GO_CONDENSED_CHROMOSOME_OUTER_KINETOCHORE
GO_CHROMOSOME_PASSENGER_COMPLEX
GNF2_H2AFX
GNF2_CDC20
GNF2_CCNB2
GNF2_MKI67
GNF2_BUB1
GNF2_ESPL1
GNF2_CDC2
GNF2_HMMR
GNF2_SMC2L1
GNF2_CCNA2
GNF2_TTK
GNF2_RRM2
GNF2_CENPF
PID_AURORA_B_PATHWAY
GO_CONDENSIN_COMPLEX
GO_REGULATION_OF_ATTACHMENT_OF_SPINDLE_MICROTUBULES_TO_KINETOCHORE
PID_AURORA_A_PATHWAY
GO_MICROTUBULE_PLUS_END
GO_ESTABLISHMENT_OF_MITOTIC_SPINDLE_LOCALIZATION
GO_REGULATION_OF_CHROMOSOME_SEGREGATION
GO_MITOTIC_SISTER_CHROMATID_SEGREGATION
GO_REGULATION_OF_SPINDLE_ORGANIZATION
HALLMARK_G2M_CHECKPOINT
REACTOME_MITOTIC_METAPHASE_AND_ANAPHASE
GO_SPINDLE_ORGANIZATION
GO_MITOTIC_NUCLEAR_DIVISION
GO_MITOTIC_SPINDLE
GO_NUCLEAR_CHROMOSOME_SEGREGATION
GO_CHROMOSOME_SEGREGATION
GO_CONDENSED_CHROMOSOME
GO_SPINDLE_ASSEMBLY
REACTOME_M_PHASE
GO_SPINDLE_POLE
GO_SPINDLE
GO_ORGANELLE_FISSION
GO_CHROMOSOMAL_REGION
REACTOME_SIGNALING_BY_RHO_GTPASES
REACTOME_RHO_GTPASE_EFFECTORS
GNF2_SMC4L1
GNF2_RRM1
GNF2_FEN1
GNF2_CKS1B
GO_PROTEIN_LOCALIZATION_TO_CHROMOSOME_CENTROMERIC_REGION
GNF2_PCNA
GNF2_CKS2
REACTOME_MITOTIC_SPINDLE_CHECKPOINT
REACTOME_MITOTIC_PROMETAPHASE
REACTOME_RESOLUTION_OF_SISTER_CHROMATID_COHESION
GO_CHROMOSOME_CENTROMERIC_REGION
REACTOME_RHO_GTPASES_ACTIVATE_FORMINS
GO_MITOTIC_SPINDLE_ORGANIZATION
GO_MICROTUBULE_CYTOSKELETON_ORGANIZATION_INVOLVED_IN_MITOSIS
GO_CONDENSED_CHROMOSOME_CENTROMERIC_REGION
GO_KINETOCHORE
REACTOME_ANCHORING_OF_THE_BASAL_BODY_TO_THE_PLASMA_MEMBRANE
GO_CILIARY_BASAL_BODY_PLASMA_MEMBRANE_DOCKING
REACTOME_REGULATION_OF_PLK1_ACTIVITY_AT_G2_M_TRANSITION
REACTOME_CENTROSOME_MATURATION
REACTOME_AURKA_ACTIVATION_BY_TPX2
PID_PLK1_PATHWAY
GO_ATTACHMENT_OF_SPINDLE_MICROTUBULES_TO_KINETOCHORE
GO_MITOTIC_CHROMOSOME_CONDENSATION
PID_SMAD2_3PATHWAY
GO_MCM_COMPLEX
GO_VITAMIN_METABOLIC_PROCESS
GO_CELLULAR_RESPONSE_TO_TOPOLOGICALLY_INCORRECT_PROTEIN
GO_WATER_SOLUBLE_VITAMIN_METABOLIC_PROCESS
GO_ER_NUCLEUS_SIGNALING_PATHWAY
GO_BIOTIN_METABOLIC_PROCESS
GO_EUKARYOTIC_TRANSLATION_INITIATION_FACTOR_2B_COMPLEX
GO_SISTER_CHROMATID_SEGREGATION

TABLE 8B
Genes contained in 45 of the 73 gene sets shown in Table 8A. The
genes contained in the remaining gene sets are shown in Table 2.

| | |
|---|---|
| GNF2_CKS1B | ASF1B, ASPM, ATIC, AURKB, BIRC5, BUB1, CCNA2, CCT4, CDC20, |
| | CDCA8, CENPE, CKS1B, ESPL1, GMPS, IARS, KIF11, KIF20A, KIF2C, |
| | KNTC2, MCM3, MCM6, MELK, MRPL35, NCAPD2, PA2G4, PAICS, RAN, |
| | RANBP1, RFC3, RFC4, RNASEH2A, RPA3, SMC2, TCP1, TPX2, TRIP13, |
| | UBE2C |
| GNF2_FEN1 | CCNA2, CCNB2, CDC2, CENPF, CKS2, COPS3, DHFR, DKC1, DLG7, |
| | DNAJC9, DTL, DTYMK, DUT, E2F8, FEN1, FOXM1, GINS1, GINS2, |
| | GMNN, HAT1, HMGB2, HMMR, HNRPAB, KIAA0101, KIAA1794, KIF11, |
| | KNTC2, LOC146909, MCM2, MCM3, MCM4, MCM5, MCM7, METAP2, |
| | MSH2, NASP, NOLA1, NUSAP1, PCCB, PCNA, PLK4, POLE2, PRC1, |
| | RACGAP1, RAD51AP1, RFC4, RFC5, RRM1, SHCBP1, SMC4, SNRPD1, |
| | TOP2A, TTK, TYMS, VRK1, ZWINT |
| GNF2_RRM1 | ANP32E, ASPM, AURKA, AURKB, BIRC5, BUB1B, CCNA2, CCNB2, |
| | CCT6A, CDC2, CDC20, CDCA8, CENPE, CENPF, CENPM, CHCHD3, CKS2, |
| | DHFR, DLG7, DTL, DTYMK, E2F8, EIF2S1, FEN1, FH, FOXM1, GINS1, |
| | GINS2, GMNN, H2AFX, HAT1, HMGB2, HMMR, HNRPAB, KIAA0101, |
| | KIAA1794, KIF11, KIF14, KNTC2, LOC14690, MCM2, MCM3, MCM4, |
| | MCM5, MCM6, MCM7, MELK, METAP2, MKI67, MRPL35, MSH2, MT1JP, |
| | NASP, NUP37, NUSAP1, PA2G4, PAICS, PCCB, PCNA, PLK4, PRC1, |
| | PRIM1, PSMD14, PTGES3, RACGAP1, RAD51AP1, RAN, RFC3, RFC4, |
| | RRM1, RRM2, SERBP1, SHCBP1, SLBP, SMC2, SMC4, SNRPD1, SNRPG, |
| | SSRP1, TCP1, TMPO, TOP2A, TTK, TYMS, UBE2C, USP14, VRK1, ZWINT |
| GNF2_SMC4L1 | ANP32B, ANP32E, ASPM, AURKA, BUB1B, CBX3, CCNA2, CCNB2, CDC2, |
| | CDC20, CDCA8, CENPF, CKAP2, CKS2, COPS3, DEK, DHFR, DKC1, DLG7, |
| | DNAJC9, DTL, DTYMK, DUT, E2F8, EIF2S1, EXOSC8, FBXO5, FEN1, |
| | FOXM1, GINS1, GINS2, GMNN, H2AFX, HAT1, HMGB2, HMMR, HNRPA3, |
| | HNRPAB, KIAA010, KIAA179, KIF11, KNTC2, LOC1469, MCM2, MCM3, |
| | MCM4, MCM5, MCM7, METAP2, MRPL35, MSH2, NASP, NOLA1, NUP153, |
| | NUSAP1, PCCB, PCNA, PLK4, POLE2, PRC1, PRIM1, RACGAP1, |
| | RAD51AP, RFC3, RFC4, RRM1, SERBP1, SFRS1, SHCBP1, SLBP, SMC2, |
| | SMC4, SNRPD1, SNRPG, SSRP1, SUPT16H, TMPO, TOP2A, TTK, TYMS, |
| | USP1, VRK1, ZWILCH, ZWINT |
| GO_BIOTIN_{—} | HLCS, PC, PCCA, PCCB, PDZD11, MCCC1, MCCC2, BTD, SLC5A6 |
| METABOLIC_{—} | |
| PROCESS | |
| GO_CELLULAR_{—} | HDAC6, PLA2G4, BCL2L1, CTDSP2, PREB, PDIA6, OPTN, PIGBOS, |
| RESPONSE_TO_{—} | STUB1, RACK1, CLGN, CREB3, HYOU1, AGR2, ERN2, YIF1A, PDIA5, |
| TOPOLOGICALLY_{—} | COPS5, KDELR3, FICD, KLHDC3, ADD1, TPP1, CALR3, PACRG, ATF6B, |
| INCORRECT_{—} | CREB3L, CTH, DAB2IP, CREBRF, DAXX, DCTN1, DDIT3, DDX11, |
| PROTEIN | BHLHA1, TOR1A, EIF2S1, DNAJC1, EP300, ERN1, EXTL1, EXTL2, EXTL3, |
| | SEC31A, ATF6, UFL1, PPP1R1, SDF2L1, FGF21, HSPB8, TBL2, AMFR, |
| | GFPT1, SERP1, RNF175, GSK3A, POMT2, ERO1A, HDGF, CXXC1, HSF1, |
| | HSPA1A, HSPA1B, HSPA1L, HSPA2, HSPA5, HSPA6, HSPA7, HSPA8, |
| | HSPA9, HSPD1, IGFBP1, CXCL8, ACADVL, SERP2, LMNA, DNAJB9, |
| | ATXN3, GET3, ASNS, SULT1A, ATF3, ATF4, NCK1, NFE2L2, PRKN, |
| | DERL2, HSPA14, BFAR, MBTPS2, DNAJB1, TM7SF3, PIK3R1, DNAJB1, |
| | PARP16, AUP1, FKBP14, WIPI1, ANKZF1, TMEM33, PPP2R5, UBE2W, |
| | RNF121, YOD1, RNF126, ARFGAP, UGGT2, SELENO, MYDGF, EIF2AK, |
| | DNAJC3, UGGT1, RHBDD, PTPN1, PTPN2, BAK1, BAX, SRPRB, CCND1, |
| | RNF5, CCL2, TSPYL2, HERPUD, SHC1, CREB3L, DDRGK, BOK, SRPRA, |
| | SSR1, HSPA13, SULT1A, TMBIM6, TLN1, HSP90B, UFD1, VCP, WFS1, |
| | XBP1, ZBTB17, DERL1, BAG6, DNAJB1, CDK5RA, KLHL15, CALR, |
| | CANX, RHBDD, NCK2, SYVN1, CUL3, CREB3L, PPP1R1, STC2, MBTPS1, |
| | CREB3L, ATP6V0, DERL3, RNF185, VAPB, EIF2AK, BAG3, GOSR2, |
| | EDEM1, HERPUD, TATDN2, CUL7 |
| GO_CHROMOSOMAL_{—} | TEN1, RAD50, CDK2, ALYREF, STAG1, IKZF1, SYCP2, NDC80, CFDP1, |
| REGION | CEBPB, DCTN2, CENPA, CENPB, CENPC, SPAG5, CENPE, CENPF, CTCF, |
| | DCTN6, PTGES3, STAG3, STAG2, SUGT1, CBX1, KIF2C, SOX30, CHEK1, |
| | ZWINT, KAT7, DMC1, CHEK2, PMF1, DCTN3, CDCA5, CBX3, OIP5, |
| | MBD6, ANAPC16, H4-16, NUDCD2, PARP1, DYDC1, CSNK1A1, TERB2, |
| | SPC24, KASH5, SGO2, SGO1, ESCO2, DAXX, DCTN1, DDB1, DHX36, |
| | DNA2, DYNC1I1, DYNC1LI, DNMT1, DNMT3A, NSMCE1, CENPV, |
| | CENPX, ZSCAN4, ERCC1, ERCC4, EZH1, EZH2, SKA1, SKA3, H2AC1, |
| | SYCP2L, LRWD1, FEN1, SIRT2, SCMH1, KDM1A, PDS5B, WAPL, SETX, |
| | KDM4C, CLASP2, SMC5, SEPTIN6, FBXO28, PDS5A, NUP160, FBXW11, |
| | SMG6, NCAPD3, FMR1, CLASP1, SMCHD1, ITGB3BP, CBX5, ORC3, |
| | SPO11, TFIP11, CENPI, GPATCH1, XRCC6, H2BC1, SUN2, AHCTF1, POT1, |
| | BOD1L1, NSL1, SIN3A, NGDN, SS18L1, TINF2, MTBP, SMC1B, ZNF330, |
| | MAJIN, CHAMP1, TERB1, KANSL1, BOD1L2, NSMCE2, RPA4, H2AX, |
| | H2BC3, H3-3A, H3-3B, HELLS, ZBTB48, HNRNPA, HNRNPU, APC, APEX1, |
| | HSF1, BIRC5, SKA2, NUP43, INCENP, CENPS, KIF22, CENPW, LIG4, |
| | CENPP, MAD2L1, MCM2, MCM3, MCM4, MCM5, MCM6, MCM7, MEN1, |
| | MRE11, MSH2, PPP1R12, NBN, ATM, SEPTIN2, NEK2, NUP98, EID3, |
| | ORC1, ORC2, ORC4, ORC5, PAFAH1B, SYCP3, PCNA, KMT5B, TRAPPC1, |
| | DYNC1LI, DCTN4, SPOUT1, SIRT6, RTEL1, PHF2, PKHD1, PLK1, PML, |
| | MIS18A, POLD1, POLR2B, TERF2IP, GAR1, ATR, ATRX, NSMCE4, NDE1, |
| | ERCC6L, SPDL1, PINX1, PPP1CA, PPP1CB, PPP1CC, ZWILCH, PIWIL2, |
| | WRAP53, PPP1R10, CDCA8, PPP2CA, PPP2CB, CENPQ, PPP2R1A, RIF1, |
| | NAT10, PPP2R5A, PPP2R5C, MIS18BP, ZGRF1, HJURP, H4C15, MACROH, |
| | NHP2, NLRP2, KDM4D, NUP133, MBD5, LRIF1, CENPN, PRKDC, RCC2, |
| | TEX14, NSMCE3, PSEN1, PSEN2, WRNIP1, KNL1, NUP107, PELI1, THOC2, |
| | SPC25, PURA, RAD17, RAD21, RAD51, RAD51D, RANGAP1, UPF1, RPA1, |
| | RPA2, CLIP1, SALL1, SEC13, BLM, CENPK, NCAPG, RAD21L1, DCLRE1C, |
| | MEAF6, DCLRE1B, NABP1, CENPH, ACD, SUMO3, SNAI1, SP100, SSB, |
| | BRCA2, AURKA, AURKC, SUV39H1, SYCP1, BUB1, DYNLT3, BUB1B, |
| | TEP1, TERC, TERF1, TERF2, TERT, TP53BP1, TPR, TTK, MEIKIN, |
| | UVRAG, WRN, XPO1, XRCC1, XRCC3, XRCC5, ZNF207, MIS12, CENPM, |
| | NUP37, NABP2, DSCC1, CENPO, THOC6, CDC73, HMBOX1, SMC6, |
| | CENPU, SUV39H2, NUP85, DSN1, STN1, PIF1, THOC7, CENPT, CTC1, |
| | WDR82, TNKS2, NDEL1, CDT1, SEH1L, KIF18A, SMC1A, H3-4, H4C9, |
| | NUF2, H4C1, H4C4, H4C6, H4C12, H4C11, H4C3, H4C8, H4C2, H4C5, |
| | H4C13, H4C14, MAD1L1, KAT8, PHF6, THOC3, DYDC2, DOT1L, SLX4, |
| | DCTN5, KIF2B, DPY30, KMT5C, HAT1, THOC5, DYNLL1, TNKS, KAT2B, |
| | CCNB1, RNF8, BAZ1B, KNSTRN, SMC3, BOD1, CENPL, ZW10, BUB3, |
| | AURKB, ZNF276, RECQL4, MACROH, KNTC1, RASSF2, CKAP5, CDK1, |
| | TOX4, SEPTIN7, TELO2, NCAPD2, DCLRE1A, THOC1, REC8 |
| GO_CHROMOSOME_{—} | BIRC5, INCENP, CDCA8, AURKA, AURKC, AURKB |
| PASSENGER_{—} | |
| COMPLEX | |
| GO_CHROMOSOME_{—} | SYCE1L, UBE2DN, DDX11L8, RNF212B, SMC4, MAGEA1, MAGEA5, |
| SEGREGATION | ACTR3, ACTR2, MEI4, AKAP8, STAG1, KATNB1, SYCP2, NDC80, |
| | MAD2L2, TACC3, PIBF1, DCTN2, SMC2, CENPC, P3H4, SPAG5, CENPE, |
| | CENPF, RGS14, CTCF, STAG3, STAG2, PTTG2, NEK6, KIF2C, TLK2, |
| | RCC1, TENT4A, UBE2C, ZWINT, DMC1, PMF1, CDCA5, PHB2, OIP5, |
| | ESCO1, RMI2, ARL8A, CHMP4B, DIS3L2, M1AP, CORT, BANF2, TERB2, |
| | CSNK2A, CSNK2A, KIF18B, EME1, KASH5, PHF13, CTNNB1, HORMAD, |
| | MEI1, TTL, SGO2, SGO1, SPICE1, CDCA2, ESCO2, MCMDC2, SHOC1, |
| | HFM1, DDX3X, DDX11, DYNC1H, DUSP1, ECT2, AGO4, EME2, |
| | KLHDC8B, CENPX, ERCC2, ERCC4, FANCD2, SKA1, SKA3, FEN1, |
| | MAPK15, SIRT2, PDS5B, WAPL, POGZ, SMC5, ABRAXA, RRS1, PDS5A, |
| | NCAPD3, SUN1, PUM2, MAU2, NCAPH, SPO11, NUP62, KIF4A, CDC26, |
| | MEIOB, ANKRD3, SYCE2, NIPBL, ANAPC15, NSL1, CHMP2B, PTTG3P, |
| | FBXO5, GEM, AKAP8L, MLH3, SMC1B, TUBG2, VPS4A, CHMP2A, |
| | MAJIN, CHAMP1, TERB1, MEIOC, NEK8, RNF212, KIF4B, NSMCE2, |
| | NTMT1, NR3C1, CHMP4A, RACGAP, SLC25A5, NCAPH2, ANAPC4, |
| | C14orf39, HNRNPU, APC, BIRC5, IHO1, SKA2, GEN1, NUP43, IK, INCENP, |
| | CENPS, KIFC1, KIF25, KIF22, KPNB1, CENPW, MAD2L1, MAGEA5, |
| | MKI67, MLH1, MOS, MRE11, DDX12P, STAG3L3, STAG3L2, MSH4, ATM, |
| | NEK2, NUMA1, WRAP73, TRAPPC1, RMDN1, DYNC1LI, CHMP1A, |
| | NUSAP1, ANAPC5, ANAPC7, LCMT1, CHMP5, ANAPC11, SIRT7, CIAO2B, |
| | PLK1, MIS18A, STAG3L1, ATRX, INO80, MAP10, NDE1, NCAPG2, SPDL1, |
| | CHTF8, PINX1, CDCA8, MSTO1, CENPQ, PPP2R1A, ARL8B, FBXW7, |
| | HJURP, NDC1, SLF2, CDK5RA, RIOK2, PCID2, CENPN, HDAC8, RCC2, |
| | TEX15, TEX14, TEX12, TEX11, BEX4, FMN2, RAD18, SMARCA, PRDM9, |
| | PSMG2, KNL1, CHMP1B, SPC25, HECW2, FANCM, CCNB1IP, RAD21, |
| | RAD51C, RAN, RB1, RPS3, CENPK, NCAPG, SFPQ, MMS19, RAD21L1, |
| | SYCE3, ANAPC1, CEP85, STAG3L4, CENPH, BRCA1, SRPK1, AURKC, |
| | SYCP1, BUB1, BUB1B, TERF1, TOP1, TOP2A, TOP2B, TOP3A, TPR, TTK, |
| | TTN, TUBG1, MEIKIN, HIRA, UBE2B, UBE2I, UVRAG, XRCC3, ZNF207, |
| | MIS12, NUP37, DSCC1, BAG6, CHMP6, SMC6, NEK11, MCMBP, NAA60, |
| | DSN1, ANKRD5, RMI1, CENPT, MUS81, NAA50, NDEL1, FAM83D, CDT1, |
| | SEH1L, KIF18A, USP9X, SMC1A, NAA10, AXIN2, NUF2, MAD1L1, |
| | SETDB2, HASPIN, BRIP1, HORMAD, USP44, ABRAXA, CIAO2A, SLF1, |
| | SLX4, CUL3, KIF2B, SPATA22, PSRC1, KLHL22, MAEL, TNKS, BECN1, |
| | CDC23, RAB11A, RIOK3, BANF1, CDC16, CCNB1, TOP3B, CCNE1, |
| | KNSTRN, PRC1, LATS1, SMC3, BOD1, CCNE2, CHMP7, ZW10, BUB3, |
| | AURKB, PTTG1, CHMP4C, TRIP13, SYCE1, CIAO1, RECQL5, KIF23, |
| | VPS4B, MACROH, PUM1, ESPL1, DLGAP5, TLK1, CDC6, CDC20, |
| | NCAPD2, KIF14, CDC27, CDC42, REC8 |
| GO_CILIARY_{—} | ACTR1A, AKAP9, TUBB4A, TUBB4B, DCTN2, CETN2, PLK4, SDCCAG, |
| BASAL_BODY_{—} | CNTRL, CEP43, CEP250, DCTN3, HAUS1, RAB3IP, NEDD1, SCLT1, |
| PLASMA_{—} | CSNK1D, CSNK1E, TTBK2, DCTN1, DYNC1H, DYNC1I2, TUBB, CEP162, |
| MEMBRANE_{—} | CEP164, MAPRE1, NINL, CEP131, CEP152, RPGRIP1, CLASP1, HAUS5, |
| DOCKING | C2CD3, TCTN3, NPHP4, B9D1, HSP90AA, KIF24, NEK2, NPHP1, ODF2, |
| | PAFAH1B, PCM1, CEP83, PCNT, TMEM21, PLK1, HAUS6, AHI1, NDE1, |
| | MKS1, HAUS4, CEP192, HAUS2, PPP2R1A, HAUS7, PRKACA, CEP72, |
| | CDK5RAP, PRKAR2B, CENPJ, CC2D2A, MARK4, TUBA4A, TUBG1, |
| | YWHAE, YWHAG, ALMS1, TUBA1A, HAUS3, CEP97, TCTN1, TCTN2, |
| | CEP76, CEP290, CEP63, CEP70, B9D2, CEP78, OFD1, CEP89, FBF1, SSNA1, |
| | DYNLL1, RAB11A, TMEM67, HAUS8, CEP41, IQCB1, CEP135, CEP57, |
| | CCP110, CKAP5, SFI1, CDK1 |
| GO_CONDENSED_{—} | SYCE1L, RNF212B, SMC4, RAD50, LRPPRC, CDK2, MEI4, AKAP8, STAG1, |
| CHROMOSOME | SYCP2, NDC80, CFDP1, CDX2, CEBPB, CENPA, CENPB, SMC2, CENPC, |
| | P3H4, SPAG5, CENPE, CENPF, CTCF, DCTN6, STAG3, STAG2, KIF2C, |
| | RCC1, TOPBP1, CHEK1, ZWINT, DMC1, PMF1, DCTN3, CBX3, ANAPC16, |
| | ADD3, NUDCD2, HUS1B, RAD9B, CSNK1A1, SPC24, KASH5, HORMAD, |
| | SGO2, SGO1, SHOC1, DDX11, FAM9A, FAM9B, FAM9C, DYNC1I1, AGO3, |
| | NSMCE1, CENPV, CENPX, ESRRB, FANCD2, SKA1, SKA3, SYCP2L, |
| | LRWD1, KIFAP3, WAPL, CLASP2, SMC5, SEPTIN6, RRS1, FBXO28, |
| | NCAPD3, CLASP1, NCAPH, ITGB3BP, BRD4, PES1, GPATCH1, SYCE2, |
| | SUN2, AHCTF1, BOD1L1, NSL1, L3MBTL1, SS18L1, MLH3, SMC1B, |
| | CHAMP1, KANSL1, BOD1L2, RNF212, NSMCE2, NCAPH2, H2AX, |
| | HMGB1, HMGB2, C14orf39, HNRNPU, HSF1, HSPA2, BIRC5, HUS1, IHO1, |
| | SKA2, NUP43, INCENP, CENPS, CENPW, LIG4, MAD2L1, MKI67, MLH1, |
| | STAG3L3, STAG3L2, MSH4, SEPTIN2, NEK2, EID3, ORC2, SYCP3, |
| | KMT5B, DYNC1LI, CHMP1A, SPOUT1, PHF2, PLK1, STAG3L1, ATRX, |
| | NSMCE4A, NDE1, ERCC6L, NCAPG2, SPDL1, PINX1, PPP1CC, ZWILCH, |
| | RIF1, MIS18BP, HJURP, NUP133, CENPN, TEX14, TEX12, TEX11, |
| | NSMCE3, KNL1, NUP107, PELI1, SPC25, CCNB1IP, RAD1, RAD9A, |
| | RAD21, RAD51, RANGAP1, RGS12, BLM, CENPK, NCAPG, SETMAR, |
| | RAD21L1, SYCE3, MEAF6, STAG3L4, CENPH, NOL6, BRCA1, BRCA2, |
| | AURKA, AURKC, SUV39H1, SYCP1, SYN1, BUB1, DYNLT3, BUB1B, |
| | TOP2A, TP53BP1, TTN, TUBG1, MEIKIN, UBE2I, ZNF207, MIS12, CENPM, |
| | NUP37, CENPO, SMC6, CENPU, NUP85, DSN1, CENPT, NDEL1, CDT1, |
| | SEH1L, SMC1A, NUF2, MAD1L1, HORMAD, PHF6, NIFK, DCTN5, KIF2B, |
| | SMARCA5, FKBP6, KMT5C, CCNB1, TOP3B, RSPH1, BAZ1B, KNSTRN, |
| | SMC3, BOD1, ZW10, BUB3, AURKB, ZNF276, SYCE1, MACROH, KNTC1, |
| | RASSF2, CKAP5, SEPTIN7, NCAPD2, REC8 |
| GO_CONDENSED_{—} | SYCP2, NDC80, CFDP1, CEBPB, CENPA, CENPB, CENPC, SPAG5, CENPE, |
| CHROMOSOME_{—} | CENPF, DCTN6, KIF2C, ZWINT, PMF1, DCTN3, CBX3, ANAPC16, |
| CENTROMERIC_{—} | NUDCD2, CSNK1A1, SPC24, SGO2, SGO1, DYNC1I1, CENPV, CENPX, |
| REGION | SKA1, SKA3, SYCP2L, LRWD1, CLASP2, SEPTIN6, FBXO28, NCAPD3, |
| | CLASP1, ITGB3BP, GPATCH11, AHCTF1, BOD1L1, NSL1, SS18L1, |
| | CHAMP1, KANSL1, BOD1L2, HNRNPU, HSF1, BIRC5, SKA2, NUP43, |
| | INCENP, CENPS, CENPW, MAD2L1, SEPTIN2, NEK2, ORC2, KMT5B, |
| | DYNC1LI, SPOUT1, PHF2, PLK1, ATRX, NDE1, ERCC6L, SPDL1, PINX1, |
| | PPP1CC, ZWILCH, MIS18BP, HJURP, NUP133, CENPN, TEX14, KNL1, |
| | NUP107, PELI1, SPC25, RAD21, RANGAP1, CENPK, NCAPG, RAD21L1, |
| | MEAF6, CENPH, AURKA, AURKC, BUB1, DYNLT3, BUB1B, TP53BP1, |
| | MEIKIN, ZNF207, MIS12, CENPM, NUP37, CENPO, CENPU, NUP85, DSN1, |
| | CENPT, NDEL1, CDT1, SEH1L, SMC1A, NUF2, MAD1L1, PHF6, DCTN5, |
| | KIF2B, KMT5C, CCNB1, KNSTRN, BOD1, ZW10, BUB3, AURKB, ZNF276, |
| | KNTC1, RASSF2, CKAP5, SEPTIN7, NCAPD2, REC8 |
| GO_CONDENSED_{—} | NDC80, CENPF, SKA1, SKA3, BOD1L1, BOD1L2, SKA2, PLK1, SPDL1, |
| CHROMOSOME_{—} | NUP133, BUB1, BUB1B, CCNB1, BOD1 |
| OUTER_{—} | |
| KINETOCHORE | |
| GO_CONDENSIN_{—} | SMC4, SMC2, NCAPD3, NCAPH, NCAPH2, NCAPG2, NCAPG, NCAPD2 |
| COMPLEX | |
| GO_ER_NUCLEUS_{—} | BCL2L11, LPCAT3, AGR2, ERLIN1, ERLIN2, ATF6B, DDIT3, EIF2S1, |
| SIGNALING_{—} | ATF6, SCAP, PPP1R15A, AMFR, GSK3B, HSPA5, IGFBP1, CXCL8, INSIG1, |
| PATHWAY | ASNS, ATF3, ATF4, NCK1, NFE2L2, ATP2A2, ZBTB7B, INSIG2, MBTPS2, |
| | TMCO1, ARHGEF10L, TMEM33, FBXW7, SELENOS, CCDC47, PTPN1, |
| | PTPN2, CCL2, BOK, SREBF1, SREBF2, TP53, HSP90B1, WFS1, XBP1, |
| | C12orf49, CALR, ATG10, EIF2A, NCK2, PPP1R15B, MBTPS1, EIF2AK3, |
| | HERPUD1 |
| GO_ESTABLISHMENT_{—} | SPRY1, SPRY2, NDC80, CENPA, MISP, UBXN2B, DCTN1, FGF10, CLASP2, |
| OF_MITOTIC_{—} | FBXW11, CLASP1, GJA1, GPSM2, HTT, ITGB1, KPNB1, INSC, MAD2L1, |
| SPINDLE_{—} | NUMA1, PAFAH1B1, PAX6, NUSAP1, PLK1, NDE1, SPDL1, CDK5RAP2, |
| LOCALIZATION | NSFL1C, BCCIP, DYNLT1, MCPH1, NDEL1, SAPCD2, ARHGEF2, ZW10, |
| | ESPL1 |
| GO_EUKARYOTIC_{—} | EIF2S1, EIF2B1, EIF2B4, EIF2B3, EIF2B2, EIF2B5 |
| TRANSLATION_{—} | |
| INITIATION_{—} | |
| FACTOR_2B_{—} | |
| COMPLEX | |
| GO_KINETOCHORE | NDC80, CFDP1, DCTN2, CENPA, CENPC, SPAG5, CENPE, CENPF, DCTN6, |
| | SUGT1, KIF2C, ZWINT, PMF1, DCTN3, ANAPC1, NUDCD, CSNK1A, |
| | SPC24, SGO2, SGO1, DCTN1, DYNC1I, DYNC1L, CENPV, CENPX, SKA1, |
| | SKA3, LRWD1, CLASP2, SEPTIN, FBXO28, NUP160, FBXW11, CLASP1, |
| | ITGB3B, CBX5, CENPI, GPATCH, 1, AHCTF1, BOD1L1, NSL1, SIN3A, |
| | SS18L1, MTBP, CHAMP1, KANSL1, BOD1L2, HNRNPU, APC, HSF1, |
| | BIRC5, SKA2, NUP43, INCENP, CENPS, KIF22, CENPW, MAD2L1, PPP1R1, |
| | SEPTIN, NEK2, NUP98, ORC2, PAFAH1, TRAPPC, DYNC1L, DCTN4, |
| | SPOUT1, PHF2, PLK1, NDE1, ERCC6L, SPDL1, PINX1, PPP1CC, ZWILCH, |
| | CENPQ, MIS18B, HJURP, NUP133, CENPN, TEX14, PSEN1, PSEN2, KNL1, |
| | NUP107, PELI1, SPC25, RANGAP, CLIP1, SEC13, CENPK, MEAF6, CENPH, |
| | SUMO3, BUB1, DYNLT3, BUB1B, TP53BP, TPR, TTK, MEIKIN, XPO1, |
| | ZNF207, MIS12, CENPM, NUP37, CENPO, CENPU, NUP85, DSN1, CENPT, |
| | NDEL1, CDT1, SEH1L, KIF18A, SMC1A, NUF2, MAD1L1, KAT8, PHF6, |
| | DCTN5, KIF2B, DYNLL1, KAT2B, CCNB1, KNSTRN, BOD1, ZW10, BUB3, |
| | AURKB, ZNF276, KNTC1, RASSF2, CKAP5, SEPTIN, REC8 |
| GO_MCM_{—} | MCMDC2, MMS22L, MCM9, MCM2, MCM3, MCM4, MCM5, MCM6, |
| COMPLEX | MCM7, TONSL, MCMBP, MCM8 |
| GO_MICROTUBULE_{—} | SPAG5, GAS2L1, MAPRE2, KIF2C, KIF18B, DCTN1, MAPRE1, MAPRE3, |
| PLUS_END | CLASP1, GAS2L2, CLIP3, NCKAP5, MYO5A, NUMA1, CDK5RAP2, |
| | SLAIN2, NCKAP5L, CLIP1, DST, CLIP2, CLIP4, KNSTRN, CKAP5 |
| GO_MITOTIC_{—} | UBE2DNL, TOM1L1, SMC4, NME6, CDKN1B, AKAP8, STAG1, KATNB1, |
| NUCLEAR_{—} | NPM2, NDC80, PRMT5, MAD2L2, TACC3, PIBF1, DCTN2, SMC2, CENPC, |
| DIVISION | USP16, SPAG5, CENPE, CENPF, STAG2, PTTG2, NEK6, KIF2C, RCC1, |
| | TENT4A, UBE2C, CHEK1, ZWINT, CHEK2, BIRC8, CDCA5, PHB2, MISP, |
| | CCSAP, CHMP4B, DIS3L2, UBXN2B, TOM1L2, KIF18B, PHF13, SGO2, |
| | SGO1, SPICE1, DAPK3, TOGARAM2, CDC14C, DMRT1, DRD3, DUSP1, |
| | EDN1, EDN3, EGF, KLHDC8B, EPS8, EREG, FGF8, SIRT2, TPX2, PDS5B, |
| | CUL9, TOGARAM1, CLASP2, POGZ, SMC5, FLNA, ABRAXAS2, RRS1, |
| | PDS5A, NCAPD3, CLASP1, OBSL1, MAU2, NCAPH, NUP62, KIF4A, |
| | CDC26, EPGN, NIPBL, ANAPC15, NSL1, SH2B1, CHMP2B, L3MBTL1, |
| | PTTG3P, FBXO5, AKAP8L, MTBP, TUBG2, VPS4A, CHMP2A, UBE2S, |
| | GOLGA2, CHAMP1, KIF4B, NSMCE2, FBXO43, RGCC, CHMP4A, |
| | RACGAP1, NCAPH2, ANAPC4, HNRNPU, APC, HSPA1A, HSPA1B, XIAP, |
| | BIRC5, IGF1, IGF2, GEN1, IK, IL1A, IL1B, INCENP, INS, INSR, KIF2A, |
| | KIF11, KIFC1, KIF25, KIF22, KPNB1, RHOA, LRP5, MAD2L1, MKI67, |
| | MZT1, MYBL2, ATM, DRG1, NEK2, NEUROG1, NFE2L1, NUMA1, |
| | WRAP73, RMDN1, DYNC1LI1, CHMP1A, NUSAP1, ANAPC5, ANAPC7, |
| | LCMT1, CHMP5, ANAPC11, SIRT7, PDGFB, PDGFRB, PIN1, PLK1, ANLN, |
| | FBXW5, ATRX, INO80, MAP10, NDE1, NCAPG2, SPDL1, CHTF8, PINX1, |
| | AURKAIP1, PHIP, CEP192, CDCA8, MSTO1, PPP2R1A, SMPD3, SLF2, |
| | CDK5RAP2, RIOK2, PCID2, PPP2R2D, NSFL1C, TEX14, BCCIP, PSMG2, |
| | PDXP, CHMP1B, HECW2, RAD21, RAN, RANBP1, RB1, CENPK, NCAPG, |
| | RAD21L1, ANAPC1, CEP85, BMP4, BMP7, SPAST, AURKA, AURKC, BTC, |
| | BUB1, BUB1B, TGFA, TPR, TTK, TTN, TUBG1, HIRA, XRCC3, ZNF207, |
| | MIS12, DSCC1, CEP97, CHMP6, BORA, MAP9, DSN1, ANKRD53, NAA50, |
| | AAAS, NDEL1, CDT1, SEH1L, KIF18A, SMC1A, NAA10, NUF2, MAD1L1, |
| | HASPIN, CCDC8, USP44, ABRAXAS1, SLF1, CUL3, PPP1R9B, PSRC1, |
| | KLHL22, CDC14B, CDC14A, CAV2, CDK13, TNKS, BECN1, CDC23, |
| | RAB11A, SPHK1, CDC16, CCNB1, KNSTRN, PRC1, PKMYT1, KIFC2, |
| | SMC3, BOD1, CTDP1, CHMP7, ZW10, BUB3, AURKB, PTTG1, CHMP4C, |
| | TRIP13, KIF3B, CD28, KIF23, VPS4B, MACROH2A1, KIF20B, MAD2L1BP, |
| | ARHGEF10, ESPL1, KNTC1, DLGAP5, CUL7, CDC6, CDC20, NCAPD2, |
| | KIF14, CDC25C, CDC27, CDC42, REC8 |
| GO_MITOTIC_{—} | STAG1, TACC3, TADA3, SPAG5, CENPE, STAG2, TPPP, KATNA1, EFHC1, |
| SPINDLE | CLTC, MISP, KDF1, CCSAP, NUDCD2, KIF18B, CKAP2L, RMDN2, DCTN1, |
| | TOGARA, 2, CDC14C, DIAPH1, ECT2, EML1, FLCN, CTTN, EPB41, SKA1, |
| | SKA3, MAPRE1, MAPRE3, SIRT2, TPX2, MAPKBP, TOGARA, 1, CLASP2, |
| | CLASP1, NUP62, EML2, HEPACA, EML3, ASPM, KAT2A, GEM, EML4, |
| | GOLGA2, RACGAP, GPSM2, HNRNPU, HSF1, NEXMIF, DCDC1, IK, |
| | CXCR2, KIF11, KIFC1, KIF22, LIMK2, MAD2L1, MAK, MAP4, NUMA1, |
| | WRAP73, RMDN1, NIN, NUSAP1, NGRN, DCDC2, SPOUT1, RTRAF, |
| | YPEL5, PKD2, PKHD1, MAP10, ODAM, RMDN3, MAP11, YEATS2, |
| | CDK5RA, RCC2, MAPK1, BCCIP, HECW2, RANGAP, AGBL5, RPS3, |
| | SLC34A1, AURKA, TBL1X, DYNLT3, TFDP2, TPR, TAF1D, HAUS3, |
| | OR2A4, TBL1XR1, KATNBL, MAP9, ATAT1, EFHC2, AAAS, FAM83D, |
| | KIF18A, CAPG, SMC1A, CDC7, MAD1L1, FAM161, CUL3, KIF2B, RAE1, |
| | KLHL22, PKP4, IKBKG, CEP295, CDC14B, CDC., DYNLL1, TNKS, HDAC3, |
| | KNSTRN, PRC1, KIFC2, SMC3, AURKB, TBCK, KIF23, KIF20B, NCOR1, |
| | IQCB1, ESPL1, CDK1, CEP170, CDC42 |
| GO_NUCLEAR_{—} | SYCE1L, UBE2DNL, DDX11L8, RNF212B, SMC4, ACTR3, ACTR2, MEI4, |
| CHROMOSOME_{—} | AKAP8, STAG1, KATNB1, SYCP2, NDC80, MAD2L2, TACC3, PIBF1, |
| SEGREGATION | DCTN2, SMC2, CENPC, P3H4, SPAG5, CENPE, CENPF, CTCF, STAG3, |
| | STAG2, PTTG2, NEK6, KIF2C, TENT4A, UBE2C, ZWINT, DMC1, CDCA5, |
| | PHB2, ESCO1, RMI2, CHMP4B, DIS3L2, M1AP, CORT, TERB2, KIF18B, |
| | EME1, KASH5, PHF13, CTNNB1, HORMAD, MEI1, TTL, SGO2, SGO1, |
| | SPICE1, ESCO2, MCMDC2, SHOC1, HFM1, DDX11, DYNC1H1, DUSP1, |
| | ECT2, AGO4, EME2, KLHDC8B, CENPX, ERCC4, FANCD2, FEN1, |
| | MAPK15, SIRT2, PDS5B, WAPL, POGZ, SMC5, ABRAXAS, RRS1, PDS5A, |
| | NCAPD3, SUN1, MAU2, NCAPH, SPO11, NUP62, KIF4A, CDC26, MEIOB, |
| | ANKRD31, SYCE2, NIPBL, ANAPC15, NSL1, CHMP2B, PTTG3P, FBXO5, |
| | GEM, AKAP8L, MLH3, SMC1B, TUBG2, VPS4A, CHMP2A, MAJIN, |
| | CHAMP1, TERB1, MEIOC, RNF212, KIF4B, NSMCE2, CHMP4A, |
| | RACGAP1, NCAPH2, ANAPC4, C14orf39, HNRNPU, APC, IHO1, GEN1, IK, |
| | INCENP, CENPS, KIFC1, KIF25, KIF22, KPNB1, MAD2L1, MLH1, MOS, |
| | MRE11, DDX12P, STAG3L3, STAG3L2, MSH4, ATM, NEK2, NUMA1, |
| | WRAP73, TRAPPC1, RMDN1, DYNC1LI1, CHMP1A, NUSAP1, ANAPC5, |
| | ANAPC7, LCMT1, CHMP5, ANAPC11, SIRT7, PLK1, STAG3L1, ATRX, |
| | INO80, MAP10, NCAPG2, SPDL1, CHTF8, PINX1, CDCA8, MSTO1, CENPQ, |
| | PPP2R1A, FBXW7, NDC1, SLF2, CDK5RAP, RIOK2, PCID2, HDAC8, RCC2, |
| | TEX15, TEX14, TEX12, TEX11, FMN2, PRDM9, PSMG2, KNL1, CHMP1B, |
| | HECW2, FANCM, CCNB1IP1, RAD21, RAD51C, RAN, RB1, CENPK, |
| | NCAPG, SFPQ, RAD21L1, SYCE3, ANAPC1, STAG3L4, AURKC, SYCP1, |
| | BUB1, BUB1B, TERF1, TOP2A, TOP2B, TPR, TTK, TTN, TUBG1, MEIKIN, |
| | HIRA, UBE2B, XRCC3, ZNF207, MIS12, DSCC1, BAG6, CHMP6, MCMBP, |
| | DSN1, ANKRD53, RMI1, MUS81, NAA50, FAM83D, CDT1, SEH1L, KIF18A, |
| | SMC1A, NAA10, AXIN2, NUF2, MAD1L1, HASPIN, BRIP1, HORMAD, |
| | USP44, ABRAXAS, SLF1, SLX4, CUL3, KIF2B, SPATA22, PSRC1, KLHL22, |
| | MAEL, TNKS, BECN1, CDC23, RAB11A, CDC16, CCNB1, CCNE1, |
| | KNSTRN, PRC1, LATS1, SMC3, BOD1, CCNE2, CHMP7, ZW10, BUB3, |
| | AURKB, PTTG1, CHMP4C, TRIP13, SYCE1, KIF23, VPS4B, MACROH2, |
| | ESPL1, DLGAP5, CDC6, CDC20, NCAPD2, KIF14, CDC27, CDC42, REC8 |
| GO_ORGANELLE_{—} | ACOT8, SYCE1L, UBE2DNL, BCL2L11, TOM1L1, RNF212B, SMC4, |
| FISSION | DNM1L, ACTR3, ACTR2, RAD50, MEI4, NME6, CDKN1B, AKAP8, STAG1, |
| | KATNB1, NPM2, SYCP2, NDC80, PRMT5, MAD2L2, TACC3, PIBF1, |
| | DCTN2, SMC2, CENPC, USP16, P3H4, SPAG5, CENPE, CENPF, STAG3, |
| | STAG2, PTTG2, NEK6, PPARGC1A, KIF2C, TDRKH, RCC1, TENT4A, |
| | UBE2C, PRDM7, CHEK1, CRYZL2P-SEC16B, ZWINT, DMC1, CHEK2, |
| | BIRC8, MTFR2, CDCA5, PHB2, KLHDC3, CKS2, LRRK2, TDRD9, CNTD1, |
| | MIEF2, MISP, CCSAP, CHMP4B, DIS3L2, M1AP, CORT, COX10, ASZ1, |
| | UBXN2B, TAF1L, PSMA8, TERB2, TOM1L2, KIF18B, EME1, KASH5, |
| | PHF13, HORMAD2, MEI1, SGO2, SGO1, SPICE1, MCMDC2, SHOC1, |
| | C11orf65, DAPK3, DAZL, DCN, HFM1, TOGARAM2, CDC14C, DHODH, |
| | DNM1, DMRT1, DNM2, DRD3, DUSP1, EDN1, EDN3, AGO4, EGF, EME2, |
| | KLHDC8B, CENPX, EPS8, EREG, ERCC4, FANCA, FANCD2, SYCP2L, |
| | FGF8, MAPK15, SIRT2, TPX2, PDS5B, WAPL, CUL9, TOGARAM1, |
| | CLASP2, POGZ, SMC5, FLNA, ABRAXAS2, RRS1, PLCB1, PDS5A, |
| | DDHD2, UBR2, NCAPD3, CLASP1, SUN1, OBSL1, MAU2, NCAPH, SPO11, |
| | NUP62, KIF4A, SPDYA, CDC26, MEIOB, EPGN, ANKRD31, SYCE2, |
| | RAD54B, NIPBL, CATSPERZ, ANAPC15, ASPM, NSL1, SH2B1, CHMP2B, |
| | L3MBTL1, DNM3, PTTG3P, FBXO5, AKAP8L, MLH3, MTBP, TUBG2, |
| | VPS4A, CHMP2A, UBE2S, GOLGA2, GPR3, MAJIN, CHAMP1, TERB1, |
| | MEIOC, RNF212, KIF4B, NSMCE2, FBXO43, RGCC, CHMP4A, RACGAP1, |
| | NCAPH2, PSMC3IP, ANAPC4, DNMT3L, SLC2A8, C14orf39, HNRNPU, |
| | APC, HSF1, HSPA1A, HSPA1B, HSPA2, XIAP, BIRC5, NANOS2, IHO1, |
| | TUBB8, IGF1, IGF2, GEN1, IK, IL1A, IL1B, INCENP, ING2, INS, INSR, |
| | CENPS, KDR, KIF2A, KIF11, KIFC1, KIF25, KIF22, KPNB1, RHOA, LIF, |
| | TEX19, LRP5, MAD2L1, MAPT, MKI67, MLH1, MOS, MRE11, MZT1, |
| | MSH3, MSH4, MSH5, MSX1, MSX2, GGNBP1, MX1, MX2, MYBL1, |
| | MYBL2, ATM, DRG1, NEK2, NEUROG1, NFE2L1, NUMA1, WEE2, OPA1, |
| | WRAP73, SYCP3, PRKN, FIS1, RMDN1, DYNC1LI1, CHMP1A, NUSAP1, |
| | FZR1, PDE3A, ANAPC5, ANAPC7, LCMT1, CHMP5, ANAPC11, MTFP1, |
| | SIRT7, PDGFB, CYRIB, PDGFRB, PIN1, PLK1, GDAP1, WNT4, ANLN, |
| | MOV10L1, FBXW5, MIEF1, DDX4, ATRX, INO80, MAP10, MARCHF5, |
| | NDE1, NCAPG2, SPDL1, CHTF8, PINX1, AURKAIP1, PHIP, PIWIL2, |
| | CEP192, CDCA8, MSTO1, PPP2R1A, SMPD3, NDC1, SLF2, PRKAR1A, |
| | VPS35, CDK5RAP2, RIOK2, PCID2, PPP2R2D, NSFL1C, TEX15, TEX14, |
| | TEX12, TEX11, MTFR1L, CYP26B1, BCCIP, FMN2, SPIRE1, MFF, PRDM9, |
| | PSMG2, PDXP, CHMP1B, PSMD13, HECW2, FANCM, CCNB1IP1, RAD1, |
| | PEX19, RAD21, RAD51, RAD51C, RAD51B, RAD51D, RAN, RANBP1, RB1, |
| | DMRTC2, FIGNL1, CENPK, NCAPG, RAD21L1, BTBD18, SYCE3, INF2, |
| | ANAPC1, CEP85, PINK1, TMEM135, BMP4, BMP7, BNIP3, SPAST, BRCA2, |
| | BRDT, STAT2, AURKA, AURKC, SYCP1, BTC, BUB1, BUB1B, TERF1, |
| | TGFA, TOP2A, TOP2B, TPR, TTK, TTN, TUBG1, MEIKIN, HIRA, UBB, |
| | UBE2B, WNT5A, XRCC3, ZNF711, ZNF207, MIS12, DSCC1, BAG6, MUL1, |
| | CEP97, CHMP6, C11orf80, BORA, MAP9, DSN1, ANKRD53, RMI1, MYO19, |
| | MUS81, NAA50, DDHD1, AAAS, CALR, NDEL1, CDT1, SHCBP1L, SEH1L, |
| | KIF18A, SMC1A, NAA10, NUF2, MAD1L1, HASPIN, CCDC8, BRIP1, |
| | MND1, HORMAD1, USP44, ABRAXAS1, SLF1, RAD54L, SLX4, SPIRE2, |
| | CUL3, PPP1R9B, SPATA22, PSRC1, KLHL22, MASTL, MAEL, AP3B1, |
| | CDC14B, CDC14A, CAV2, CDK13, TNKS, BECN1, CDC23, RAB11A, |
| | PEX11B, PEX11A, SPHK1, CDC16, CCNA1, CCNB1, CCNE1, SEC16B, |
| | KNSTRN, PRC1, MCU, PKMYT1, KIFC2, SLC25A46, SMC3, BOD1, CCNE2, |
| | CTDP1, TDRD12, CHMP7, ZW10, BUB3, AURKB, PTTG1, CHMP4C, |
| | PEX11G, TRIP13, SYCE1, KIF3B, CD28, KIF23, VPS4B, MACROH2A, |
| | KIF20B, MAD2L1BP, ARHGEF10, MTFR1, MARF1, ESPL1, KNTC1, |
| | DLGAP5, CUL7, WASHC5, CDC6, CDC20, NCAPD2, KIF14, CDC25B, |
| | CDC25C, CDC27, CDC42, REC8 |
| GO_PROTEIN_{—} | NDC80, CENPA, CTCF, MTBP, CHAMP1, IK, TRAPPC12, SPDL1, CENPQ, |
| LOCALIZATION_{—} | RCC2, KNL1, RB1, TTK, MIS12, CENPT, CDT1, HASPIN, BOD1, ZW10, |
| TO_CHROMOSOME_{—} | BUB3, AURKB, CDK1 |
| CENTROMERIC_{—} | |
| REGION | |
| GO_REGULATION_{—} | SPAG5, ECT2, SIRT2, RACGAP1, HNRNPU, APC, NEK2, RCC2, BECN1, |
| OF_ATTACHMENT_{—} | CCNB1, KNSTRN, AURKB, CDC42 |
| OF_SPINDLE_{—} | |
| MICROTUBULES_{—} | |
| TO_KINETOCHORE | |
| GO_REGULATION_{—} | UBE2DNL, MAGEA10-MAGEA5, NDC80, MAD2L2, TACC3, SPAG5, |
| OF_CHROMOSOME_{—} | CENPE, CENPF, CTCF, PTTG2, NEK6, KIF2C, UBE2C, CDCA5, RMI2, |
| SEGREGATION | CSNK2A1, CSNK2A2, CTNNB1, TTL, DDX11, DYNC1H1, DUSP1, ECT2, |
| | FEN1, MAPK15, SIRT2, WAPL, SMC5, PUM2, CDC26, NIPBL, ANAPC15, |
| | PTTG3P, FBXO5, NSMCE2, RACGAP1, ANAPC4, HNRNPU, APC, IHO1, |
| | GEN1, IK, MAD2L1, MAGEA5, MKI67, MOS, ATM, NEK2, NUMA1, |
| | DYNC1LI1, ANAPC5, ANAPC7, LCMT1, ANAPC11, PLK1, ATRX, SPDL1, |
| | SLF2, CDK5RAP2, RIOK2, PCID2, HDAC8, RCC2, TEX14, RAD18, PSMG2, |
| | HECW2, RAD21, RB1, SFPQ, ANAPC1, BUB1, BUB1B, TPR, TTK, XRCC3, |
| | ZNF207, SMC6, CDT1, NAA10, AXIN2, MAD1L1, HORMAD1, USP44, |
| | SLF1, CUL3, KIF2B, KLHL22, TNKS, BECN1, CDC23, CDC16, CCNB1, |
| | KNSTRN, ZW10, BUB3, AURKB, PTTG1, TRIP13, MACROH2A1, PUM1, |
| | ESPL1, DLGAP5, CDC6, CDC20, CDC27, CDC42 |
| GO_REGULATION_{—} | PARP3, TACC3, SPAG5, RCC1, CLTC, CCSAP, CHMP4B, DCTN1, |
| OF_SPINDLE_{—} | DYNC1H1, MAPK15, TPX2, NUP62, CHMP2B, SENP6, CHMP2A, GNAI1, |
| ORGANIZATION | GPSM2, HNRNPU, HSPA1A, HSPA1B, DRG1, NUMA1, CHMP1A, CHMP5, |
| | PKD1, PLK1, CHMP1B, RNF4, TPR, FSD1, CEP97, BORA, MAP9, |
| | ANKRD53, PSRC1, RAE1, CHMP4C, VPS4B |
| GO_SISTER_{—} | UBE2DNL, DDX11L8, SMC4, AKAP8, STAG1, KATNB1, NDC80, MAD2L2, |
| CHROMATID_{—} | TACC3, PIBF1, DCTN2, SMC2, CENPC, SPAG5, CENPE, CENPF, CTCF, |
| SEGREGATION | STAG3, STAG2, PTTG2, NEK6, KIF2C, TENT4A, UBE2C, ZWINT, CDCA5, |
| | PHB2, ESCO1, RMI2, CHMP4B, DIS3L2, KIF18B, PHF13, CTNNB1, |
| | HORMAD2, SGO2, SGO1, ESCO2, DDX11, DUSP1, FEN1, MAPK15, PDS5B, |
| | WAPL, POGZ, SMC5, RRS1, PDS5A, NCAPD3, MAU2, NCAPH, NUP62, |
| | KIF4A, CDC26, NIPBL, ANAPC15, NSL1, CHMP2B, PTTG3P, FBXO5, |
| | AKAP8L, SMC1B, TUBG2, VPS4A, CHMP2A, CHAMP1, KIF4B, NSMCE2, |
| | CHMP4A, RACGAP1, NCAPH2, ANAPC4, HNRNPU, APC, GEN1, IK, |
| | INCENP, KIFC1, KIF25, KIF22, KPNB1, MAD2L1, MOS, MRE11, DDX12P, |
| | STAG3L3, STAG3L2, ATM, NEK2, NUMA1, WRAP73, RMDN1, DYNC1LI1, |
| | CHMP1A, NUSAP1, ANAPC5, ANAPC7, LCMT1, CHMP5, ANAPC11, PLK1, |
| | STAG3L1, ATRX, INO80, MAP10, NCAPG2, SPDL1, CHTF8, PINX1, |
| | CDCA8, MSTO1, PPP2R1A, FBXW7, SLF2, CDK5RAP2, RIOK2, PCID2, |
| | HDAC8, TEX14, PSMG2, CHMP1B, HECW2, RAD21, RAD51C, RAN, RB1, |
| | CENPK, NCAPG, SFPQ, RAD21L1, ANAPC1, STAG3L4, AURKC, BUB1, |
| | BUB1B, TOP2A, TOP2B, TPR, TTK, TTN, TUBG1, MEIKIN, HIRA, XRCC3, |
| | ZNF207, MIS12, DSCC1, CHMP6, MCMBP, DSN1, ANKRD53, NAA50, |
| | CDT1, SEH1L, KIF18A, SMC1A, NAA10, AXIN2, NUF2, MAD1L1, HASPIN, |
| | HORMAD1, USP44, SLF1, CUL3, PSRC1, KLHL22, TNKS, BECN1, CDC23, |
| | RAB11A, CDC16, CCNB1, KNSTRN, PRC1, LATS1, SMC3, BOD1, CHMP7, |
| | ZW10, BUB3, AURKB, PTTG1, CHMP4C, TRIP13, KIF23, VPS4B, |
| | MACROH2A1, ESPL1, DLGAP5, CDC6, CDC20, NCAPD2, KIF14, CDC27, |
| | REC8 |
| GO_SPINDLE | NUBP2, KIF20A, FRY, TOPORS, STAG1, MAEA, KATNB1, TUBGCP3, |
| | MAD2L2, TACC3, TADA3, SPAG5, CENPE, CENPF, RGS14, CETN1, PLK4, |
| | STAG2, PLK2, NEK6, TUBGCP2, JTB, SPIN1, CBX1, MAPRE2, KIF2C, |
| | TOPBP1, TPPP, DIDO1, KATNA1, KIF3A, RASSF1, BIRC8, DCTN3, STX1B, |
| | CBX3, EFHC1, TUBGCP5, HAUS1, CLTA, CLTC, NEDD1, DNAAF1, |
| | SEPTIN12, PLK3, MISP, PLK5, KDF1, CCSAP, ARL8A, NUDCD2, UBXN2B, |
| | CRMP1, NEK7, PARP4, MAPK14, CSNK1D, CEP128, KIF18B, KASH5, |
| | CTNNB1, CKAP2L, RMDN2, SGO1, SPICE1, TTC23L, CYLD, DCTN1, |
| | TOGARAM2, DDX11, CDC14C, DIAPH1, DYNC1I1, E4F1, ECT2, |
| | TMEM201, EML1, EMD, CENPV, FLCN, CTTN, EPB41, ERCC2, AKT1, |
| | SKA1, SKA3, MAPK15, CEP162, MAPRE1, KIFAP3, MAPRE3, SIRT2, |
| | TPX2, MAPKBP1, TOGARAM1, CLASP2, SEPTIN6, TTLL12, ABRAXAS2, |
| | MTCL1, TTC28, CLASP1, HAUS5, SPECC1L, ARL2BP, NUP62, KIF4A, |
| | EML2, NEIL2, HEPACAM2, TCTEX1D2, EML3, POC1A, ASPM, BOD1L1, |
| | CCDC69, SPAG8, FBXO5, KAT2A, LATS2, CKAP2, GEM, INVS, TUBG2, |
| | VPS4A, TUBGCP4, PRPF19, EML4, GOLGA2, POC1B, CHAMP1, BOD1L2, |
| | WDR62, NR3C1, RACGAP1, SLC25A5, GPSM2, SAC3D1, ANXA11, |
| | HNRNPU, HSF1, HSPA2, XIAP, HSPB1, BIRC5, NEXMIF, DCDC1, TUBB8, |
| | SKA2, APP, IK, CXCR2, INCENP, KIF2A, KIF11, KIFC1, KIF22, LIMK2, |
| | KPNA7, ARL3, IRAG2, MAD2L1, MAK, MAP4, MID1, MZT1, MYH9, |
| | MYH10, ATM, SEPTIN2, NEDD9, NEK2, NPM1, NUMA1, ODF2, WRAP73, |
| | PAFAH1B1, RMDN1, SBDS, DYNC1LI1, DCTN4, NIN, NUSAP1, NGRN, |
| | DCDC2, SPOUT1, RTRAF, YPEL5, CIAO2B, PIN4, PKD2, PKHD1, PLK1, |
| | POLB, INO80, MAP10, HAUS6, NDE1, NSUN2, SPDL1, HAUS4, ODAM, |
| | PINX1, PPP2R3C, WDR60, HAUS2, CDCA8, PPP2CA, PPP2CB, TBCCD1, |
| | RMDN3, RIF1, PLEKHG6, MAP1S, ARL8B, MAP11, HAUS7, YEATS2, |
| | MAP7D1, CDK5RAP2, ACOT13, RCC2, MAPK1, NSFL1C, MAP2K5, BEX4, |
| | BCCIP, FMN2, KIF15, BIRC6, MTUS1, HECW2, KLHL42, MICAL3, RAD21, |
| | RANGAP1, RB1, AGBL5, RPS3, MMS19, FAM110C, RASSF10, CEP85, |
| | MZT2A, SLC34A1, SNCG, SPAST, AURKA, AURKC, TBL1X, DYNLT3, |
| | DYNLT1, BUB1B, TERF1, TFDP2, TPR, TPT1, TTK, TUBG1, UMOD, VRK1, |
| | ZNF207, PTP4A1, EVI5, ALMS1, AUNIP, TAF1D, BRCC3, HAUS3, OR2A4, |
| | MAP7D3, TBL1XR1, KATNBL1, SHCBP1, CSPP1, BORA, MAP9, NUP85, |
| | ATAT1, DSN1, ANKRD53, MZT2B, CALM1, CEP63, EFHC2, CALM2, |
| | CALM3, CEP44, AAAS, NDEL1, FAM83D, SHCBP1L, KIF18A, CLTCL1, |
| | CAPG, SMC1A, CAPN6, CDC7, KATNAL2, FAM110A, MAD1L1, HASPIN, |
| | KATNAL1, UXT, FAM161A, RAB11FIP4, CUL3, KBTBD8, KIF2B, PSRC1, |
| | RAE1, KLHL22, CEP89, WDR73, CEP19, PKP4, IKBKG, FBF1, TUBGCP6, |
| | CEP295, CDC14B, CDC14A, DYNLL1, TNKS, RAB11A, HDAC3, CDC16, |
| | CCNB1, RSPH1, KNSTRN, PRC1, CEP95, UNC119, KIFC2, LATS1, SMC3, |
| | BOD1, CTDP1, ARHGEF2, ZW10, AURKB, HAUS8, TBCK, KIF3B, CIAO1, |
| | KIF23, VPS4B, KIF20B, MAD2L1BP, NCOR1, IQCB1, ESPL1, CEP104, |
| | KNTC1, DLGAP5, CKAP5, CDK1, CEP350, CEP170, SEPTIN7, CDC6, |
| | KLHL21, CDC20, KIF14, CDC25B, CDC27, CDC42 |
| GO_SPINDLE_{—} | STAG1, TUBGCP3, PIBF1, SPAG5, STAG2, NEK6, TUBGCP2, MAPRE2, |
| ASSEMBLY | RCC1, CHEK2, BIRC8, TUBGCP5, HAUS1, MISP, CCSAP, CHMP4B, NEK7, |
| | CSNK1D, KASH5, SPICE1, TOGARAM2, DYNC1H1, TUBB, MAPK15, |
| | MAPRE1, MAPRE3, TPX2, GOLGA8A, TOGARAM1, CLASP2, FLNA, |
| | ABRAXAS2, CLASP1, HAUS5, KIF4A, ASPM, CHMP2B, SENP6, CCDC69, |
| | FBXO5, TUBGCP4, CHMP2A, GOLGA2, KIF4B, RACGAP1, GPSM2, |
| | SAC3D1, HNRNPU, HSPA1A, HSPA1B, XIAP, BIRC5, TUBB8, INCENP, |
| | KIF2A, KIF11, KIFC1, KPNB1, RHOA, CENPW, MLH1, MZT1, GOLGA8B, |
| | MYBL2, DRG1, NEK2, NUMA1, WRAP73, CHMP1A, CHMP5, PLK1, |
| | INO80, MAP10, HAUS6, HAUS4, CEP192, HAUS2, MSTO1, HAUS7, |
| | GOLGA2P5, BCCIP, CHMP1B, STARD9, RNF4, RPS3, AURKA, AURKC, |
| | TPR, ZNF207, HAUS3, CEP97, MAP9, CEP63, AAAS, TUBB1, SMC1A, |
| | ABRAXAS1, LZTS2, TUBGCP6, TNKS, RAB11A, HDAC3, PRC1, KIFC2, |
| | SMC3, AURKB, CHMP4C, HAUS8, KIF3B, KIF23, VPS4B, NCOR1, |
| | ARHGEF10, WASHC5, CDC20 |
| GO_SPINDLE_{—} | PARP3, STAG1, NDC80, TUBGCP3, TACC3, PIBF1, DCTN2, TACC2, |
| ORGANIZATION | SPAG5, CENPE, RGS14, DCTN6, STAG2, PLK2, NEK6, TUBGCP2, |
| | MAPRE2, RCC1, CHD3, TPPP, CHEK2, BIRC8, EFHC1, TUBGCP5, HAUS1, |
| | CLTC, MISP, CCSAP, CHMP4B, NEK7, CSNK1D, KASH5, SPICE1, CEP120, |
| | DCTN1, TOGARAM2, DLG1, DYNC1H1, EML1, TUBB, MAPK15, MAPRE1, |
| | MAPRE3, TPX2, GOLGA8A, TOGARAM1, CLASP2, FLNA, ABRAXAS2, |
| | CLASP1, HAUS5, NUP62, KIF4A, SUN2, POC1A, ASPM, CHMP2B, SENP6, |
| | CCDC69, FBXO5, TUBG2, TUBGCP4, CHMP2A, GNAI1, GOLGA2, WDR62, |
| | KIF4B, NTMT1, RACGAP1, GPSM2, SAC3D1, HNRNPU, HSPA1A, |
| | HSPA1B, XIAP, BIRC5, TUBB8, INCENP, KIF2A, KIF11, KIFC1, KPNB1, |
| | RHOA, CENPW, STMN1, LIMK2, MAP4, MECP2, MLH1, MOS, MZT1, |
| | GOLGA8B, MYBL2, MYH9, DRG1, NEK2, NUMA1, WRAP73, RMDN1, |
| | SBDS, PCNT, CHMP1A, CHMP5, PKD1, PLK1, ATRX, INO80, MAP10, |
| | HAUS6, HAUS4, CEP192, HAUS2, MSTO1, PPP2R1A, PTPA, TRIM36, |
| | HAUS7, GOLGA2P5, CEP72, INTS13, CENPJ, KIZ, BCCIP, CHMP1B, |
| | SPC25, STARD9, CEP126, RAN, RNF4, RPS3, STIL, CENPH, SPAST, |
| | AURKA, AURKC, TACC1, TBCE, TPR, TTK, TUBG1, UVRAG, VCP, EZR, |
| | ZNF207, AUNIP, FSD1, HAUS3, CEP97, BORA, MAP9, ANKRD53, CEP63, |
| | EFHC2, AAAS, TUBB1, SMC1A, NUF2, ABRAXAS1, LZTS2, PSRC1, RAE1, |
| | TUBGCP6, TNKS, RAB11A, HDAC3, CCNB1, KNSTRN, PRC1, KIFC2, |
| | SMC3, AURKB, CHMP4C, HAUS8, KIF3B, KIF23, VPS4B, NCOR1, |
| | ARHGEF10, ESPL1, DLGAP5, CKAP5, WASHC5, CDC20 |
| GO_SPINDLE_{—} | NUBP2, FRY, TOPORS, STAG1, KATNB1, TUBGCP3, TACC3, SPAG5, |
| POLE | CENPF, RGS14, CETN1, PLK4, STAG2, PLK2, NEK6, TUBGCP2, TOPBP1, |
| | KATNA1, RASSF1, EFHC1, TUBGCP5, HAUS1, NEDD1, DNAAF1, PLK3, |
| | MISP, PLK5, NUDCD2, UBXN2B, NEK7, MAPK14, CEP128, KASH5, |
| | CTNNB1, CKAP2L, RMDN2, SGO1, DCTN1, DDX11, CDC14C, DYNC1I1, |
| | TMEM201, EML1, EMD, TPX2, MAPKBP1, ABRAXAS2, MTCL1, TTC28, |
| | NUP62, TCTEX1D2, POC1A, ASPM, BOD1L1, LATS2, CKAP2, VPS4A, |
| | TUBGCP4, GOLGA2, POC1B, BOD1L2, WDR62, GPSM2, HNRNPU, HSF1, |
| | IK, KIF2A, KIF11, IRAG2, MAD2L1, NEDD9, NEK2, NPM1, NUMA1, ODF2, |
| | RMDN1, SBDS, DYNC1LI1, DCTN4, NIN, SPOUT1, YPEL5, PLK1, MAP10, |
| | NDE1, SPDL1, WDR60, PPP2CA, PPP2CB, TBCCD1, RMDN3, PLEKHG6, |
| | CDK5RAP2, NSFL1C, BEX4, BCCIP, BIRC6, RAD21, FAM110C, RASSF10, |
| | CEP85, AURKA, AURKC, TPT1, TUBG1, UMOD, ALMS1, AUNIP, BRCC3, |
| | OR2A4, KATNBL1, CSPP1, DSN1, ANKRD53, CALM1, CEP63, CALM2, |
| | CALM3, CEP44, AAAS, FAM83D, SMC1A, KATNAL2, FAM110A, |
| | MAD1L1, KATNAL1, UXT, FAM161A, CUL3, PSRC1, RAE1, KLHL22, |
| | CEP89, WDR73, CEP19, PKP4, IKBKG, FBF1, TUBGCP6, CDC14B, |
| | CDC14A, TNKS, RAB11A, CCNB1, KNSTRN, PRC1, CEP95, UNC119, |
| | LATS1, SMC3, BOD1, CTDP1, ZW10, AURKB, HAUS8, VPS4B, KIF20B, |
| | CEP104, KNTC1, DLGAP5, CKAP5, CDC6, KLHL21, CDC20, CDC25B |
| GO_VITAMIN_{—} | AKR1A1, SLC19A2, MTHFS, MTHFD2, ALDH1L1, SLC46A1, SLC52A3, |
| METABOLIC_{—} | CTRC, GSTO2, CYP2R1, CRABP1, CTRB1, CYB5A, CYP1A1, VKORC1L1, |
| PROCESS | RDH10, CYP3A4, CYP11A1, CYP24A1, CYP26A1, CYP27A1, CYP27B1, |
| | ALDH1L2, MMAA, CLYBL, DHFR, CYB5R3, DHFR2, FGFR1, FOLR1, |
| | FOLR2, FPGS, MTHFD1L, MMACHC, GC, GFI1, CBLIF, TPK1, MMADHC, |
| | GCLC, UBIAD1, PSAT1, HLCS, AOX1, MMAB, CYP26C1, IFNG, IL1B, |
| | LRP2, CYP4F3, ABCC1, CTRB2, MTHFD2L, MTHFD1, MTHFR, MTR, |
| | MTRR, MMUT, NFKB1, PC, PCCA, PCCB, PDZD11, CD320, PIAS4, ENPP1, |
| | PLTP, BCO1, ACP5, PPARD, CYP2W1, ACP3, SLC52A1, PNPO, RFK, |
| | VNN3, LMBRD1, LGMN, PRSS1, PRSS3, CYP26B1, MCCC1, PDXP, |
| | DHFRP1, CYP4F11, ABCD4, RBP1, RBP2, RLBP1, SLC25A19, AASDHPPT, |
| | RPE65, MCCC2, SHMT1, SHMT2, SLC2A1, SLC2A3, SLC19A1, SNAI2, |
| | CYP4F12, SNAI1, BTD, TCN1, TCN2, TNF, TTPA, VDR, VKORC1, THTPA, |
| | SLC52A2, PANK2, CUBN, FLAD1, SLC19A3, FGF23, SLC25A32, AMN, |
| | TKTL1, CYP4F2, PDXK, AKR1C3, CBR1, ALDH1A2, VNN2, VNN1, |
| | SLC5A6, RGN, LRAT, GSTO1, SLC23A2, SLC23A1 |
| GO_WATER_{—} | AKR1A1, SLC19A2, MTHFS, MTHFD2, ALDH1L1, SLC46A1, SLC52A3, |
| SOLUBLE_{—} | CTRC, GSTO2, CTRB1, CYB5A, ALDH1L2, MMAA, CLYBL, DHFR, |
| VITAMIN_{—} | CYB5R3, DHFR2, FOLR1, FOLR2, FPGS, MTHFD1L, MMACHC, CBLIF, |
| METABOLIC_{—} | TPK1, MMADHC, GCLC, PSAT1, HLCS, AOX1, MMAB, ABCC1, CTRB2, |
| PROCESS | MTHFD2L, MTHFD1, MTHFR, MTR, MTRR, MMUT, PC, PCCA, PCCB, |
| | PDZD11, CD320, ENPP1, ACP5, ACP3, SLC52A1, PNPO, RFK, VNN3, |
| | LMBRD1, PRSS1, PRSS3, MCCC1, PDXP, DHFRP1, ABCD4, SLC25A19, |
| | AASDHPPT, MCCC2, SHMT1, SHMT2, SLC2A1, SLC2A3, SLC19A1, BTD, |
| | TCN1, TCN2, THTPA, SLC52A2, PANK2, CUBN, FLAD1, SLC19A3, |
| | SLC25A32, AMN, TKTL1, PDXK, VNN2, VNN1, SLC5A6, RGN, GSTO1, |
| | SLC23A2, SLC23A1 |
| HALLMARK_G2M_{—} | ABL1, AMD1, ARID4A, ATF5, ATRX, AURKA, AURKB, BARD1, BCL3, |
| CHECKPOINT | BIRC5, BRCA2, BUB1, BUB3, KNL1, CASP8AP2, CBX1, CCNA2, CCNB2, |
| | CCND1, CCNF, CCNT1, CDC20, CDC25A, CDC25B, CDC27, CDC45, CDC6, |
| | CDC7, CDK1, CDK4, CDKN1B, CDKN2C, CDKN3, CENPA, CENPE, |
| | CENPF, CHAF1A, CHEK1, CHMP1A, CKS1B, CKS2, CTCF, CUL1, CUL3, |
| | CUL4A, CUL5, DBF4, DDX39A, DKC1, DMD, DR1, DTYMK, E2F1, E2F2, |
| | E2F3, E2F4, EFNA5, EGF, ESPL1, EWSR1, EXO1, EZH2, FANCC, FBXO5, |
| | FOXN3, G3BP1, GINS2, GSPT1, H2AZ2, H2AX, H2AZ1, HIF1A, HIRA, |
| | H2BC12, HMGA1, HMGB3, HMGN2, HMMR, JPT1, HNRNPD, HNRNPU, |
| | HOXC10, HSPA8, HUS1, ILF3, INCENP, KATNA1, KIF11, KIF15, KIF20B, |
| | KIF22, KIF23, KIF2C, KIF4A, KIF5B, KPNA2, KPNB1, LBR, LIG3, LMNB1, |
| | MAD2L1, MAPK14, MARCKS, MCM2, MCM3, MCM5, MCM6, MEIS1, |
| | MEIS2, MKI67, MNAT1, MT2A, MTF2, MYBL2, MYC, NASP, NCL, NDC80, |
| | NEK2, NOLC1, NOTCH2, NUMA1, NUP50, NUP98, NUSAP1, ODC1, ODF2, |
| | ORC5, ORC6, PAFAH1B1, TENT4A, PBK, PDS5B, PLK1, PLK4, PML, |
| | POLA2, POLE, POLQ, PRC1, PRIM2, PRMT5, PRPF4B, PTTG1, PTTG3P, |
| | PURA, RACGAP1, RAD21, RAD23B, RAD54L, RASAL2, RBL1, RBM14, |
| | RPA2, RPS6KA5, SAP30, KMT5A, SFPQ, SLC12A2, SLC38A1, SLC7A1, |
| | SLC7A5, SMAD3, SMARCC1, SMC1A, SMC2, SMC4, SNRPD1, SQLE, |
| | SRSF1, SRSF10, SRSF2, SS18, STAG1, STIL, STMN1, SUV39H1, SYNCRIP, |
| | TACC3, TFDP1, TGFB1, TLE3, TMPO, TNPO2, TOP1, TOP2A, TPX2, |
| | TRA2B, TRAIP, TROAP, TTK, UBE2C, UBE2S, UCK2, UPF1, NSD2, WRN, |
| | XPO1, YTHDC1, MAP3K20 |
| PID_PLK1_{—} | SSPOP, KIF2A, NDC80, AURKA, PPP1R12, PRC1, BUB1, BUB1B, CDC14B, |
| PATHWAY | ROCK2, KIF20A, CDK1, TPT1, CCNB1, TUBG1, PPP2R1A, WEE1, CDC25B, |
| | CDC25C, PLK1, RHOA, PPP1CB, RAB1A, PPP2CA, CENPE, GOLGA2, |
| | CDC20, PAK1, KIZ, ERCC6L, ODF2, SGO1, BORA, CENPU, STAG2, SPC24, |
| | GORASP1, ECT2, CLSPN, INCENP, FBXW11, FBXO5, TPX2, FZR1, NUDC, |
| | NINL |
| PID_SMAD2_{—} | CTDSP2, CTDSPL, MAPK3, MAPK1, NUP214, PPM1A, NUP153, KPNA2, |
| 3PATHWAY | UBE2I, SMAD3, MAP3K1, SMAD4, KPNB1, SMAD2, PIAS4, CTDSP1 |
| REACTOME_{—} | PRKAR2B, MARK4, PAFAH1B, MKS1, OFD1, CC2D2A, SDCCAG8, |
| ANCHORING_OF_{—} | PRKACA, NDE1, CLASP1, DYNC1I2, PCM1, HSP90AA, DYNLL1, HAUS4, |
| THE_BASAL_BODY_{—} | NINL, MAPRE1, CEP76, CEP192, RPGRIP1L, CCP110, RAB11A, CEP152, |
| TO_THE_PLASMA_{—} | TUBB4A, PPP2R1A, CEP41, B9D1, YWHAE, CEP164, CEP72, CEP70, |
| MEMBRANE | ALMS1, NEK2, CNTRL, TCTN3, CEP89, B9D2, CEP250, RAB3IP, TUBA4A, |
| | AKAP9, TTBK2, HAUS8, TUBG1, NPHP4, CEP162, AHI1, ODF2, CDK5RAP, |
| | DCTN3, HAUS2, ACTR1A, NEDD1, CSNK1D, CEP131, PLK4, NPHP1, |
| | CETN2, HAUS6, CEP78, SCLT1, CENPJ, HAUS1, PCNT, TMEM67, CEP57, |
| | PLK1, RAB8A, TUBA1A, C2CD3, SEPTIN2, TCTN2, YWHAG, CDK1, |
| | IQCB1, CEP83, CEP135, DCTN2, CKAP5, SSNA1, CEP97, CEP63, TUBB, |
| | KIF24, TMEM216, TUBB4B, FBF1, TUBB, DYNC1H1, SFI1, CEP290, |
| | DCTN1, TCTN1, CEP43, HAUS7, CSNK1E, HAUS3, HAUS5, YWHAE, |
| | NDE1, SDCCAG8, PRKAR2B, PRKACA |
| REACTOME_{—} | ACTR1A, AKAP9, ALMS1, CCNB1, CCP110, CDK1, CDK11A, CDK11B, |
| CENTROSOME_{—} | CDK5RAP, CENPJ, CEP131, CEP135, CEP152, CEP164, CEP192, CEP250, |
| MATURATION | CEP290, CEP41, CEP57, CEP63, CEP70, CEP72, CEP76, CEP78, CETN2, |
| | CKAP5, CLASP1, CNTRL, CSNK1D, CSNK1E, DCTN1, DCTN2, DCTN3, |
| | DYNC1H1, DYNC1I2, DYNLL1, FGFR1OP, HAUS2, HSP90AA, MAPRE1, |
| | NDE1, NEDD1, NEK2, NINL, NUMA1, ODF2, OFD1, PAFAH1B, PCM1, |
| | PCNT, PLK1, PLK4, PPP2R1A, PRKACA, PRKAR2B, SDCCAG, SFI1, |
| | SSNA1, TUBA1A, TUBA4A, TUBB, TUBB4A, TUBB4B, TUBG1, TUBG2, |
| | TUBGCP2, TUBGCP3, TUBGCP4, TUBGCP5, TUBGCP6, YWHAE, YWHAG |
| REACTOME_{—} | MAD1L1, CDC27, PRKAR2, PAFAH1, PSMB1, NCAPD2, PSMC4, SPAST, |
| M_PHASE | NCAPH2, VRK2, NUP160, CENPQ, TUBG2, SPDL1, PSMA4, OFD1, TPR, |
| | ANAPC4, SDCCAG, NDC1, WAPL, PPP2R5, KIF2A, PSME4, SIRT2, |
| | NUP133, CSNK2A, PRKACA, UBE2D1, SMC1A, NDE1, CLASP1, NUP37, |
| | TUBA3D, DYNC1I, PCM1, HSP90A, NDC80, XPO1, TNPO1, PDS5B, |
| | CHMP2B, SEH1L, ZW10, PSMC5, DYNLL1, ANAPC5, BIRC5, NUDC, |
| | PSME1, HAUS4, NUP50, CDC23, AAAS, PSMD5, NUP188, PSMD8, MAPK1, |
| | CENPM, RANGAP, PSMC6, PSMA3, VRK1, PSMC1, PSMB5, PSMA6, |
| | PSME2, NINL, RAE1, TUBB1, PSMA7, MAPRE1, CHMP4B, LPIN2, CEP76, |
| | CEP192, PSMD10, STAG2, EMD, CENPI, MAPK3, NUP93, CENPT, PSMD7, |
| | UBE2I, CCP110, CEP152, RAB2A, PPP2CB, TUBB4A, PPP2R1, H2AZ2, |
| | CEP41, PSMA2, SMC3, UBE2S, PSMD3, KPNB1, NUP88, PSMD11, |
| | YWHAE, NCAPG, ANAPC1, CEP164, NUP98, PSMD9, NUP107, FBXO5, |
| | CEP72, KIF20A, LMNB1, NUP155, PPP2CA, SMC4, CEP70, GORASP, |
| | CENPA, PSMD14, CHMP3, SUMO1, ALMS1, CDC20, BLZF1, NEK2, NSL1, |
| | CENPF, STAG1, SET, CNTRL, NEK6, NEK9, NUP43, CENPL, MASTL, |
| | TMPO, NCAPH, KIF18A, PDS5A, ZWINT, CENPK, TUBA1B, B9D2, |
| | H2BC11, NUP153, NUP85, PSMF1, CEP250, PSMB2, TUBGCP, NUP214, |
| | TUBA4A, AKAP9, SEM1, ARPP19, PSMA1, SGO1, MAU2, CDC16, |
| | CHMP2A, CLIP1, HAUS8, TUBG1, PSME3, NUP210, RAN, H3-3B, VPS4A, |
| | LPIN3, CCNB1, LPIN1, CDCA8, ESPL1, DYNC1L, NUP42, ODF2, SMC2, |
| | CDK5RA, PSMB7, DCTN3, TUBB2A, TUBB2B, NUMA1, KIF23, KNL1, |
| | HAUS2, RAB1A, CENPO, ACTR1A, NUP54, USO1, CENPE, NEDD1, |
| | NUP58, RB1, KIF2B, CSNK1D, CEP131, PSMB6, PLK4, ITGB3B, KIF2C, |
| | PSMA5, NME7, NUF2, ENSA, LBR, EML4, RPS27A, CENPC, H2BC1, |
| | CDCA5, NCAPG2, HDAC8, MCPH1, CETN2, CHMP7, HAUS6, CEP78, |
| | INCENP, DSN1, UBC, NEK7, NCAPD3, CENPU, CENPJ, MZT2B, TUBA3E, |
| | HAUS1, SPC25, CENPH, RANBP2, AHCTF1, PPP2R5E, CC2D1B, PRKCA, |
| | BUB3, PSMA8, SKA1, NUP205, BUB1B, SEC13, CCNB2, H2BC5, H4C8, |
| | PSMD4, PSMB4, PCNT, TAOK1, PMF1, LMNA, PSMC2, SPC24, LEMD2, |
| | NUP35, H3-3A, SGO2, CLASP2, PSMD6, H2AZ1, MAD2L1, NIPBL, PTTG1, |
| | CHMP4C, RAD21, PSMC3, CEP57, CENPN, PRKCB, NDEL1, PLK1, |
| | GOLGA2, TUBA1A, TUBA1C, MIS12, H3-4, BUB1, YWHAG, UBE2E1, |
| | CDK1, UBB, PHF8, TUBB8B, MZT2A, PSMD1, LEMD3, ZWILCH, CEP135, |
| | RAB1B, UBE2C, PSMD2, DCTN2, CKAP5, CENPS, BANF1, PPP2R2, |
| | CTDNEP, TUBB6, SSNA1, CHMP6, ANAPC2, CDC26, ANKLE2, RPS27, |
| | TUBAL3, AURKB, RCC2, RCC1, H2AC6, H2BC4, IST1, SKA2, CEP63, |
| | TUBB, H3C13, TUBA8, KMT5A, H2AC20, KNTC1, H2BC21, H2BC13, |
| | PSMD13, PPP1CC, ERCC6L, TUBB4B, CENPP, H2AX, POM121, ANAPC7, |
| | H2AC7, H2BU1, H4C3, DYNC1H, H3C12, PSMD12, H4C11, H3C4, H4-16, |
| | H2BC12, TUBA3C, SFI1, CEP290, H3C14, H2AC18, H3C15, PSMB8, |
| | CSNK2B, DCTN1, MZT1, PSMB10, CNEP1R, NUP62, CEP43, HAUS7, |
| | CSNK1E, HAUS3, UBA52, PSMB11, H2BC15, H2BS1, PSMB9, TUBA4B, |
| | H2AJ, HAUS5, CHMP4A, TUBB3, TUBB8, DYNLL2, H4C15, H4C14, |
| | H2AC19, H4C12, H2BC14, H2BC8, H3C8, H2AB1, H2BC6, H4C6, H2BC17, |
| | H3C6, H4C13, H3C11, H2BC9, H3C1, PSMB3, H4C9, H2AC14, H2BC3, |
| | H4C5, H2AC8, H4C4, H2BC7, H3C7, H2AC4, H2BC10, H4C1, H4C2, H3C10, |
| | H3C2, H3C3 |
| REACTOME_{—} | MAD1L1, CDC27, PAFAH1B1, PSMB1, PSMC4, SPAST, VRK2, NUP160, |
| MITOTIC_{—} | CENPQ, SPDL1, PSMA4, ANAPC4, NDC1, WAPL, PPP2R5A, KIF2A, |
| METAPHASE_{—} | PSME4, SIRT2, PPP2R5B, NUP133, UBE2D1, SMC1A, NDE1, CLASP1, |
| AND_ANAPHASE | NUP37, TUBA3D, DYNC1I2, PPP2R5C, NDC80, XPO1, TNPO1, PDS5B, |
| | CHMP2B, SEH1L, ZW10, PSMC5, DYNLL1, ANAPC5, BIRC5, NUDC, |
| | PSME1, CDC23, PSMD5, NUP188, PSMD8, CENPM, RANGAP, PSMC6, |
| | PSMA3, VRK1, PSMC1, PSMB5, PSMA6, PSME2, TUBB1, PSMA7, |
| | MAPRE1, CHMP4B, PSMD10, STAG2, EMD, CENPI, NUP93, CENPT, |
| | PSMD7, UBE2I, PPP2CB, TUBB4A, PPP2R1A, PSMA2, SMC3, UBE2S, |
| | PSMD3, KPNB1, PSMD11, ANAPC1, NUP98, PSMD9, NUP107, FBXO5, |
| | PPP2R5D, LMNB1, NUP155, PPP2CA, CENPA, PSMD14, CHMP3, SUMO1, |
| | CDC20, NSL1, CENPF, STAG1, NUP43, CENPL, TMPO, KIF18A, PDS5A, |
| | ZWINT, CENPK, TUBA1B, B9D2, NUP85, PSMF1, PSMB2, TUBA4A, SEM1, |
| | PSMA1, SGO1, CDC16, CHMP2A, CLIP1, PSME3, RAN, VPS4A, CCNB1, |
| | CDCA8, ESPL1, DYNC1LI, PSMB7, TUBB2A, TUBB2B, PPP2R1B, KNL1, |
| | CENPO, NUP54, CENPE, NUP58, KIF2B, PSMB6, ITGB3BP, KIF2C, PSMA5, |
| | NUF2, LBR, RPS27A, CENPC, CDCA5, HDAC8, CHMP7, INCENP, DSN1, |
| | UBC, CENPU, TUBA3E, SPC25, CENPH, RANBP2, AHCTF1, PPP2R5E, |
| | CC2D1B, BUB3, PSMA8, SKA1, NUP205, BUB1B, SEC13, CCNB2, |
| | DYNC1I1, PSMD4, PSMB4, TAOK1, PMF1, LMNA, PSMC2, SPC24, |
| | LEMD2, NUP35, SGO2, CLASP2, PSMD6, MAD2L1, PTTG1, CHMP4C, |
| | RAD21, PSMC3, CENPN, NDEL1, PLK1, TUBA1A, TUBA1C, MIS12, BUB1, |
| | UBE2E1, CDK1, UBB, TUBB8B, PSMD1, LEMD3, ZWILCH, UBE2C, |
| | PSMD2, CKAP5, CENPS, BANF1, TUBB6, CHMP6, ANAPC2, CDC26, |
| | ANKLE2, RPS27, TUBAL3, AURKB, RCC2, RCC1, IST1, SKA2, TUBA8, |
| | KNTC1, PSMD13, PPP1CC, ERCC6L, TUBB4B, CENPP, POM121, ANAPC7, |
| | DYNC1H, PSMD12, TUBA3C, PSMB8, PSMB10, NUP62, PPP2R2A, UBA52, |
| | PSMB11, PSMB9, TUBA4B, CHMP4A, TUBB3, TUBB8, DYNLL2, PSMB3 |
| REACTOME_{—} | PRKAR2B, PAFAH1B1, OFD1, SDCCAG8, CUL1, PPP1R12A, PRKACA, |
| REGULATION_OF_{—} | FBXW11, NDE1, CLASP1, PPP1R12B, DYNC1I2, PCM1, HSP90AA1, |
| PLK1_ACTIVITY_AT_{—} | AURKA, DYNLL1, HAUS4, NINL, MAPRE1, CEP76, CEP192, CCP110, |
| G2_M_TRANSITION | CEP152, TUBB4A, PPP2R1A, CEP41, YWHAE, CEP164, CEP72, SKP1, |
| | CEP70, ALMS1, NEK2, CNTRL, OPTN, CEP250, TUBA4A, AKAP9, AJUBA, |
| | HAUS8, TUBG1, CCNB1, BORA, ODF2, CDK5RAP2, DCTN3, HAUS2, |
| | ACTR1A, NEDD1, CSNK1D, CEP131, PLK4, RPS27A, CETN2, HAUS6, |
| | CEP78, UBC, CENPJ, HAUS1, CCNB2, PCNT, CEP57, BTRC, PLK1, RAB8A, |
| | TUBA1A, YWHAG, CDK1, UBB, CEP135, DCTN2, CKAP5, SSNA1, CEP63, |
| | TUBB, TUBB4B, DYNC1H1, SFI1, CEP290, DCTN1, CEP43, HAUS7, |
| | PPP1CB, CSNK1E, HAUS3, UBA52, HAUS5 |
| REACTOME_{—} | CFTR, MAD1L1, KDM1A, DVL2, PAFAH1B1, NOX1, BTK, WAS, NUP160, |
| RHO_GTPASE_{—} | CENPQ, CDH1, SPDL1, CTNNA1, GOPC, CYBA, CYFIP2, PPP1R12, |
| EFFECTORS | NCKAP1, PKN2, ROPN1, MYLK, PPP2R5A, RHOA, PRKCZ, ROCK1, |
| | KIF2A, PPP2R5B, NUP133, PFN2, CDC42, NDE1, CLASP1, NOX3, NUP37, |
| | ACTB, TUBA3D, PAK3, DYNC1I2, PIK3C3, PPP2R5C, NDC80, XPO1, |
| | SEH1L, CTTN, ZW10, DYNLL1, BIRC5, NUDC, MYL6, ABL1, MAPK1, |
| | CENPM, MYH9, NCF4, RANGAP1, DAAM1, TUBB1, MYL9, MAPRE1, |
| | CENPI, MAPK3, CENPT, PPP2CB, TUBB4A, LIN7B, KLC3, MYH14, |
| | PPP2R1A, H2AZ2, WASL, LIMK1, KDM4C, DVL1, PFN1, YWHAE, NUP98, |
| | ARPC3, CDKN1B, NUP107, MAPK14, WASF1, PPP2R5D, SRF, PPP2CA, |
| | RTKN, ACTR3, CENPA, WIPF1, NCF2, CDC20, NSL1, CENPF, KIF14, |
| | MYL12B, RHOQ, NUP43, CENPL, KIF18A |
| REACTOME_{—} | CFTR, MAD1L1, KDM1A, ARHGAP33, DVL2, ARHGAP44, PAFAH1B1, |
| SIGNALING_{—} | NOX1, RHOBTB2, BTK, WAS, RALBP1, NUP160, FAM13B, ARHGAP31, |
| BY_RHO_{—} | CENPQ, DEPDC1B, TRIO, CDH1, SPDL1, CTNNA1, ARAP2, ARHGAP6, |
| GTPASES | GOPC, ARHGEF5, CYBA, CYFIP2, GDI2, PPP1R12A, NCKAP1, PKN2, |
| | ROPN1, MYLK, PPP2R5A, NGEF, MYO9A, RHOA, PRKCZ, ROCK1, KIF2A, |
| | PPP2R5B, NUP133, PFN2, CDC42, ARHGAP10, RHOBTB1, NDE1, CLASP1, |
| | NOX3, ARHGEF10, NUP37, ACTB, ARHGAP15, TUBA3D, ARHGEF1, |
| | PPP1R12B, PAK3, DYNC1I2, PIK3C3, PPP2R5C, OPHN1, NDC80, XPO1, |
| | SEH1L, CTTN, ZW10, ARHGAP28, DYNLL1, GMIP, BIRC5, ARHGAP4, |
| | NUDC, PLEKHG2, MYL6, ABL1, SYDE2, MYO9B, MAPK1, CENPM, |
| | MYH9, NCF4, RANGAP1, SOS2, DAAM1, ARHGAP5, TUBB1, MYL9, |
| | MAPRE1, MCF2, FGD1, CENPI, ARHGEF7, MAPK3, CENPT, RHOV, |
| | PPP2CB, TUBB4A, LIN7B |

TABLE 8C
The 60 genes, from the 73 gene sets identified by set based analysis, that showed significant
differential expression in C4-2 cells after treatment with abiraterone, but not in LNCaP
cells treated with abiraterone, and in neither cell line treated with enzalutamide.

AGR2, KNL1, FMNL2, PRC1, HJURP, PSRC1, ASPM, OPA1, CCNB2, TAX1BP3, ESCO2,
PRKCD, CKAP2L, RACGAP1, PLK4, DLGAP5, TROAP, DVL3, KPNA2, CALM1,
SELENOS, UBE2C, THOC6, RRM1, STUB1, ARHGAP21, GIT1, PPP2R5C, CBX1, KIF14,
RB1, MTCL1, DYNC1LI1, PPP1R9B, TP53, SOS1, OAZ1, APP, OPTN, CLTC, GDAP1,
SMAD3, DNMT3A, WFS1, SETDB2, GDI1, AGO4, CUL7, ACTG1, FANCD2, ITGB1,
STMN1, ARHGAP12, XPO1, POC1B, SLC19A1, ERN1, SERP1, HMGB3, CALM3

### Example 4

### Tumor Transcriptomic Profiling Predicts the Patients' Clinical Response to Abi-Onv Combination Therapy

Materials and Methods

The abiraterone-onvansertib synergy gene signature (described in Example 3, see Tables 12A and 12B) was applied to a dataset containing gene expression data from 32,000 prostate cancer specimens provided by Decipher Biosciences Inc. The synergy score derived from the gene signature was calculated for each specimen's gene expression data. Synergy scores were compared amongst molecular subtypes as predefined by Decipher Biosciences.

Results

Based on a transcriptomic analysis of 32,000 prostate cancer specimens, Decipher Biosciences prostate subtyping classifier has characterized 4 molecular subtypes: Luminal A (LA), Luminal Proliferating (LP), Basal Immune (BI) and Basal (B).

The Abi-Onv synergy gene signature (Example 3, Table 7A) was applied to the 32,000 prostate cancer specimens by Decipher Biosciences.

FIG. 10 is a graph showing change in Abi-Onv synergy signature score in prostate cancer specimens characterized into four molecular subtypes: Luminal A (LA), Luminal Proliferating (LP), Basal Immune (BI) and Basal (B).

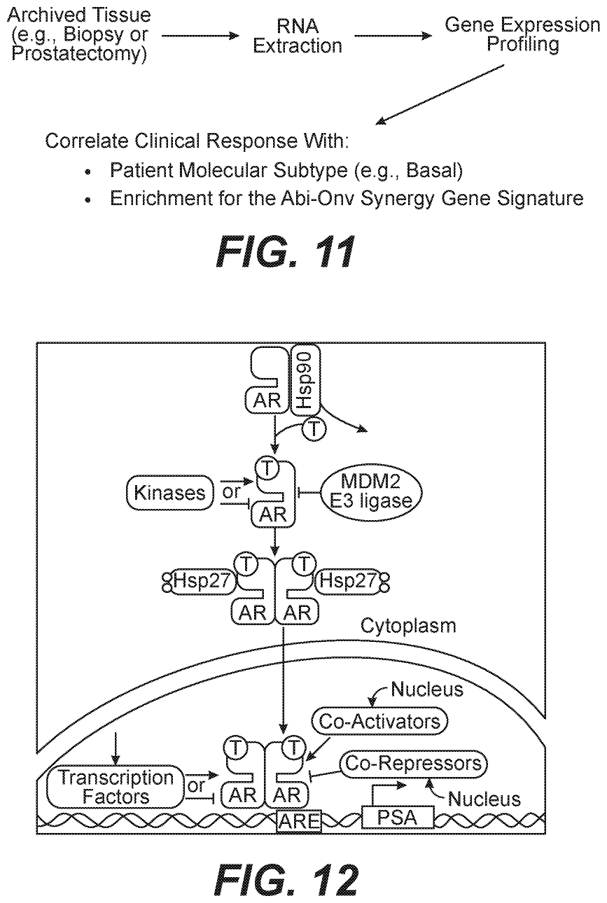

FIG. 11 is a flow chart showing steps in tumor transcriptomic profiling to predict clinical response.

The Abi-Onv synergy gene signature was significantly enriched in the Basal subtype compared to the other subtypes (p<0.001) (FIG. 10). The Basal subtype represents about ⅓ of mCRPC patients and is associated with lower responses to standard androgen deprivation therapy. Therefore, patients with basal subtype tumors may have a greater likelihood to respond to the disclosed combination therapies (e.g., Abi-Onv combination). Tumor transcriptomic profiling (e.g., by a method described in FIG. 11) could predict the patients' clinical response to Abi-Onv or other disclosed combination therapy (e.g., in patients with particular prostate cancer molecular subtypes).

Altogether, the data in the Examples show that abiraterone and PLK1i, including onvansertib, synergize in an AR-independent manner in vitro and in vivo. Abiraterone's AR-independent effects include induction of a mitosis related gene signature and disruption of mitotic spindle orientation, which is believed to render some cancer cells more sensitive to PLK1i. A phase 2 clinical trial is underway testing this combination in mCRPC. The correlative value of the synergy specific gene signature is also being tested using archival tissues.

Unless defined otherwise, all technical and scientific terms used herein have the same meanings as commonly understood by one of skill in the art to which the invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for selecting a treatment regimen or evaluating the likelihood of efficacious treatment or the status of treatment of a subject having cancer comprising:
(a) analyzing the expression of one or more genes or gene products involved in mitosis, meiosis, the mitotic spindle, mitotic spindle assembly and/or checkpoint, microtubule organization, the centromere, the kinetochore, G2M checkpoint, E2F target genes, or combinations thereof in a sample from the subject to determine if the patient is likely to benefit from treatment with a combination of an antiandrogen or androgen antagonist in combination with an effective amount of a Plk inhibitor;
(b) optionally selecting for treatment a subject whose sample is characterized by expression or up-regulation of the one or more genes or gene products analyzed in (a); and
(c) optionally administering to the selected subject an effective amount of an antiandrogen or androgen antagonist in combination with an effective amount of a Plk inhibitor.

2. The method of claim 1, wherein the subject was previously treated with the antiandrogen or androgen antagonist.

3. The method of claim 1, wherein the sample is selected from the group comprising cancer cells, blood sample, bone-marrow aspirate, ascites fluid, and tumor biopsy.

4. The method of claim 3, wherein the analysis is performed on isolated tumor cells, circulating tumor cells (CTCs), or cell-free RNA/exosomal RNA isolated from the blood sample.

5. The method of claim 1, wherein the analysis comprises quantification of RNA and/or protein expression.

6. The method of claim 5 further comprising quantification of genomic amplification or copy number variation, a cytological assay, or combinations thereof.

7. The method of claim 1, wherein the sample is treated with an antiandrogen or androgen antagonist before performing the analysis in (a).

8. The method of claim 7, wherein the sample exhibits expression or up-regulation of the one or more genes following treatment with the antiandrogen or androgen antagonist.

9. The method of claim 7, wherein the sample exhibits expression or up-regulation of the one or more genes relative to their expression in a sample not treated with the antiandrogen or androgen antagonist.

10. The method of claim 1, wherein the sample exhibits expression or up-regulation of the one or more genes relative to their expression in a sample from a subject who has not been administered the antiandrogen or androgen antagonist.

11. A method of treating cancer in a subject in need thereof comprising administering to the subject an effective amount of an antiandrogen or androgen antagonist in combination with an effective amount of a Plk inhibitor, wherein the cancer is characterized by expression or up-regulation of one or more genes or gene products involved in mitosis, meiosis, the mitotic spindle, mitotic spindle assembly and/or checkpoint, microtubule organization, the centromere, the kinetochore, G2M checkpoint, E2F target genes or combinations thereof; and monitoring expression to determine the likelihood the antiandrogen or androgen antagonist in combination with an effective amount of a Plk inhibitor is effective in reducing cancer size, proliferation, viability, or metastasis.

12. The method of claim 11, wherein the subject was previously treated with the antiandrogen or androgen antagonist.

13. The method of claim 11, wherein the expression or up-regulation of the one or more genes or gene products is relative to their expression in a cancer from a subject who has not been administered the antiandrogen or androgen antagonist.

14. The method of claim 1, wherein the one or more genes or gene products are selected from the genes listed Table 1, Table 2, Table 5A, Table 6A, Table 7A, Table 8B, or Table 8C.

15. The method of claim 14, wherein the one or more genes comprise one or more gene sets selected from Table 2.

16. The method of claim 1, wherein administration of the combination of the antiandrogen or androgen antagonist and the Plk inhibitor reduces cancer cell proliferation or cancer cell viability in the subject.

17. The method of claim 1, wherein administration of the combination of the antiandrogen or androgen antagonist and the Plk inhibitor reduces cancer cell proliferation or viability in the subject to a greater degree than administering to the subject the same amount of antiandrogen or androgen antagonist alone or the same amount of Plk inhibitor alone.

18. The method of claim 17, wherein the reduction in cancer cell proliferation or viability in the subject is more than the additive reduction achieved by administering the antiandrogen or androgen antagonist alone or the polo-like kinase inhibitor alone.

19. The method of claim 1, wherein the antiandrogen or androgen antagonist is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the Plk inhibitor.

20. The method of claim 1, wherein the Plk inhibitor is administered to the subject 1, 2, 3, 4, 5, 6, 8, 10, 12, 18, or 24 hours, 1, 2, 3, 4, 5, 6, or 7 days, 1, 2, 3, or 4 weeks, or any combination thereof prior to administration of the antiandrogen or androgen antagonist.

21. The method of claim 1, wherein the antiandrogen or androgen antagonist is administered daily and the Plk inhibitor is administered on days 1 through 5 of a 14-day or 21-day cycle.

22. The method of claim 1, wherein the antiandrogen or androgen antagonist is administered daily and the Plk inhibitor is administered on days 1 through 14 of a 21-day cycle.

23. The method of claim 1, wherein the antiandrogen or androgen antagonist is administered to the subject at the same time as the Plk inhibitor.

24. The method of claim 1, wherein the antiandrogen or androgen antagonist is selected from the group consisting of abiraterone, TOK-001, and mixtures thereof.

25. The method of claim 24, wherein the antiandrogen or androgen antagonist is abiraterone, or a prodrug, analog, or derivative, or pharmaceutically acceptable salt thereof.

26. The method of claim 25, wherein the dosage of abiraterone, or the prodrug, analog, derivative, or pharmaceutically acceptable salt thereof is between 250 mg-1,500 mg, inclusive.

27. The method of claim 1, wherein the class of Plk inhibitors is selected from the group consisting of dihydropteridinones, pyridopyrimidines, aminopyrimidines, substituted thiazolidinones, pteridine derivatives, dihydroimidazo[1,5-f]pteridines, metasubstituted thiazolidinones, benzyl styryl sulfone analogues, stilbene derivatives, 4,5-dihydro-1H-pyrazolo[4,3-h]quinazoline derivatives, and combinations thereof.

28. The method of claim 27, wherein the Plk inhibitor is selected from the group consisting of onvansertib, BI2536, volasertib (BI 6727), GSK461364, HMN-176, HMN-214, rigosertib (ON-01910), MLN0905, TKM-080301, TAK-960, or Ro3280.

29. The method of claim 28, wherein the Plk inhibitor is onvansertib.

30. The method of claim 29, wherein the dosage of onvansertib is between 6-60 mg/m², inclusive.

31. The method of claim 1, further comprising administering to the subject surgery, radiation therapy, and/or one or more additional active agents selected from the group consisting of a steroid, a chemotherapeutic agent that is not an antiandrogen or PLK inhibitor, a hematopoietic agent, an anti-infective agent, and combinations thereof.

32. The method of claim 31, wherein the steroid is prednisone.

33. The method of claim 31, wherein the chemotherapeutic agent is docetaxel.

34. The method of claim 1, wherein the cancer is selected from the group comprising prostate cancer, breast cancer, ovarian cancer, colorectal cancer, pancreatic cancer, head and neck cancer, bladder cancer, and acute myeloid leukemia.

35. The method of claim 34, wherein the prostate cancer is castrate resistant prostate cancer.

36. The method of claim 1, wherein the subject is a human.
